(19) United States
(12) Patent Application Publication
(10) Pub. No.: US 2010/0197908 A1
(43) Pub. Date: Aug. 5, 2010

(54) Pyridazine Derivatives with MCH Antagonistic Activity and Medicaments Comprising These Compounds

(75) Inventors: Thorsten Lehmann-Lintz, Ochsenhausen (DE); Dirk Stenkamp, Biberach (DE); Gerald Juergen Roth, Biberach (DE); Stephan Georg Mueller, Warthausen (DE); Joerg Kley, Mittelbiberach (DE); Armin Heckel, Biberach (DE); Klaus Rudolf, Warthausen (DE); Marcus Schindler, Oxford (GB); Ralf Lotz, Schemmerhofen (DE); Patrick Tielmann, Offenbach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 12/518,321

(22) PCT Filed: Dec. 10, 2007
(86) PCT No.: PCT/EP2007/063575

§ 371 Date: Apr. 1, 2010

(30) Foreign Application Priority Data
Dec. 11, 2006 (EP) 06125763.0

(51) Int. Cl. C07D 403/10; C07D 413/10; C07D 401/10; C07D 403/14; C07D 401/14

(52) U.S. Cl. 540/594; 544/114; 544/238

(57) ABSTRACT

The present invention relates to compounds of general formula I

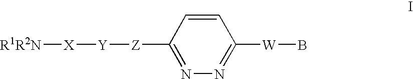

wherein the groups and radicals B, W, X, Y, Z, R¹, R², have the meanings given in claim 1. Moreover the invention relates to pharmaceutical compositions containing at least one compound according to the invention. By virtue of their MCH-receptor antagonistic activity the pharmaceutical compositions according to the invention are suitable for the treatment of metabolic disorders and/or eating disorders, particularly obesity, bulimia, anorexia, hyperphagia and diabetes.

The present invention relates to new pyridazine derivatives, the physiologically acceptable salts thereof as well as their use as MCH antagonists and their use in preparing a pharmaceutical preparation which is suitable for the prevention and/or treatment of symptoms and/or diseases caused by MCH or causally connected with MCH in some other way. The invention also relates to the use of a compound according to the invention for influencing eating behaviour and for reducing body weight and/or for preventing any increase in body weight in a mammal. It further relates to compositions and medicaments containing a compound according to the invention and processes for preparing them. Other aspects of this invention relate to processes for preparing the compounds according to the invention.

### BACKGROUND TO THE INVENTION

The intake of food and its conversion in the body is an essential part of life for all living creatures. Therefore, deviations in the intake and conversion of food generally lead to problems and also illness. The changes in the lifestyle and nutrition of humans, particularly in industrialised countries, have promoted morbid overweight (also known as corpulence or obesity) in recent decades. In affected people, obesity leads directly to restricted mobility and a reduction in the quality of life. There is the additional factor that obesity often leads to other diseases such as, for example, diabetes, dyslipidaemia, high blood pressure, arteriosclerosis and coronary heart disease. Moreover, high body weight alone puts an increased strain on the support and mobility apparatus, which can lead to chronic pain and diseases such as arthritis or osteoarthritis. Thus, obesity is a serious health problem for society.

The term obesity means an excess of adipose tissue in the body. In this connection, obesity is fundamentally to be seen as the increased level of fatness which leads to a health risk. There is no sharp distinction between normal individuals and those suffering from obesity, but the health risk accompanying obesity is presumed to rise continuously as the level of fatness increases. For simplicity's sake, in the present invention, individuals with a Body Mass Index (BMI), which is defined as the body weight measured in kilograms divided by the height (in metres) squared, above a value of 25 and more particularly above 30, are preferably regarded as suffering from obesity.

Apart from physical activity and a change in nutrition, there is currently no convincing treatment option for effectively reducing body weight. However, as obesity is a major risk factor in the development of serious and even life-threatening diseases, it is all the more important to have access to pharmaceutical active substances for the prevention and/or treatment of obesity. One approach which has been proposed very recently is the therapeutic use of MCH antagonists (cf. inter alia WO 01/21577, WO 01/82925).

Melanin-concentrating hormone (MCH) is a cyclic neuropeptide consisting of 19 amino acids. It is synthesised predominantly in the hypothalamus in mammals and from there travels to other parts of the brain by the projections of hypothalamic neurones. Its biological activity is mediated in humans through two different G-protein-coupled receptors (GPCRs) from the family of rhodopsin-related GPCRs, namely the MCH receptors 1 and 2 (MCH-1R, MCH-2R).

Investigations into the function of MCH in animal models have provided good indications for a role of the peptide in regulating the energy balance, i.e. changing metabolic activity and food intake [1,2]. For example, after intraventricular administration of MCH in rats, food intake was increased compared with control animals. Additionally, transgenic rats which produce more MCH than control animals, when given a high-fat diet, responded by gaining significantly more weight than animals without an experimentally altered MCH level. It was also found that there is a positive correlation between phases of increased desire for food and the quantity of MCH mRNA in the hypothalamus of rats. However, experiments with MCH knock-out mice are particularly important in showing the function of MCH. Loss of the neuropeptide results in lean animals with a reduced fat mass, which take in significantly less food than control animals.

The anorectic effects of MCH are presumably mediated in rodents through the G_{∀s}-coupled MCH-1R [3-6], as, unlike primates, ferrets and dogs, no second MCH receptor subtype has hitherto been found in rodents. After losing the MCH-1R, knock-out mice have a lower fat mass, an increased energy conversion and, when fed on a high fat diet, do not put on weight, compared with control animals. Another indication of the importance of the MCH system in regulating the energy balance results from experiments with a receptor antagonist (SNAP-7941) [3]. In long term trials the animals treated with the antagonist lose significant amounts of weight.

In addition to its anorectic effect, the MCH-1R antagonist SNAP-7941 also achieves additional anxiolytic and antidepressant effects in behavioural experiments on rats [3]. Thus, there are clear indications that the MCH-MCH-1R system is involved not only in regulating the energy balance but also in affectivity.

### LITERATURE

1. Qu, D., et al., A role for melanin-concentrating hormone in the central regulation of feeding behaviour. Nature, 1996. 380(6571): p. 243-7.
2. Shimada, M., et al., Mice lacking melanin-concentrating hormone are hypophagic and lean. Nature, 1998. 396(6712): p. 670-4.
3. Borowsky, B., et al., Antidepressant, anxiolytic and anorectic effects of a melanin-concentrating hormone-1 receptor antagonist. Nat Med, 2002. 8(8): p. 825-30.
4. Chen, Y., et al., Targeted disruption of the melanin-concentrating hormone receptor-1 results in hyperphagia and resistance to diet-induced obesity. Endocrinology, 2002. 143(7): p. 2469-77.
5. Marsh, D. J., et al., Melanin-concentrating hormone 1 receptor-deficient mice are lean, hyperactive, and hyperphagic and have altered metabolism. Proc Natl Acad Sci USA, 2002. 99(5): p. 3240-5.
6. Takekawa, S., et al., T-226296: A novel, orally active and selective melanin-concentrating hormone receptor antagonist. Eur J Pharmacol, 2002. 438(3): p. 129-35.

In the patent literature (WO 01/21577, WO 01/82925) amine compounds of the general formula

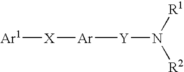

are proposed as MCH antagonists for the treatment of obesity.

Further patent publication related to amine compounds with MCH antagonistic activity are for example: WO 04/024702, WO 04/039780, WO 04/039764, WO 05/063239, WO 05/085221, WO 05/103031, WO 05/103032, WO 05/103029, WO 05/100285, WO 05/103002.

In the WO 03/068230, WO 2005/018557 (Pharmacia Corp.) substituted pyridinones are described. The WO 2004/087677 (Pharmacia Corp.) is related to pyrimidone derivatives and the WO 03/059891 as well as the WO 2005/007632 (Pharmacia Corp.) refer to pyridazinone derivatives. These compounds are described as modulators of p38 MAP kinase.

### AIM OF THE INVENTION

The aim of the present invention is to identify compounds which are especially effective as MCH antagonists. The invention also sets out to provide compounds which can be used to influence the eating habits of mammals and achieve a reduction in body weight, particularly in mammals, and/or prevent an increase in body weight.

The present invention further sets out to provide new pharmaceutical compositions which are suitable for the prevention and/or treatment of symptoms and/or diseases caused by MCH or otherwise causally connected to MCH. In particular, the aim of this invention is to provide pharmaceutical compositions for the treatment of metabolic disorders such as obesity and/or diabetes as well as diseases and/or disorders which are associated with obesity and diabetes. Other objectives of the present invention are concerned with demonstrating advantageous uses of the compounds according to the invention. The invention also sets out to provide a process for preparing the compounds according to the invention. Other aims of the present invention will be immediately apparent to the skilled man from the foregoing remarks and those that follow.

### OBJECT OF THE INVENTION

In a first aspect the present invention relates to pyridazine compounds of general formula I

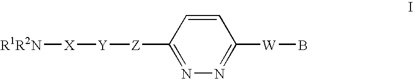

wherein
R¹, R² independently of one another denote H, C₁₋₈-alkyl or C₃₋₇-cycloalkyl, while the alkyl or cycloalkyl group may be mono- or polysubstituted by identical or different groups R¹¹, and a —CH₂— group in position 3 or 4 of a 5-, 6- or 7-membered cycloalkyl group may be replaced by —O—, —S— or —NR¹³—; or
   R² denotes a C₁₋₃-alkylen bridge which is linked to the group Y, wherein the alkylene bridge may be substituted with one or more C₁₋₃-alkyl-groups, and R¹ is defined as hereinbefore or denotes a group selected from C₁₋₄-alkyl-CO—, C₁₋₄-alkyl-O—CO—, (C₁₋₄-alkyl)NH—CO— or (C₁₋₄-alkyl)₂N—CO— wherein alkyl-groups may be mono- or polyfluorinated; or
   R¹ and R² form a C₃₋₈-alkylene bridge, wherein a —CH₂— group not adjacent to the N atom of the R¹R²N— group may be replaced by —CH═N—, —CH═CH—, —O—, —S—, —SO—, —(SO₂)—, —CO—, —O(═CH₂)—, —O(═N—O—(C₁₋₄-alkyl))— or —NR¹³—,
   while in the case when R¹ and R² form an alkylene bridge in the alkylene bridge one or more H atoms may be replaced by identical or different groups R¹⁴, and
   the alkylene bridge defined hereinbefore may be substituted by one or two identical or different carbo- or heterocyclic groups Cy in such a way that the bond between the alkylene bridge and the group Cy is made
      via a single or double bond,
      via a common C atom forming a spirocyclic ring system,
      via two common adjacent C and/or N atoms forming a fused bicyclic ring system or
      via three or more C and/or N atoms forming a bridged ring system;
X denotes a bridging group selected from the group consisting of —CH₂—, —CH₂—CH₂—, —CH₂—CH₂—CH₂—, —CH₂—CH₂—O— and —CH₂—CH₂—NR^{N}—, all of which may comprise one, two or three identical or different C₁₋₄-alkyl substituents, while two alkyl groups may be joined together forming a 3 to 7-membered cyclic group, and while in a C₂₋₃-alkylene bridge one or two C atoms may be monosubstituted by R¹⁰; and
R¹⁰ is selected from the group consisting of hydroxy, hydroxy-C₁₋₃-alkyl, C₁₋₄-alkoxy or C₁₋₄-alkoxy-C₁₋₃-alkyl; and
Y denotes a 5- to 6-membered aromatic carbocyclic group, which may contain 1, 2 or 3 heteroatoms independently selected from N, O and/or S; which cyclic group may be mono- or polysubstituted by identical or different substituents R²⁰;
Z denotes —CH₂—CH₂—, —C(═O)—CH₂—, —O(═CH₂)—CH₂—, —C(OH)H—CH₂— or —CH₂—C(OH)H—, all of which may be mono- or polysubstituted with substituents independently from each other selected from C₁₋₃-alkyl;
W is selected from the group consisting of —CH₂—CH₂—, —CH₂—O—, —O—CH₂—, —CH═CH—, —CH₂—NR^{N}—, —NR^{N}—CH₂—, —CH₂—, —O—, —S— and —NR^{N}—, wherein one or more H-atoms may be replaced independently of each other by C₁₋₃-alkyl;
R^{N} independently of one another denote H, C₁₋₄-alkyl, formyl, C₁₋₃-alkylcarbonyl or C₁₋₃-alkylsulfonyl; and
B is a 5- or 6-membered unsaturated or aromatic carbocyclic group which may contain 1, 2, 3 or 4 heteroatoms independently selected from N, O and/or S; which cyclic group may be mono- or polysubstituted by identical or different substituents R²⁰; or
   denotes C₁₋₆-alkyl, C₃₋₇-cycloalkyl or C₃₋₇-cycloalkyl-C₁₋₃-alkyl, wherein said alkyl-, cycloalkyl- or cycloalkylalkyl-groups may be mono- or poly-substituted independently of each other by R¹⁴; and where in cycloalkyl-groups one or two —CH₂-groups may be replaced independently of each other by —O—, —S—, —NR¹³— or —C(═O)—; and
Cy denotes a carbo- or heterocyclic group selected from one of the following meanings
   a saturated 3- to 7-membered carbocyclic group,
   an unsaturated 4- to 7-membered carbocyclic group,
   a phenyl group,
   a saturated 4- to 7-membered or unsaturated 5- to 7-membered heterocyclic group with an N, O or S atom as heteroatom,
   a saturated or unsaturated 5- to 7-membered heterocyclic group with two or more N atoms or with one or two N atoms and an O or S atom as heteroatoms,
   an aromatic heterocyclic 5- or 6-membered group with one or more identical or different heteroatoms selected from N, O and/or S,
   while the above-mentioned saturated 6- or 7-membered groups may also be present as bridged ring systems with an imino, (C₁₋₄-alkyl)-imino, methylene, ethylene, (C₁₋₄-alkyl)-methylene or di-(C₁₋₄-alkyl)-methylene bridge, and
   while the above-mentioned cyclic groups may be mono- or polysubstituted at one or more C atoms by identical or different groups R²⁰, or in the case of a phenyl group may also additionally be monosubstituted by nitro, and/or one or more NH groups may be substituted by R²¹; and
   while in the above-mentioned saturated or unsaturated carbo- or heterocyclic groups a —CH₂-group may be replaced by a —C(═O)— group;
R¹¹ denotes halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N—, R¹⁸R¹⁹N—CO— or Cy, while in the above-mentioned groups one or more C atoms may be substituted independently of one another by substituents selected from halogen, OH, CN, CF₃, C₁₋₃-alkyl, C₁₋₃-alkoxy, hydroxy-C₁₋₃-alkyl;
R¹³ has one of the meanings given for R¹⁷ or denotes formyl;
R¹⁴ denotes halogen, cyano, C₁₋₆-alkyl, C₂₋₆-alkenyl, C₂₋₆-alkynyl, R¹⁵—O—, R¹⁵—O—CO—, R¹⁵—CO—, R¹⁵—CO—O—, R¹⁶R¹⁷N—, HCO—NR¹⁵—, R¹⁸R¹⁹N—CO—, R¹⁸R¹⁹N—CO—NH—, R¹⁵—O—C₁₋₃-alkyl-, R¹⁵—O—CO—C₁₋₃-alkyl-, R¹⁵—SO₂—NH—, R¹⁵—SO₂—N(C₁₋₃-alkyl)-, R¹⁵—O—CO—NH—C₁₋₃-alkyl-, R¹⁵—SO₂—NH—C₁₋₃-alkyl-, R¹⁵—CO—C₁₋₃-alkyl-, R¹⁵—CO—O—C₁₋₃-alkyl-, R¹⁶R¹⁷N—C₁₋₃-alkyl-, R¹⁸R¹⁹N—CO—C₁₋₃-alkyl- or Cy-C₁₋₃-alkyl-,
R¹⁵ denotes H, C₁₋₄-alkyl, C₃₋₇-cycloalkyl, C₃₋₇-cycloalkyl-C₁₋₃-alkyl, phenyl, phenyl-C₁₋₃-alkyl, pyridinyl or pyridinyl-C₁₋₃-alkyl,
R¹⁶ denotes H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl or cyclo-C₃₋₆-alkyleneimino-C₂₋₆-alkyl,
R¹⁷ has one of the meanings given for R¹⁶ or denotes phenyl, phenyl-C₁₋₃-alkyl, pyridinyl, C₁₋₄-alkylcarbonyl, C₃₋₇-cycloalkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N—(C₁₋₄-alkylcarbonyl)-N—(C₁₋₄-alkyl)-amino-C₂₋₃-alkyl, C₁₋₄-alkylamino-carbonyl, C₁₋₄-alkylsulphonyl, C₁₋₄-alkylsulphonylamino-C₂₋₃-alkyl or N—(C₁₋₄-alkylsulphonyl)-N(—C₁₋₄-alkyl)-amino-C₂₋₃-alkyl;
R¹⁸, R¹⁹ independently of one another denote H or C₁₋₆-alkyl wherein R¹⁸, R¹⁹ may be linked to form a C₃₋₆-alkylene bridge, wherein a —CH₂— group not adjacent to an N atom may be replaced by —O—, —S-—, —SO—, —(SO₂)—, —CO—, —C(═CH₂)— or —NR¹³—;
R²⁰ denotes halogen, hydroxy, cyano, nitro, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²—C₁₋₃-alkyl or has one of the meanings given for R²²; and
R²¹ denotes C₁₋₄-alkyl, ω-hydroxy-C₂₋₆-alkyl, ω-C₁₋₄-alkoxy-C₂₋₆-alkyl, ω-C₁₋₄-alkylamino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulphonyl, aminosulphonyl, C₁₋₄-alkylaminosulphonyl, di-C₁₋₄-alkylaminosulphonyl or cyclo-C₃₋₆-alkylene-imino-sulphonyl,
R²² denotes pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylamino-carbonyl, di-(C₁₋₄-alkyl)-aminocarbonyl, cyclo-C₃₋₆-alkyl-amino-carbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulphonyl, C₁₋₄-alkyl-sulphinyl, C₁₋₄-alkyl-sulphonylamino, C₁₋₄-alkyl-sulphonyl-N—(C₁₋₄-alkyl)amino, amino, C₁₋₄-alkylamino, di-(C₁₋₄-alkyl)-amino, C₁₋₄-alkyl-carbonyl-amino, C₁₋₄-alkyl-carbonyl-N—(C₁₋₄-alkyl)amino, cyclo-C₃₋₆-alkyleneimino, phenyl-C₁₋₃-alkylamino, N—(C₁₋₄-alkyl)-phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazin-yl)carbonyl, aminocarbonylamino or C₁₋₄-alkylaminocarbonylamino,
   while in the above-mentioned groups and radicals, particularly in W, X, Z, R^{N}, R¹⁰, R¹¹, R¹³ to R²², in each case one or more C atoms may additionally be mono- or polysubstituted by F and/or in each case one or two C atoms independently of one another may additionally be monosubstituted by Cl or Br and/or in each case one or more phenyl rings may additionally comprise independently of one another one, two or three substituents selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl- and di-(C₁₋₃-alkyl)-amino-C₁₋₃-alkyl and/or may be monosubstituted by nitro, and
   the H atom of any carboxy group present or an H atom bound to an N atom may in each case be replaced by a group which can be cleaved in vivo,
      the tautomers, the diastereomers, the enantiomers, the mixtures thereof and the salts, in particular pharmaceutically acceptable salts, thereof.

The invention also relates to the compounds in the form of the individual optical isomers, mixtures of the individual enantiomers or racemates, in the form of the tautomers and in the form of the free bases or corresponding acid addition salts with pharmacologically acceptable acids. The subject of the invention also includes the compounds according to the invention, including their salts, wherein one or more hydrogen atoms are replaced by deuterium.

This invention also includes the physiologically acceptable salts of the compounds according to the invention as described above and hereinafter.

Also covered by this invention are compositions containing at least one compound according to the invention and/or a salt according to the invention optionally together with one or more physiologically acceptable excipients.

Also covered by this invention are pharmaceutical compositions containing at least one compound according to the invention and/or a salt according to the invention optionally together with one or more inert carriers and/or diluents.

This invention also relates to the use of at least one compound according to the invention and/or a salt according to the invention or one of the physiologically acceptable salts thereof, for influencing the eating behaviour of a mammal.

The invention further relates to the use of at least one compound according to the invention and/or a salt according to the invention or one of the physiologically acceptable salts thereof, for reducing the body weight and/or for preventing an increase in the body weight of a mammal.

The invention also relates to the use of at least one compound according to the invention and/or a salt according to the invention or one of the physiologically acceptable salts thereof, for preparing a pharmaceutical composition with an MCH receptor-antagonistic activity, particularly with an MCH-1 receptor-antagonistic activity.

This invention also relates to the use of at least one compound according to the invention and/or a salt according to the invention or one of the physiologically acceptable salts thereof, for preparing a pharmaceutical composition which is suitable for the prevention and/or treatment of symptoms and/or diseases which are caused by MCH or are otherwise causally connected with MCH.

A further object of this invention is the use of at least one compound according to the invention and/or a salt according to the invention or one of the physiologically acceptable salts thereof, for preparing a pharmaceutical composition which is suitable for the prevention and/or treatment of metabolic disorders and/or eating disorders, particularly obesity, bulimia, bulimia nervosa, cachexia, anorexia, anorexia nervosa and hyperphagia.

The invention also relates to the use of at least one compound according to the invention and/or a salt according to the invention or one of the physiologically acceptable salts thereof, for preparing a pharmaceutical composition which is suitable for the prevention and/or treatment of diseases and/or disorders associated with obesity, particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis and gonitis.

In addition the present invention relates to the use of at least one compound according to the invention and/or a salt according to the invention or one of the physiologically acceptable salts thereof, for preparing a pharmaceutical composition which is suitable for the prevention and/or treatment of hyperlipidaemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia and hormonal disorders.

The invention also relates to the use of at least one compound according to the invention and/or a salt according to the invention or one of the physiologically acceptable salts thereof, for preparing a pharmaceutical composition which is suitable for the prevention and/or treatment of urinary problems, such as for example urinary incontinence, overactive bladder, urgency, nycturia and enuresis.

The invention further relates to the use of at least one compound according to the invention and/or a salt according to the invention or one of the physiologically acceptable salts thereof, for preparing a pharmaceutical composition which is suitable for the prevention and/or treatment of dependencies and/or withdrawal symptoms.

The invention further relates to processes for preparing for preparing a pharmaceutical composition according to the invention, characterised in that at least one compound according to the invention and/or a salt according to the invention is incorporated in one or more inert carriers and/or diluents by a non-chemical method.

The invention also relates to a pharmaceutical composition containing a first active substance which is selected from the compounds according to the invention and/or the corresponding salts or one of the physiologically acceptable salts thereof, as well as a second active substance which is selected from the group consisting of active substances for the treatment of diabetes, active substances for the treatment of diabetic complications, active substances for the treatment of obesity, preferably other than MCH antagonists, active substances for the treatment of high blood pressure, active substances for the treatment of dyslipidaemia or hyperlipidaemia, including arteriosclerosis, active substances for the treatment of arthritis, active substances for the treatment of anxiety states and active substances for the treatment of depression, optionally together with one or more inert carriers and/or diluents.

Moreover, in one aspect, the invention relates to processes for preparing compounds of formula as described hereinafter.

The starting materials and intermediate products used in the synthesis according to the invention are also a subject of this invention.

### DETAILED DESCRIPTION OF THE INVENTION

Unless otherwise specified, the groups, residues and substituents, particularly B, W, X, Y, Z, Cy, R¹, R², R¹⁰, R¹¹, R¹³ to R²², R^{N}, have the meanings given hereinbefore.

If groups, residues and/or substituents occur more than once in a compound, they may have the same or different meanings in each case.

If R¹ and R² are not joined together via an alkylene bridge, R¹ and R² independently of one another preferably denote a C₁₋₈-alkyl or C₃₋₇-cycloalkyl group which may be mono- or polysubstituted by identical or different groups R¹¹, while a —CH₂— group in position 3 or 4 of a 5-, 6- or 7-membered cycloalkyl group may be replaced by —O—, —S— or —NR¹³—, while one or both of the groups R¹ and R² may also represent H.

Preferred meanings of the group R¹¹ are F, C₁₋₆-alkyl, C₂₋₆-alkenyl, C₂₋₆-alkynyl, R¹⁵—O—, cyano, R¹⁶R¹⁷N, C₃₋₇-cycloalkyl, cyclo-C₃₋₆-alkyleneimino, pyrrolidinyl, N—(C₁₋₄-alkyl)-pyrrolidinyl, piperidinyl, N—(C₁₋₄-alkyl)-piperidinyl, phenyl, pyridyl, pyrazolyl, thiazolyl, imidazolyl, while in the above-mentioned groups and radicals one or more C atoms may be mono- or polysubstituted independently of one another by F, C₁₋₃-alkyl, C₁₋₃-alkoxy or hydroxy-C₁₋₃-alkyl, and/or one or two C atoms may be monosubstituted independently of one another by Cl, Br, OH, CF₃ or CN, and the above-mentioned cyclic groups may be mono- or polysubstituted at one or more C atoms by identical or different radicals R²⁰, or in the case of a phenyl group may also additionally be monosubstituted by nitro, and/or one or more NH groups may be substituted by R²¹. If R¹¹ has one of the meanings R¹⁵—O—, cyano, R¹⁶R¹⁷N or cyclo-C₃₋₆-alkyleneimino, the C atom of the alkyl or cycloalkyl group substituted by R¹¹ is preferably not directly connected to a heteroatom, such as for example to the group —N—X—.

Preferably the groups R¹, R² independently of one another represent H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N—(C₁₋₄-alkyl)-pyrrolidin-3-yl, pyrrolidinyl-C₁₋₃-alkyl, N—(C₁₋₄-alkyl)-pyrrolidinyl-C₁₋₃-alkyl, piperidin-3-yl, piperidin-4-yl, N—(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-yl, tetrahydropyran-4-yl, tetrahydrofuran-2-ylmethyl, tetrahydrofuran-3-ylmethyl, phenyl-C₁₋₃-alkyl, pyridyl-C₁₋₃-alkyl, pyrazolyl-C₁₋₃-alkyl, thiazolyl-C₁₋₃-alkyl or imidazolyl-C₁₋₃-alkyl, while in the above-mentioned groups and radicals one or more C atoms independently of one another may be mono- or polysubstituted by F, C₁₋₃-alkyl or hydroxy-C₁₋₃-alkyl, and/or one or two C atoms independently of one another may be monosubstituted by Cl, Br, OH, CF₃ or CN, and the above-mentioned cyclic groups may be mono- or polysubstituted at one or more C atoms by identical or different radicals R²⁰, in the case of a phenyl group may also additionally be monosubstituted by nitro, and/or one or more NH groups may be substituted by R²¹. Preferred substituents of the above-mentioned phenyl, pyridyl, pyrazolyl, thiazolyl or imidazolyl groups are selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl and di-(C₁₋₃-alkyl)-amino-C₁₋₃-alkyl, while a phenyl group may also be monosubstituted by nitro.

Particularly preferred definitions of the groups R¹ and/or R² are selected from the group consisting of H, C₁₋₄-alkyl, hydroxy-C₁₋₄-alkyl, C₃₋₅-alkenyl, C₃₋₅-alkynyl, C₃₋₇-cycloalkyl, hydroxy-C₃₋₇-cycloalkyl, dihydroxy-C₃₋₆-alkyl, C₃₋₇-cycloalkyl-C₁₋₃-alkyl, tetrahydropyran-3-yl, tetrahydropyran-4-yl, tetrahydrofuran-2-ylmethyl, tetrahydrofuran-3-ylmethyl, (hydroxy-C₃₋₇-cycloalkyl)-C₁₋₃-alkyl, C₁₋₄-alkoxy-C₂₋₃-alkyl, hydroxy-C₁₋₄-alkoxy-C₂₋₃-alkyl, C₁₋₄-alkoxy-C₁₋₄-alkoxy-C₂₋₃-alkyl, di-(C₁₋₃-alkyl)amino-C₂₋₃-alkyl, pyrrolidin-N-yl-C₂₋₃-alkyl, piperidin-N-yl-C₂₋₃-alkyl, pyridylmethyl, pyrazolylmethyl, thiazolylmethyl and imidazolylmethyl, while an alkyl, cycloalkyl or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl or CN. The above-mentioned phenyl, pyridyl, pyrazolyl, thiazolyl or imidazolyl group may be mono- or polysubstituted with a substituent independently of each other selected from F, Cl, Br, I, cyano, C₁₋₃-alkyl, C₁₋₃-alkoxy, trifluoromethyl, hydroxy, amino, acetylamino, aminocarbonyl, while a phenyl group may also be monosubstituted by nitro.

Most particularly preferred groups R¹ and/or R² are selected from the group consisting of H, methyl, ethyl, n-propyl, i-propyl, 2-methylpropyl, 2-methoxyethyl, cyclopropyl, cyclopentyl, cyclohexyl, cyclopropylmethyl, cyclopentylmethyl, hydroxy-C₃₋₇-cycloalkyl, (hydroxy-C₁₋₃-alkyl)-hydroxy-C₃₋₇-cycloalkyl, dihydroxy-C₃₋₅-alkyl, 2-hydroxy-1-(hydroxymethyl)-ethyl, 1,1-di(hydroxymethyl)-ethyl, (1-hydroxy-C₃₋₆-cycloalkyl)-methyl, tetrahydropyran-3-yl, tetrahydropyran-4-yl, tetrahydrofuran-2-ylmethyl, tetrahydrofuran-3-ylmethyl, 2-hydroxyethyl, 3-hydroxypropyl, 2-hydroxy-2-methyl-propyl, di-(C₁₋₃-alkyl)aminoethyl, pyrrolidin-N-yl-ethyl, piperidin-N-ylethyl, pyridylmethyl, pyrazolylmethyl, thiazolylmethyl and imidazolylmethyl, while the above-mentioned groups may be mono- or polysubstituted by F and/or C₁₋₃-alkyl.

Examples of most particularly preferred groups R¹ and/or R² are therefore H, methyl, ethyl, n-propyl, i-propyl, 2-methylpropyl, 2-methoxyethyl, cyclopropyl, cyclopentyl, cyclohexyl, cyclopropylmethyl, cyclopentylmethyl, hydroxy-cyclopentyl, hydroxy-cyclohexyl, (hydroxymethyl)-hydroxy-cyclopentyl, (hydroxymethyl)-hydroxy-cyclohexyl, 2,3-dihydroxypropyl, (1-hydroxy-cyclopropyl)-methyl, tetrahydropyran-3-yl, tetrahydropyran-4-yl, tetrahydrofuran-2-ylmethyl, tetrahydrofuran-3-ylmethyl, 2-hydroxyethyl, 3-hydroxypropyl, 2-hydroxy-2-methyl-propyl, dimethylaminoethyl, pyridylmethyl, pyrazolylmethyl, (1-methyl-1H-pyrazol-4-yl)methyl, thiazolylmethyl, imidazolylmethyl, (1-methyl-1H-imidazol-2-yl)methyl. The beforementioned groups may be mono- or polysubstituted by F and/or C₁₋₃-alkyl.

Preferably, one of the groups R¹, R² has a meaning other than H; in particular both groups R¹, R² have a meaning other than H.

In case the group R² denotes a C₁₋₃-alkylen bridge which is linked to the group Y, preferably the definition of R¹ is in accordance with a preferred definition as described hereinbefore or R¹ denotes a group selected from C₁₋₄-alkyl-CO—, C₁₋₄-alkyl-O—CO—, (C₁₋₄-alkyl)NH—CO— or (C₁₋₄-alkyl)₂N—CO— wherein alkyl-groups may be mono- or polyfluorinated. In case R² is linked to the group Y, then R² preferably denotes —CH₂— or —CH₂—CH₂—, wherein the alkylene bridge may be substituted with one or more C₁₋₃-alkyl-groups. In case R² is linked to the group Y, then R¹ preferably denotes H or C₁₋₃-alkyl which may be mono- or polyfluorinated.

In case the groups R¹ and R² form an alkylene bridge, this is preferably a C₃₋₇-alkylene bridge or a C₃₋₇-alkylene bridge, wherein a —CH₂— group not adjacent to the N atom of the R¹R²N group is replaced by —CH═N—, —CH═CH—, —O—, —S—, —(SO₂)—, —CO—, —O(═N—O—(C₁₋₄-alkyl))— or —NR¹³—,

while in the alkylene bridge defined hereinbefore one or more H atoms may be replaced by identical or different groups R¹⁴, and
the alkylene bridge defined hereinbefore may be substituted with a carbo- or heterocyclic group cy in such a way that the bond between the alkylene bridge and the group Cy is made
via a single or double bond,
via a common C atom forming a spirocyclic ring system,
via two common adjacent C— and/or N atoms forming a fused bicyclic ring system or
via three or more C— and/or N atoms forming a bridged ring system.

Preferably also, R¹ and R² form an alkylene bridge such that R¹R²N— denotes a group which is selected from azetidine, pyrrolidine, piperidine, azepan, 2,5-dihydro-1H-pyrrole, 1,2,3,6-tetrahydro-pyridine, 2,3,4,7-tetrahydro-1H-azepine, 2,3,6,7-tetrahydro-1H-azepine, piperazine in which the free imine function is substituted by R¹³, piperidin-4-one, morpholine, thiomorpholine, 1-oxo-thiomorpholin-4-yl, 1,1-dioxo-thiomorpholin-4-yl, 4-C₁₋₄-alkoxy-imino-piperidin-1-yl and 4-hydroxyimino-piperidin-1-yl; or

a group which is particularly preferably selected from azetidine, pyrrolidine, piperidine, piperazine in which the free imine function is substituted by R¹³, and morpholine,
while according to the general definition of R¹ and R² one or more H atoms may be replaced by identical or different groups R¹⁴, and/or the above-mentioned groups may be substituted by one or two identical or different carbo- or heterocyclic groups Cy in a manner specified according to the general definition of R¹ and R², while the group Cy may be mono- or polysubstituted by R²⁰.

Particularly preferred groups Cy are C₃₋₇-cycloalkyl, aza-C₄₋₇-cycloalkyl, particularly cyclo-C₃₋₆-alkyleneimino, as well as 1-C₁₋₄-alkyl-aza-C₄₋₇-cycloalkyl, while the group Cy may be mono- or polysubstituted by R²⁰.

The C₃₋₈-alkylene bridge formed by R¹ and R², wherein —CH₂— groups may be replaced as specified, may be substituted, as described, by one or two identical or different carbo- or heterocyclic groups Cy, which may be substituted as specified hereinbefore.

In the event that the alkylene bridge is linked to a group Cy through a single bond, Cy is preferably selected from the group consisting of C₃₋₇-cycloalkyl, cyclo-C₃₋₆-alkyleneimino, imidazol, triazol, thienyl and phenyl.

In the event that the alkylene bridge is linked to a group Cy via a common C atom forming a spirocyclic ring system, Cy is preferably selected from the group consisting of C₃₋₇-cycloalkyl, aza-C₄₋₈-cycloalkyl, oxa-C₄₋₈-cycloalkyl, 2,3-dihydro-1H-quinazolin-4-one.

In the event that the alkylene bridge is linked to a group Cy via two common adjacent C and/or N atoms forming a fused bicyclic ring system, Cy is preferably selected from the group consisting of C₄₋₇-cycloalkyl, phenyl, thienyl.

In the event that the alkylene bridge is linked to a group Cy via three or more C and/or N atoms forming a bridged ring system, Cy preferably denotes C₄₋₈-cycloalkyl or aza-C₄₋₈-cycloalkyl.

In the event that the heterocyclic group R¹R²N— is substituted by a group Cy, the group Cy is preferably linked to the group R¹R²N— through a single bond, while Cy is preferably selected from the group consisting of C₃₋₇-cycloalkyl, cyclo-C₃₋₆-alkyleneimino, imidazol and triazol, while these groups may be substituted as specified, preferably by fluorine, C₁₋₃-alkyl, hydroxy-C₁₋₃-alkyl and hydroxy.

Particularly preferably the group

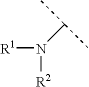

is defined according to one of the following partial formulae

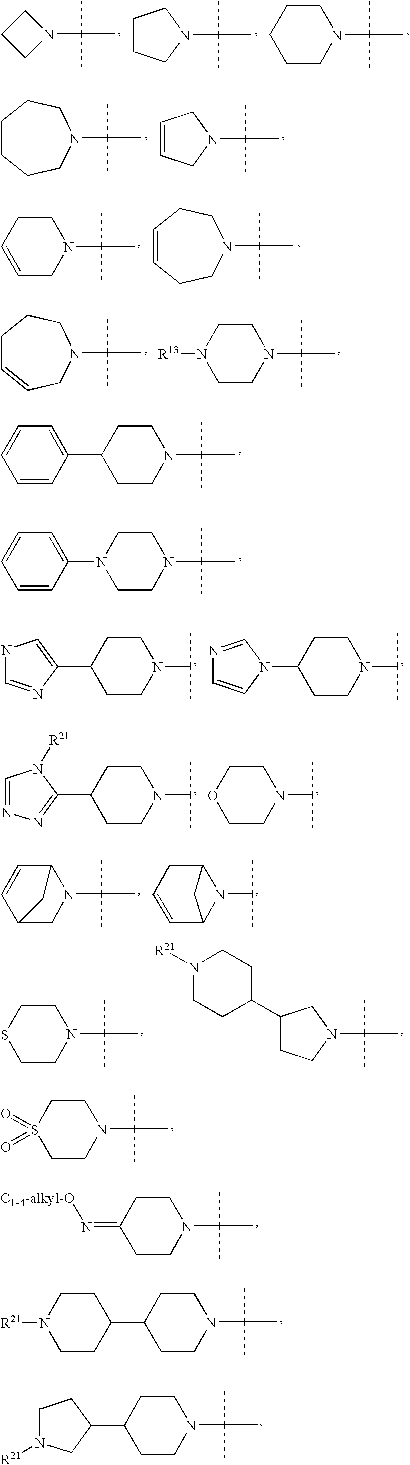

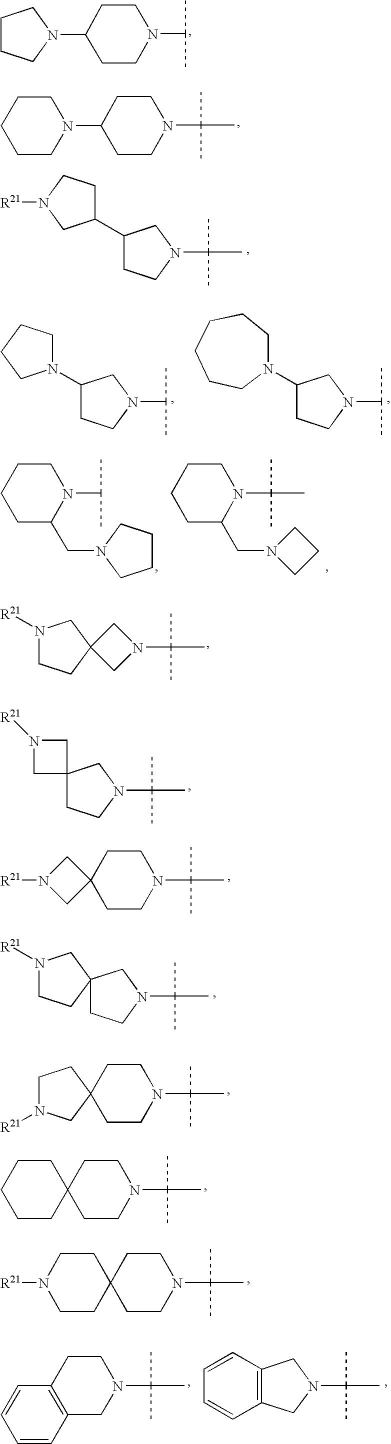

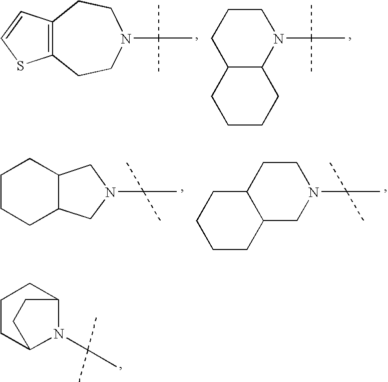

wherein one or more H atoms of the heterocycle formed by the group R¹R²N— may be replaced by identical or different groups R¹⁴, and
the heterocycle formed by the group R¹R²N— may be substituted by one or two, preferably one C₃₋₇-cycloalkyl group, while the cycloalkyl group may be mono- or polysubstituted by R²⁰, and
the ring attached to the heterocycle formed by the group R¹R²N— may be mono- or polysubstituted at one or more C atoms by R²⁰, or in the case of a phenyl ring may also additionally be monosubstituted by nitro and
wherein R¹³, R¹⁴, R²⁰, R²¹ have the meanings given hereinbefore and hereinafter.

If the heterocycle formed by the group R¹R²N— is substituted as specified by one or two cycloalkyl groups mono- or polysubstituted by R²⁰, the substituents R²⁰ independently of one another preferably denote C₁₋₄-alkyl, C₁₋₄-alkoxy-C₁₋₃-alkyl, hydroxy-C₁₋₃-alkyl, hydroxy, fluorine, chlorine, bromine or CF₃, particularly hydroxy.

Most particularly preferably the group

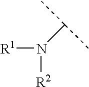

is defined according to one of the following partial formulae

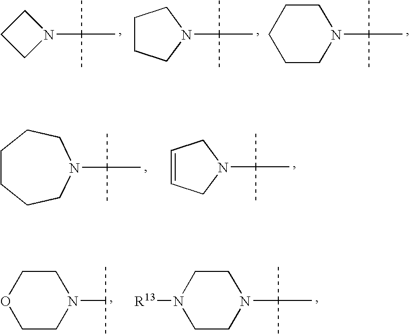

where R¹³ has the meanings given above and hereinafter, and
the heterocycle formed by the group R¹R²N— may be substituted by C₃₋₆-cycloalkyl, hydroxy-C₃₋₆-cycloalkyl or (hydroxy-C₃₋₆-cycloalkyl)-C₁₋₃-alkyl, and
the heterocycle formed by the group R¹R²N— may be mono-, di- or trisubstituted by identical or different groups R¹⁴.

The following partial formulae are most particularly preferred definitions of the heterocyclic group

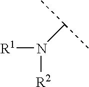

specified above:

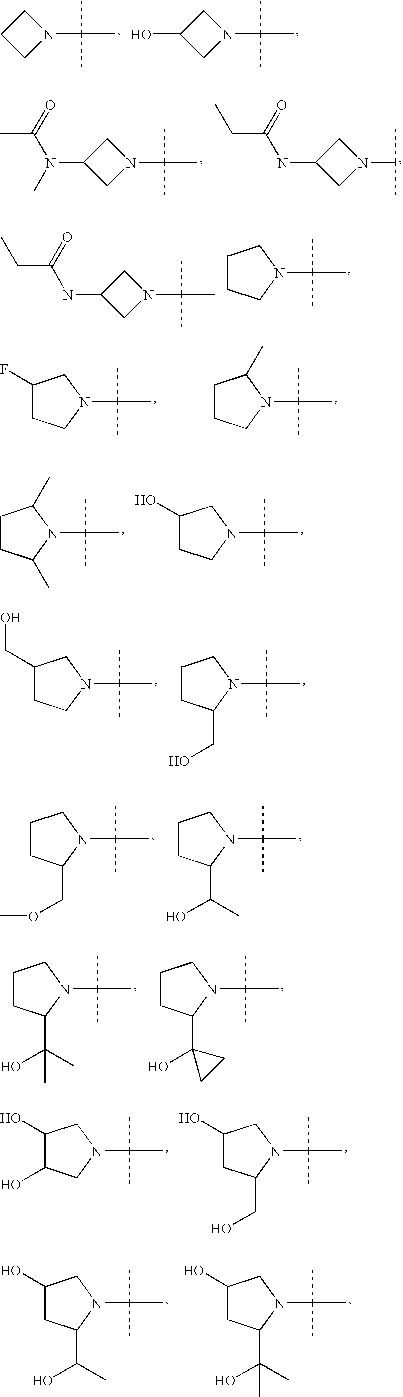

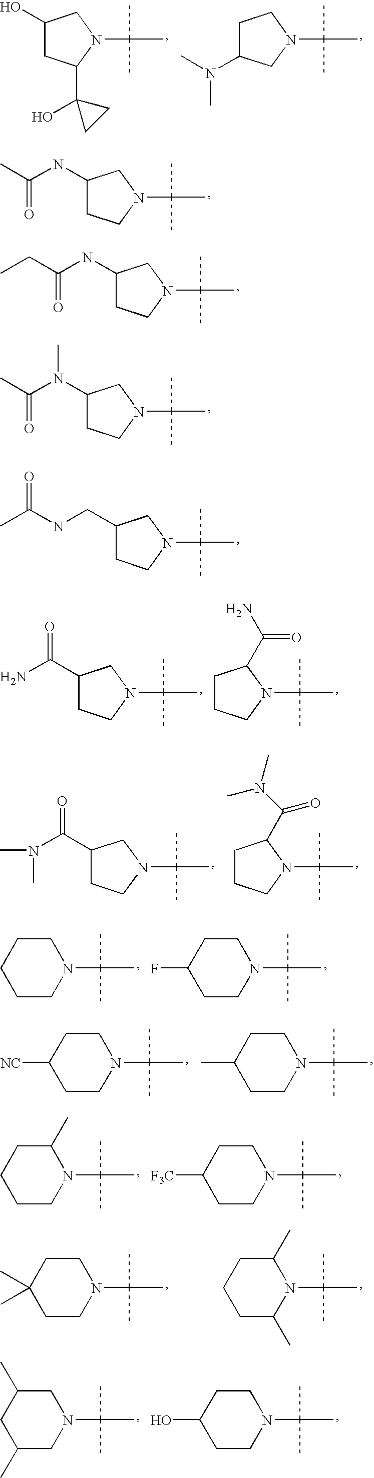

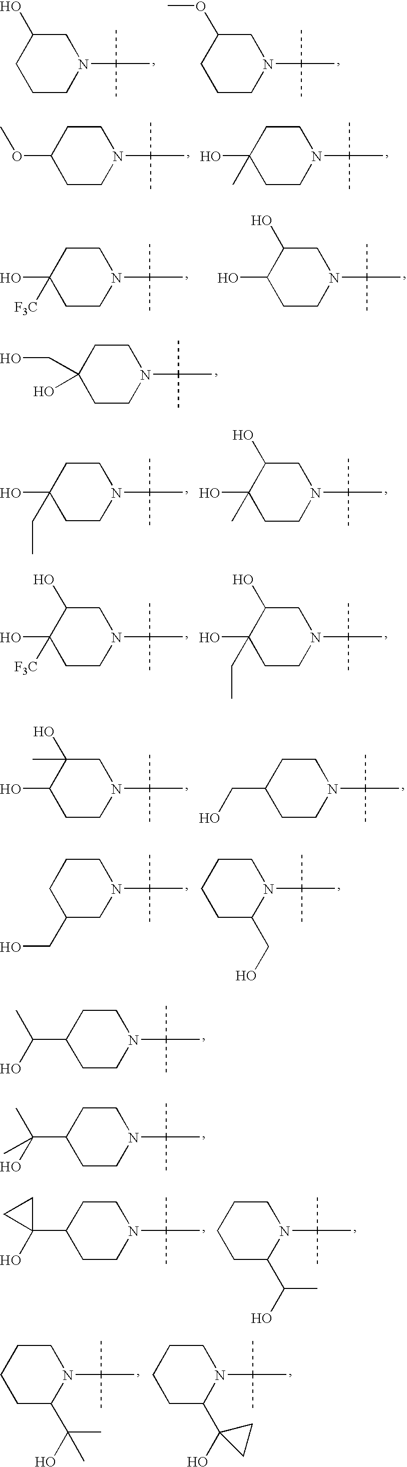

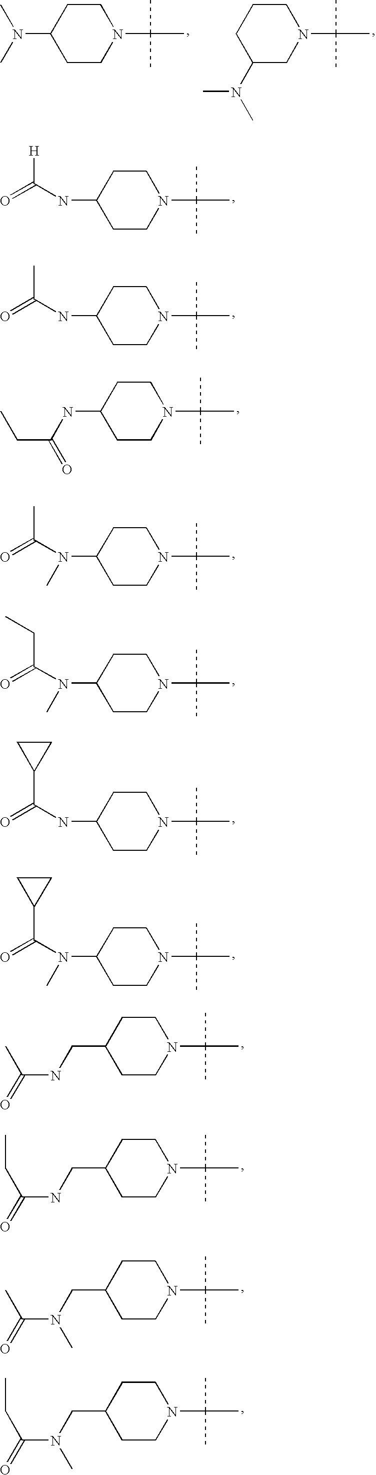

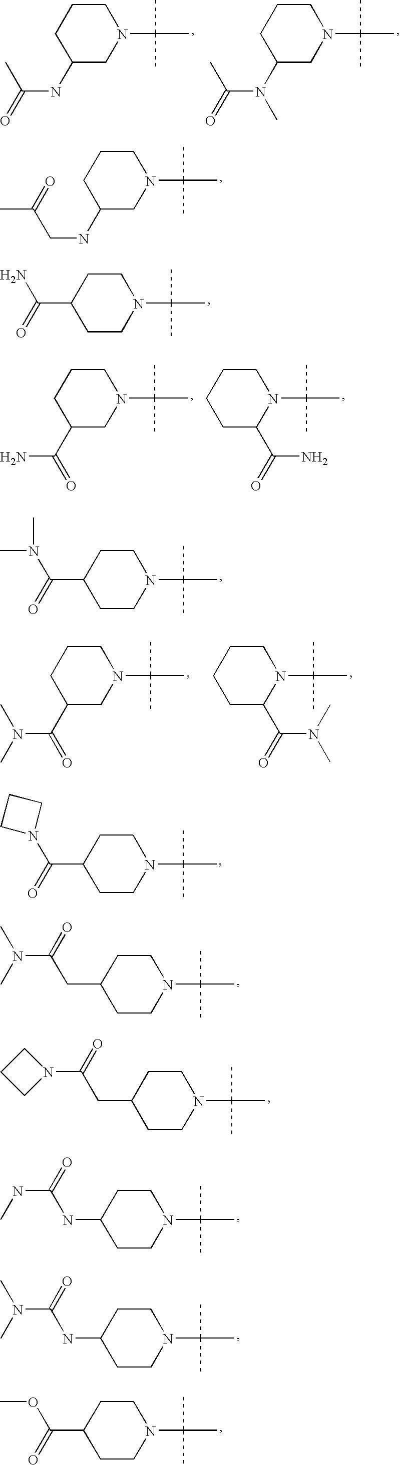

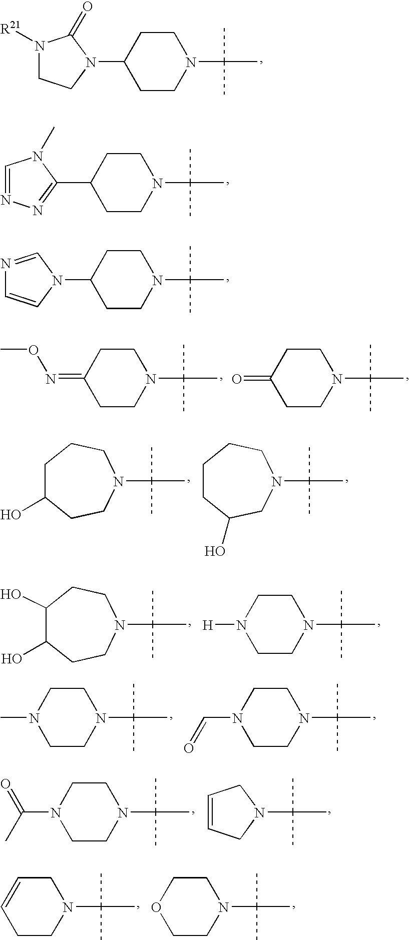

wherein the groups mentioned are not further substituted, or
wherein methyl or ethyl groups may be mono-, di- or trisubstituted by fluorine, and wherein one or more H atoms of the heterocycle formed by the group R¹R²N— which are bound to carbon may be substituted independently of one another by fluorine, chlorine, CN, CF₃, C₁₋₃-alkyl, hydroxy-C₁₋₃-alkyl, particularly C₁₋₃-alkyl or CF₃, preferably methyl, ethyl, CF₃.

Among the above-mentioned preferred and particularly preferred meanings of R¹R²N, the following definitions of the substituent R¹⁴ are preferred:
F, Cl, Br, cyano,
C₁₋₄-alkyl, C₂₋₄-alkenyl, C₂₋₄-alkynyl, C₃₋₇-cycloalkyl, C₃₋₇-cycloalkyl-C₁₋₃-alkyl,
hydroxy, hydroxy-C₁₋₃-alkyl, C₁₋₄-alkoxy, ω-(C₁₋₄-alkoxy)-C₁₋₃-alkyl,
C₁₋₄-alkyl-carbonyl, carboxy, C₁₋₄-alkoxycarbonyl, hydroxy-carbonyl-C₁₋₃-alkyl, C₁₋₄-alkoxy-carbonyl-C₁₋₃-alkyl,
formylamino, formyl-N(C₁₋₄-alkyl)-amino, formylamino-C₁₋₃-alkyl, formyl-N(C₁₋₄-alkyl)-amino-C₁₋₃-alkyl, C₁₋₄-alkyl-carbonylamino, C₁₋₄-alkyl-carbonyl-N—(C₁₋₄-alkyl)-amino, C₁₋₄-alkyl-carbonylamino-C₁₋₃-alkyl, C₁₋₄-alkyl-carbonyl-N—(C₁₋₄-alkyl)-amino-C₁₋₃-alkyl,
C₁₋₄-alkoxy-carbonylamino, C₁₋₄-alkoxy-carbonylamino-C₁₋₃-alkyl,
amino, C₁₋₄-alkyl-amino, C₃₋₇-cycloalkyl-amino, C₃₋₇-cycloalkyl-N—(C₁₋₄-alkyl)-amino, di-(C₁₋₄-alkyl)-amino, cyclo-C₃₋₆-alkyleneimino, amino-C₁₋₃-alkyl, C₁₋₄-alkyl-amino-C₁₋₃-alkyl, C₃₋₇-cycloalkyl-amino-C₁₋₃-alkyl, C₃₋₇-cycloalkyl-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl,
aminocarbonyl, C₁₋₄-alkyl-amino-carbonyl, C₃₋₇-cycloalkyl-amino-carbonyl, C₃₋₇-cycloalkyl-N—(C₁₋₄-alkyl)-amino-carbonyl, di-(C₁₋₄-alkyl)-amino-carbonyl, (aza-C₄₋₆-cycloalkyl)-carbonyl, aminocarbonyl-C₁₋₃-alkyl, C₁₋₄-alkyl-amino-carbonyl-C₁₋₃-alkyl, C₃₋₇-cycloalkyl-amino-carbonyl-C₁₋₃-alkyl, C₃₋₇-cycloalkyl-N—(C₁₋₄-alkyl)-amino-carbonyl-C₁₋₃-alkyl, di-(C₁₋₄-alkyl)-amino-carbonyl-C₁₋₃-alkyl, (aza-C₄₋₆-cycloalkyl)-carbonyl-C₁₋₃-alkyl,
C₁₋₄-alkyl-amino-carbonyl-amino-, di-(C₁₋₄-alkyl)-amino-carbonyl-amino-.

Particularly preferred meanings of the substituent R¹⁴ are selected from:
F, Cl, Br,
C₁₋₄-alkyl,
hydroxy, hydroxy-C₁₋₃-alkyl, C₁₋₄-alkoxy, ω-(C₁₋₄-alkoxy)-C₁₋₃-alkyl,
formylamino, formyl-N(C₁₋₄-alkyl)-amino, C₁₋₄-alkyl-carbonylamino, C₁₋₄-alkyl-carbonyl-N—(C₁₋₄-alkyl)-amino, C₁₋₄-alkyl-carbonylamino-C₁₋₃-alkyl, C₁₋₄-alkyl-carbonyl-N—(C₁₋₄-alkyl)-amino-C₁₋₃-alkyl,
di-(C₁₋₄-alkyl)-amino, amino-C₁₋₃-alkyl, C₁₋₄-alkyl-amino-C₁₋₃-alkyl, C₃₋₇-cycloalkyl-amino-C₁₋₃-alkyl, C₃₋₇-cycloalkyl-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl,
aminocarbonyl, C₁₋₄-alkyl-amino-carbonyl, di-(C₁₋₄-alkyl)-amino-carbonyl, (aza-C₄₋₆-cycloalkyl)-carbonyl, di-(C₁₋₄-alkyl)-amino-carbonyl-C₁₋₃-alkyl, (aza-C₄₋₆-cycloalkyl)-carbonyl-C₁₋₃-alkyl.

In the above-mentioned preferred meanings of R¹⁴ in each case one or more C atoms may additionally be mono- or polysubstituted by F and/or in each case one or two C atoms may independently of one another additionally be monosubstituted by Cl or Br. Thus, preferred meanings of R¹⁴ also include, for example, —CF₃, —OCF₃, CF₃—CO— and CF₃—CHOH—.

Most particularly preferred meanings of the substituent R¹⁴ are F, C₁₋₃-alkyl, hydroxy-C₁₋₃-alkyl, methoxy, methoxymethyl, hydroxy, aminocarbonyl, di(C₁₋₃-alkyl)amino, formylamino, formyl-N(C₁₋₃-alkyl)amino, C₁₋₃-alkylcarbonylamino, C₁₋₃-alkyl-carbonyl-N—(C₁₋₃-alkyl)-amino, C₁₋₃-alkylcarbonylamino-methyl, C₁₋₃-alkyl-carbonyl-N—(C₁₋₃-alkyl)-amino-methyl, C₁₋₃-alkylamino-carbonyl, di-(C₁₋₃-alkyl)-amino-carbonyl, C₁₋₃-alkyl-amino-carbonyl-methyl, di-(C₁₋₃-alkyl)-amino-carbonyl-methyl.

Examples of most preferred meanings of R¹⁴ are F, hydroxy, methyl, ethyl, CF₃, methoxy, hydroxymethyl, 2-hydroxyethyl, dimethylamino, formylamino, aminocarbonyl, methylaminocarbonyl, methylaminocarbonylmethyl, dimethylaminocarbonyl, dimethylaminocarbonylmethyl, methylcarbonylamino, methylcarbonylaminomethyl, ethylcarbonylamino, ethylcarbonylaminomethyl, methylcarbonyl-N-(methyl)-amino, methylcarbonyl-N-(methyl)-aminomethyl, ethylcarbonyl-N-(methyl)-amino, ethylcarbonyl-N-(methyl)-aminomethyl.

Preferably the group X denotes —CH₂—, —CH₂—CH₂—, —CH₂—CH₂—CH₂—, —CH₂—CH₂—O— or —CH₂—CH₂—NR^{N}—, wherein R^{N} is as defined hereinbefore, in particular wherein R^{N} denotes H oder methyl; most preferably —CH₂—, —CH₂—CH₂—, —CH₂—CH₂—O— or —CH₂—CH₂—NH—.

In case the substituent R² denotes an alkylene bridge which is linked to the group Y, then the group X preferably denotes —CH₂— or —CH₂—CH₂—.

The group Y is preferably selected from the group consisting of phenyl, pyridyl, pyridazinyl, pyrimidinyl, pyrazinyl, furyl and thiophenyl all of which may be mono- or polysubstituted by identical or different substituents R²⁰.

More preferably the group Y denotes phenyl, pyridyl, pyridazinyl and thiazolyl, which may be mono- or polysubstituted, in particular mono- or disubstituted by identical or different substituents R²⁰.

In particular the group Y denotes a group characterized by a subformula selected from

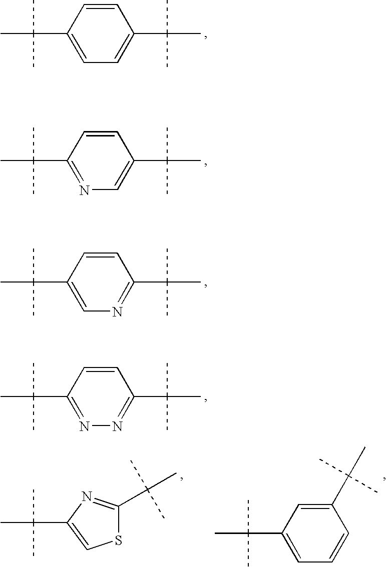

which may be mono- or disubstituted by identical or different substituents R²⁰.

Most preferably the group Y denotes a group characterized by a subformula selected from

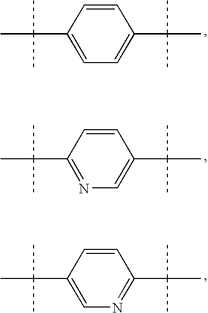

which may be mono- or disubstituted by identical or different substituents R²⁰.

Preferred substituents R²⁰ of the group Y are selected from halogen, C₁₋₃-alkyl, C₁₋₃-alkoxy, hydroxy and CF₃; in particular fluorine, chlorine, bromine or methyl.

In case the group R² denotes a C₁₋₃-alkylen bridge which is linked to the group Y, then the moiety of the formula R¹R²N—X—Y— preferably denotes

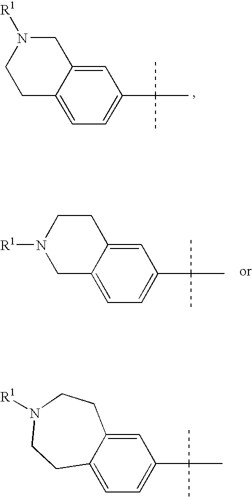

wherein R¹ is defined as hereinbefore, in particular R¹ denotes a group selected from C₁₋₄-alkyl-CO—, C₁₋₄-alkyl-O—CO—, (C₁₋₄-alkyl)NH—CO— or (C₁₋₄-alkyl)₂N—CO— wherein alkyl-groups may be mono- or polyfluorinated; most preferably R¹ denotes H or C₁₋₃-alkyl which may be mono- or polyfluorinated.

The group Z preferably denotes —CH₂—CH₂—, —C(═O)—CH₂—, —C(OH)H—CH₂—, —CH₂—C(═O)— or —CH₂—C(OH)H—, wherein one or more H-atoms may be replaced by F-atoms. In particular the group Z denotes —CH₂—CH₂— or —C(═O)—CH₂—, wherein one or more H-atoms may be replaced by F-atoms.

The group W is preferably selected from the group consisting of —CH₂—CH₂—, —CH₂—O—, —O—CH₂—, —O—CH(CH₃)—, —NR^{N}—CH₂—, wherein one or more H-atoms may be replaced by F-atoms, and wherein R^{N} is defined as hereinbefore, in particular wherein R^{N} denotes H oder methyl. Most preferably the group W denotes —O—CH₂— or —NH—CH₂—.

According to a first embodiment the group B is preferably selected from the group consisting of phenyl and 5- to 6-membered unsaturated or aromatic heterocyclic groups which contain 1 to 4 heteroatoms selected from N, O and S wherein the phenyl or heterocyclic group may be mono- or polysubstituted by identical or different substituents R²⁰.

More preferably the group B is selected from the group consisting of phenyl, pyridyl, pyridazinyl, pyrazinyl, pyrimidinyl, pyrrolyl, pyrazolyl, imidazolyl, triazolyl, tetrazolyl, furyl, thiophenyl and thiazolyl; in particular selected from phenyl, pyridyl, furyl and thiophenyl, wherein said group B may be mono- or polysubstituted, preferably mono- or disubstituted by identical or different substituents R²⁰.

Most preferably the group B denotes a group characterized by a subformula selected from

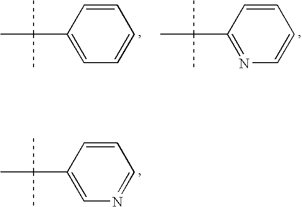

which may be mono- or polysubstituted, particularly mono- or disubstituted by identical or different substituents R²⁰.

In case the group B is a 6-membered ring, in particular a phenyl or pyridyl group, it is preferably unsubstituted or mono- or disubstituted by identical or different groups R²⁰, wherein the preferred position of a substituent is para with respect to the group W.

Preferred substituents R²⁰ of the group B are selected from halogen, hydroxy, nitro, C₁₋₃-alkyl, C₁₋₃-alkoxy, (C₁₋₃-alkyl)-carbonyl-, di-(C₁₋₃-alkyl)amino, aminocarbonyl, (C₁₋₃-alkyl)-carbonylamino and (C₁₋₃-alkyl)-sulfonylamino, wherein in each case one or more C atoms may additionally be mono- or polysubstituted by F. Preferred examples of fluorinated groups R²⁰ are CF₃ and —O—CF₃. Particularly preferred meanings of R²⁰ are fluorine, chlorine, bromine, methyl, methoxy and dimethylamino.

According to a second embodiment the group B preferably denotes C₂₋₆-alkyl, C₃₋₇-cycloalkyl or C₃₋₇-cycloalkyl-C₁₋₃-alkyl, wherein said alkyl-, cycloalkyl- or cycloalkylalkyl-groups may be mono- or poly-substituted independently of each other by R¹⁴; and where in cycloalkyl-rings one or two —CH₂-groups may be replaced independently of each other by —O—, —S—, —NR¹³— or —O(═O)—.

Even more preferably according to this second embodiment the group B preferably denotes C₂₋₆-alkyl, C₃₋₇-cycloalkyl or C₃₋₇-cycloalkyl-C₁₋₃-alkyl, wherein said alkyl-, cycloalkyl- or cycloalkylalkyl-groups may be mono- or poly-substituted independently of each other by F, Cl, Br, C₁₋₃-alkyl, CF₃, OH or C₁₋₃-alkoxy; and where in cycloalkyl-rings one —CH₂-group may be replaced independently of each other by —O—, —S—, —NR¹³— or —C(═O)—, wherein R¹³ is defined as hereinbefore or hereinafter, in particular wherein R¹³ denotes H or methyl.

Most preferably according to this second embodiment the group B denotes branched or linear C₂₋₆-alkyl, tetrahydrofuranyl or tetrahydropyranyl, in particular 2-methylprop-1-yl and tetrahydropyran-2-yl.

The following are preferred definitions of other substituents according to the invention:

The groups R^{N} independently of each other preferably denotes H, methyl, ethyl or formyl; most preferably H.

Preferably the substituent R¹³ has one of the meanings given for R¹⁶ or formyl. Particularly preferably R¹³ denotes H, C₁₋₄-alkyl, C₃₋₇-cycloalkyl, C₃₋₇-cycloalkyl-C₁₋₃-alkyl, ω-hydroxy-C₂₋₃-alkyl, ω-(C₁₋₄-alkoxy)-C₂₋₃-alkyl, formyl or (C₁₋₄-alkyl)-carbonyl. Most particularly preferably R¹³ denotes H, C₁₋₄-alkyl, formyl, methylcarbonyl or ethylcarbonyl. The alkyl groups mentioned hereinbefore may be monosubstituted by Cl or mono- or polysubstituted by F.

Preferred meanings of the substituent R¹⁵ are H, C₁₋₄-alkyl, C₃₋₇-cycloalkyl, C₃₋₇-cycloalkyl-C₁₋₃-alkyl, while, as defined hereinbefore, in each case one or more C atoms may additionally be mono- or polysubstituted by F and/or in each case one or two C atoms independently of one another may additionally be monosubstituted by Cl or Br. Particularly preferably R¹⁵ denotes H, CF₃, methyl, ethyl, propyl or butyl.

The substituent R¹⁶ preferably denotes H, C₁₋₄-alkyl, C₃₋₇-cycloalkyl, C₃₋₇-cycloalkyl-C₁₋₃-alkyl, ω-hydroxy-C₂₋₃-alkyl or ω-(C₁₋₄-alkoxy)-C₂₋₃-alkyl, while, as hereinbefore defined, in each case one or more C atoms may additionally be mono- or polysubstituted by F and/or in each case one or two C atoms independently of one another may additionally be monosubstituted by Cl or Br. More preferably R¹⁶ denotes H, CF₃, C₁₋₃-alkyl, C₃₋₆-cycloalkyl or C₃₋₆-cycloalkyl-C₁₋₃-alkyl; in particular H, methyl, ethyl, n-propyl and i-propyl.

Preferably the substituent R¹⁷ has one of the meanings given for R¹⁶ as being preferred or denotes C₁₋₄-alkylcarbonyl. Particularly preferably R¹⁷ denotes H, C₁₋₃-alkyl or C₁₋₃-alkylcarbonyl.

Preferably one or both of the substituents R¹⁸ and R¹⁹ independently of one another denotes hydrogen or C₁₋₄-alkyl, particularly hydrogen or methyl.

In general the substituent R²⁰ preferably denotes halogen, hydroxy, cyano, nitro, C₁₋₄-alkyl, C₁₋₄-alkoxy, hydroxy-C₁₋₄-alkyl, (C₁₋₃-alkyl)-carbonyl-, di-(C₁₋₃-alkyl)amino, aminocarbonyl, (C₁₋₃-alkyl)-carbonylamino, (C₁₋₃-alkyl)-sulfonylamino or R²²—C₁₋₃-alkyl, while, as hereinbefore defined, in each case one or more C atoms may additionally be mono- or polysubstituted by F and/or in each case one or two C atoms independently of one another may additionally be monosubstituted by Cl or Br.

The substituent R²² preferably denotes C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, amino, C₁₋₄-alkylamino, di-(C₁₋₄-alkyl)-amino, C₁₋₄-alkyl-carbonyl-amino, aminocarbonylamino or C₁₋₄-alkylaminocarbonyl-amino, while, as hereinbefore defined, in each case one or more C atoms may additionally be mono- or polysubstituted by F and/or in each case one or two C atoms independently of one another may additionally be monosubstituted by Cl or Br. Most particularly preferred meanings for R²² are C₁₋₄-alkoxy, C₁₋₄-alkylcarbonyl, amino, C₁₋₄-alkylamino, di-(C₁₋₄-alkyl)-amino, wherein one or more H atoms may be replaced by fluorine.

Preferred definitions of the group R²¹ are C₁₋₄-alkyl, C₁₋₄-alkylcarbonyl, C₁₋₄-alkylsulphonyl, —SO₂—NH₂, —SO₂—NH—C₁₋₃-alkyl, —SO₂—N(C₁₋₃-alkyl)₂ and cyclo-C₃₋₆-alkyleneimino-sulphonyl, while, as hereinbefore defined, in each case one or more C atoms may additionally be mono- or polysubstituted by F and/or in each case one or two C atoms independently of one another may additionally be monosubstituted by Cl or Br. Most particularly preferably R²¹ denotes C₁₋₄-alkyl or CF₃.

Cy preferably denotes a C₃₋₇-cycloalkyl, particularly a C₃₋₆-cycloalkyl group, a C₅₋₇-cycloalkenyl group, pyrrolidinyl, piperidinyl, piperazinyl, morpholinyl, thiomorpholinyl, aryl or heteroaryl, and the above-mentioned cyclic groups may be mono- or polysubstituted at one or more C atoms by identical or different groups R²⁰, or in the case of a phenyl group may also additionally be monosubstituted by nitro, and/or one or more NH groups may be substituted by R²¹. Most particularly preferred definitions of the group Cy are C₃₋₆-cycloalkyl, pyrrolidinyl and piperidinyl, which may be substituted as specified.

The term aryl preferably denotes phenyl or naphthyl, particularly phenyl.

The term heteroaryl preferably comprises pyridyl, pyridazinyl, thiophenyl, thiazolyl or furyl.

Preferred compounds according to the invention are those wherein one or more of the groups, radicals, substituents and/or indices have one of the meanings given hereinbefore as being preferred.

Preferred compounds according to the invention may be described by a general formula IIa to IIf:

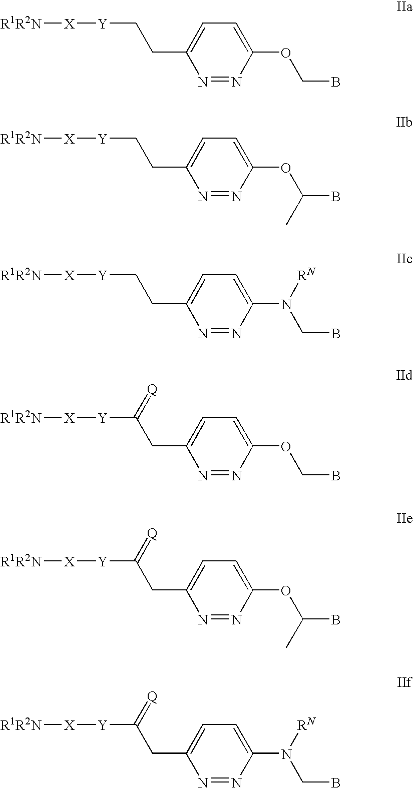

wherein
Q denotes O or CH₂; and
wherein the —CH₂—CH₂— and —C(═O)—CH₂— bridge linked to the group Y and to the pyridazine group may be mono- or polysubstituted with substituents independently from each other selected from F and C₁₋₃-alkyl; and
wherein the —CH₂—CH₂— bridge linked to the group Y and to the pyridazine group may be mono-substituted with hydroxy; and
wherein the groups R¹, R², R^{N}, X, Y and B are defined as hereinbefore and hereinafter; including the tautomers, the diastereomers, the enantiomers, the mixtures thereof and the salts thereof.

Particularly preferred compounds according to the invention may be described by a general formula IIIa to IIIm:

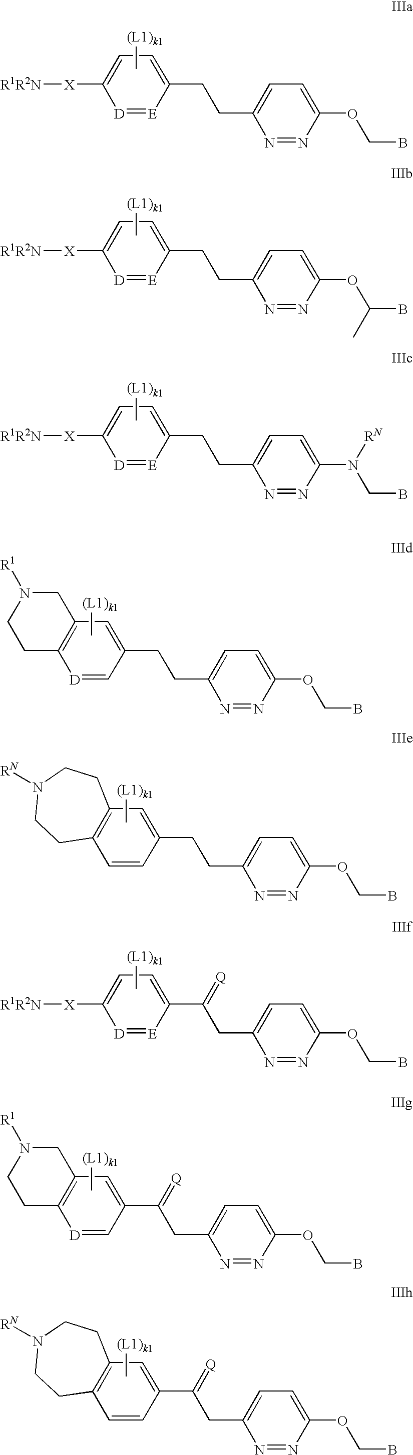

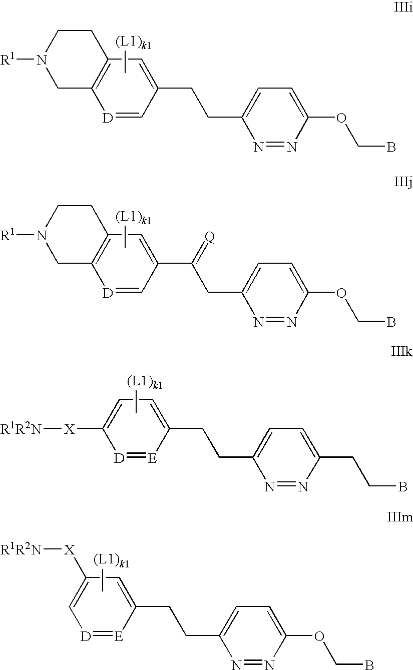

wherein
D, E independently of each other denote CH or N, wherein CH may be substituted with L1; in particular wherein D and E denote CH which may be substituted with L1 or wherein D or E denotes N and the other of D and E denotes CH which may be substituted with L1; and
L1 are independently of one another selected from the meanings of R²⁰ as defined hereinbefore, in particular of the meanings of R²⁰ as a substituent of the group Y as defined hereinbefore; and
k1 denotes 0, 1 or 2; in particular 0 or 1; and
Q denotes O or CH₂; and
   wherein the —CH₂—CH₂— and —O(═O)—CH₂— bridge linked to the group Y being

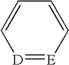

or phenyl and to the pyridazine group may be mono- or polysubstituted with substituents independently from each other selected from F and C₁₋₃-alkyl; and
wherein the —CH₂—CH₂— bridge linked to the group Y and to the pyridazine group may be mono-substituted with hydroxy; and
wherein the groups R¹, R², R^{N}, X and B are defined as hereinbefore and hereinafter; including the tautomers, the diastereomers, the enantiomers, the mixtures thereof and the salts thereof.

In the above formulae the group B preferably denotes phenyl or pyridyl which may be mono- or polysubstituted, particularly mono- or disubstituted by identical or different substituents R²⁰ as defined hereinbefore.

In particular in the formulae IIa to IIf and IIIa to IIIm the following definitions are preferred:
R¹, R² independently of one another denote C₁₋₄-alkyl, hydroxy-C₁₋₄-alkyl, C₃₋₅-alkenyl, C₃₋₅-alkynyl, C₃₋₇-cycloalkyl, hydroxy-C₃₋₇-cycloalkyl, dihydroxy-C₃₋₆-alkyl, C₃₋₇-cycloalkyl-C₁₋₃-alkyl, tetrahydropyran-3-yl, tetrahydropyran-4-yl, tetrahydrofuran-2-ylmethyl, tetrahydrofuran-3-ylmethyl, (hydroxy-C₃₋₇-cycloalkyl)-C₁₋₃-alkyl, C₁₋₄-alkoxy-C₂₋₃-alkyl, hydroxy-C₁₋₄-alkoxy-C₂₋₃-alkyl, C₁₋₄-alkoxy-C₁₋₄-alkoxy-C₂₋₃-alkyl, di-(C₁₋₃-alkyl)amino-C₂₋₃-alkyl, pyrrolidin-N-yl-C₂₋₃-alkyl, piperidin-N-yl-C₂₋₃-alkyl, pyridylmethyl, pyrazolylmethyl, thiazolylmethyl and imidazolylmethyl, while an alkyl, cycloalkyl or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl or CN; and the above-mentioned pyridyl, pyrazolyl, thiazolyl or imidazolyl group may be mono- or polysubstituted with a substituent independently of each other selected from F, Cl, Br, I, cyano, C₁₋₃-alkyl, C₁₋₃-alkoxy, trifluoromethyl, hydroxy, amino, acetylamino, aminocarbonyl; and one or both, preferably one of the groups R¹ and R² may also represent H; or
R¹, R² are joined together and form together with the N atom to which they are bound a heterocyclic group which is selected from azetidine, pyrrolidine, piperidine, azepan, 2,5-dihydro-1H-pyrrole, 1,2,3,6-tetrahydro-pyridine, 2,3,4,7-tetrahydro-1H-azepine, 2,3,6,7-tetrahydro-1H-azepine, piperazine in which the free imine function is substituted by R¹³, piperidin-4-one, morpholine, thiomorpholine, 1-oxo-thiomorpholin-4-yl, 1,1-dioxo-thiomorpholin-4-yl, 4-C₁₋₄-alkoxy-imino-piperidin-1-yl and 4-hydroxyimino-piperidin-1-yl;
   wherein one or more H atoms may be replaced by identical or different groups R¹⁴, and
   the heterocyclic group defined hereinbefore may be substituted via a single bond by a carbo- or heterocyclic group Cy, while Cy is selected from the group comprising C₃₋₇-cycloalkyl, cyclo-C₃₋₆-alkyleneimino, imidazol, triazol, while Cy may be mono- or polysubstituted by identical or different groups R²⁰, wherein R²⁰ is defined as hereinbefore and is preferably selected from fluorine, CF₃, C₁₋₃-alkyl, hydroxy-C₁₋₃-alkyl and hydroxy, and
R¹⁴ is independently selected from
   —F, Cl, Br, cyano,
   C₁₋₄-alkyl, C₂₋₄-alkenyl, C₂₋₄-alkynyl, C₃₋₇-cycloalkyl, C₃₋₇-cycloalkyl-C₁₋₃-alkyl,
   hydroxy, hydroxy-C₁₋₃-alkyl, C₁₋₄-alkoxy, ω-(C₁₋₄-alkoxy)-C₁₋₃-alkyl,
   C₁₋₄-alkyl-carbonyl, carboxy, C₁₋₄-alkoxycarbonyl, hydroxy-carbonyl-C₁₋₃-alkyl, C₁₋₄-alkoxycarbonyl-C₁₋₃-alkyl,
   formylamino, N-formyl-N(C₁₋₄-alkyl)-amino, formylamino-C₁₋₃-alkyl, N-formyl-N(C₁₋₄-alkyl)-amino-C₁₋₃-alkyl, C₁₋₄-alkyl-carbonylamino, N—(C₁₋₄-alkyl-carbonyl)-N—(C₁₋₄-alkyl)-amino, C₁₋₄-alkyl-carbonylamino-C₁₋₃-alkyl, N—(C₁₋₄-alkyl-carbonyl)-N—(C₁₋₄-alkyl)-amino-C₁₋₃-alkyl,
   C₁₋₄-alkoxy-carbonylamino, C₁₋₄-alkoxy-carbonylamino-C₁₋₃-alkyl,
   amino, C₁₋₄-alkyl-amino, C₃₋₇-cycloalkyl-amino, N—(C₃₋₇-cycloalkyl)-N—(C₁₋₄-alkyl)-amino, di-(C₁₋₄-alkyl)-amino, cyclo-C₃₋₆-alkyleneimino, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl,
   aminocarbonyl, C₁₋₄-alkyl-amino-carbonyl, C₃₋₇-cycloalkyl-amino-carbonyl, N—(C₃₋₇-cycloalkyl)-N—(C₁₋₄-alkyl)-amino-carbonyl, di-(C₁₋₄-alkyl)-amino-carbonyl, (aza-C₄₋₆-cycloalkyl)-carbonyl, aminocarbonyl-C₁₋₃-alkyl, C₁₋₄-alkyl-amino-carbonyl-C₁₋₃-alkyl, C₃₋₇-cycloalkyl-amino-carbonyl-C₁₋₃-alkyl, N—(C₃₋₇-cycloalkyl)-N—(C₁₋₄-alkyl)-amino-carbonyl-C₁₋₃-alkyl, di-(C₁₋₄-alkyl)-amino-carbonyl-C₁₋₃-alkyl, (aza-C₄₋₆-cycloalkyl)-carbonyl-C₁₋₃-alkyl,
   C₁₋₄-alkyl-amino-carbonyl-amino-, di-(C₁₋₄-alkyl)-amino-carbonyl-amino-;
   while in the above-mentioned meanings in each case one or more C atoms may additionally be mono- or polysubstituted by F and/or in each case one or two C atoms independently of one another may additionally be monosubstituted by Cl or Br; and
X denotes —CH₂—, —CH₂—CH₂—, —CH₂—CH₂—CH₂—, —CH₂—CH₂—O— or —CH₂—CH₂—NR^{N}—; in particular —CH₂—, —CH₂—CH₂—, —CH₂—CH₂—O— or —CH₂—CH₂—NH—; and
B selected from the group consisting of phenyl, pyridyl, pyridazinyl, pyrazinyl, pyrimidinyl, pyrrolyl, pyrazolyl, imidazolyl, triazolyl, tetrazolyl, furyl, thiophenyl and thiazolyl; in particular selected from phenyl and pyridyl, wherein said group B may be mono- or polysubstituted, preferably mono- or disubstituted by identical or different substituents R²⁰ as defined hereinbefore or hereinafter; or
   the group B denotes C₂₋₆-alkyl, C₃₋₇-cycloalkyl or C₃₋₇-cycloalkyl-C₁₋₃-alkyl, wherein said alkyl-, cycloalkyl- or cycloalkylalkyl-groups may be mono- or poly-substituted independently of each other by F, Cl, Br, C₁₋₃-alkyl, CF₃, OH or C₁₋₃-alkoxy; and where in cycloalkyl-rings one —CH₂-group may be replaced independently of each other by —O—, —S—, —NR¹³— or —C(═O)—, wherein R¹³ is defined as hereinbefore or hereinafter, in particular wherein R¹³ denotes H or methyl; and
W denotes —CH₂—O—, —O—CH₂—, —O—CH(CH₃)— and —NR^{N}—CH₂—; most preferably —O—CH₂— or —NH—CH₂—; and
R²⁰ independently of one another denote halogen, hydroxy, nitro, C₁₋₃-alkyl, C₁₋₃-alkoxy, (C₁₋₃-alkyl)-carbonyl-, di-(C₁₋₃-alkyl)amino, aminocarbonyl, (C₁₋₃-alkyl)-carbonylamino and (C₁₋₃-alkyl)-sulfonylamino, wherein in each case one or more C atoms may additionally be mono- or polysubstituted by F; in particular fluorine, chlorine, bromine, methyl, methoxy and dimethylamino; and
R^{N} independently of each other denotes H, C₁₋₃-alkyl or formyl; more preferably H or methyl; and
L1 halogen, C₁₋₃-alkyl, C₁₋₃-alkoxy, hydroxy and CF₃; and
k1 is 0 or 1.

The compounds listed in the experimental section, including the tautomers, the diastereomers, the enantiomers, the mixtures thereof and the salts thereof, are preferred according to the invention.

Some expressions used hereinbefore and below to describe the compounds according to the invention will now be defined more fully.

The term halogen denotes an atom selected from among F, Cl, Br and I, particularly F, Cl and Br.

The term C₁₋ₙ-alkyl, where n has a value of 3 to 8, denotes a saturated, branched or unbranched hydrocarbon group with 1 to n C atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term C₁₋ₙ-alkylene, where n may have a value of 1 to 8, denotes a saturated, branched or unbranched hydrocarbon bridge with 1 to n C atoms. Examples of such groups include methylene (—CH₂—), ethylene (—CH₂—CH₂—), 1-methyl-ethylene (—CH(CH₃)—CH₂—), 1,1-dimethyl-ethylene (—C(CH₃)₂—CH₂—), n-prop-1,3-ylene (—CH₂—CH₂—CH₂—), 1-methylprop-1,3-ylene (—CH(CH₃)—CH₂—CH₂—), 2-methylprop-1,3-ylene (—CH₂—CH(CH₃)—CH₂—), etc., as well as the corresponding mirror-symmetrical forms.

The term C₂₋ₙ-alkenyl, where n has a value of 3 to 6, denotes a branched or unbranched hydrocarbon group with 2 to n C atoms and at least one C═C-double bond. Examples of such groups include vinyl, 1-propenyl, 2-propenyl, iso-propenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 4-hexenyl, 5-hexenyl etc.

The term C₂₋ₙ-alkynyl, where n has a value of 3 to 6, denotes a branched or unbranched hydrocarbon group with 2 to n C atoms and a CC triple bond. Examples of such groups include ethynyl, 1-propynyl, 2-propynyl, iso-propynyl, 1-butynyl, 2-butynyl, 3-butynyl, 2-methyl-1-propynyl, 1-pentynyl, 2-pentynyl, 3-pentynyl, 4-pentynyl, 3-methyl-2-butynyl, 1-hexynyl, 2-hexynyl, 3-hexynyl, 4-hexynyl, 5-hexynyl etc.

The term C₁₋ₙ-alkoxy denotes a C₁₋ₙ-alkyl-O— group, wherein C₁₋ₙ-alkyl is defined as above. Examples of such groups include methoxy, ethoxy, n-propoxy, iso-propoxy, n-butoxy, iso-butoxy, sec-butoxy, tert-butoxy, n-pentoxy, iso-pentoxy, neo-pentoxy, tert-pentoxy, n-hexoxy, iso-hexoxy etc.

The term C₁₋ₙ-alkylthio denotes a C₁₋ₙ-alkyl-S— group, wherein C₁₋ₙ-alkyl is defined as above. Examples of such groups include methylthio, ethylthio, n-propylthio, iso-propylthio, n-butylthio, iso-butylthio, sec-butylthio, tert-butylthio, n-pentylthio, iso-pentylthio, neo-pentylthio, tert-pentylthio, n-hexylthio, iso-hexylthio, etc.

The term C₁₋ₙ-alkylcarbonyl denotes a C₁₋ₙ-alkyl —C(═O)— group, wherein C₁₋ₙ-alkyl is defined as above. Examples of such groups include methylcarbonyl, ethylcarbonyl, n-propylcarbonyl, iso-propylcarbonyl, n-butylcarbonyl, iso-butylcarbonyl, sec-butylcarbonyl, tert-butylcarbonyl, n-pentylcarbonyl, iso-pentylcarbonyl, neo-pentylcarbonyl, tert-pentylcarbonyl, n-hexylcarbonyl, iso-hexylcarbonyl, etc.

The term C₃₋ₙ-cycloalkyl denotes a saturated mono-, bi-, tri- or spirocarbocyclic, preferably monocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, bicyclo[3,2,1]octyl, spiro[4,5]decyl, norpinyl, norbonyl, norcaryl, adamantyl, etc.

The term C₅₋ₙ-cycloalkenyl denotes a monounsaturated mono-, bi-, tri- or spirocarbocyclic, preferably monocarboxylic group with 5 to n C atoms. Examples of such groups include cyclopentenyl, cyclohexenyl, cycloheptenyl, cyclooctenyl, cyclononenyl, etc.

The term C₃₋ₙ-cycloalkylcarbonyl denotes a C₃₋ₙ-cycloalkyl-C(═O) group, wherein C₃₋ₙ-cycloalkyl is as hereinbefore defined.

The term aryl denotes a carbocyclic, aromatic ring system, such as for example phenyl, biphenyl, naphthyl, anthracenyl, phenanthrenyl, fluorenyl, indenyl, pentalenyl, azulenyl, biphenylenyl, etc. A particularly preferred meaning of “aryl” is phenyl.

The term cyclo-C₃₋₆-alkyleneimino denotes a 4- to 7-membered ring which comprises 3 to 6 methylene units as well as an imino group, while the bond to the residue of the molecule is made via the imino group.

The term cyclo-C₃₋₆-alkyleneimino-carbonyl denotes a cyclo-C₃₋₆-alkyleneimino ring as hereinbefore defined which is linked to a carbonyl group via the imino group.

The term heteroaryl used in this application denotes a heterocyclic, aromatic ring system which comprises in addition to at least one C atom one or more heteroatoms selected from N, O and/or S. Examples of such groups are furanyl, thiophenyl, pyrrolyl, oxazolyl, thiazolyl, imidazolyl, isoxazolyl, isothiazolyl, 1,2,3-triazolyl, 1,3,5-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinozilinyl, quinolinyl, isoquinolinyl, quinoxalinyl, naphthyridinyl, pteridinyl, carbazolyl, azepinyl, diazepinyl, acridinyl, etc. The term heteroaryl also comprises the partially hydrogenated heterocyclic, aromatic ring systems, particularly those listed above. Examples of such partially hydrogenated ring systems are 2,3-dihydrobenzofuranyl, pyrrolinyl, pyrazolinyl, indolinyl, oxazolidinyl, oxazolinyl, oxazepinyl, etc. Particularly preferably heteroaryl denotes a heteroaromatic mono- or bicyclic ring system.

Terms such as C₃₋₇-cycloalkyl-C₁₋ₙ-alkyl, heteroaryl-C₁₋ₙ-alkyl, etc. refer to C₁₋ₙ-alkyl, as defined above, which is substituted with a C₃₋₇-cycloalkyl, aryl or heteroaryl group.

Many of the terms given above may be used repeatedly in the definition of a formula or group and in each case have one of the meanings given above, independently of one another. Thus, for example, in the group di-C₁₋₄-alkyl-amino, the two alkyl groups may have the same or different meanings.

The term “unsaturated”, for example in “unsaturated carbocyclic group” or “unsaturated heterocyclic group”, as used particularly in the definition of the group Cy, comprises in addition to the mono- or polyunsaturated groups, the corresponding, totally unsaturated groups, but particularly the mono- and diunsaturated groups.

The term “optionally substituted” used in this application indicates that the group thus designated is either unsubstituted or mono- or polysubstituted by the substituents specified. If the group in question is polysubstituted, the substituents may be identical or different.

The style used hereinbefore and hereinafter, according to which in a cyclic group a bond of a substituent is shown towards the centre of this cyclic group, indicates unless otherwise stated that this substituent may be bound to any free position of the cyclic group carrying an H atom. Thus in the example

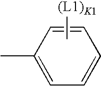

the substituent L1 where k1=1 may be bound to any of the free positions of the phenyl ring; where k1=2 selected substituents L1 may independently of one another be bound to different free positions of the phenyl ring.

The H atom of any carboxy group present or an H atom bound to an N atom (imino or amino group) may in each case be replaced by a group which can be cleaved in vivo. By a group which can be cleaved in vivo from an N atom is meant, for example, a hydroxy group, an acyl group such as the benzoyl or pyridinoyl group or a C₁₋₁₆-alkanoyl group such as the formyl, acetyl, propionyl, butanoyl, pentanoyl or hexanoyl group, an allyloxycarbonyl group, a C₁₋₁₆-alkoxycarbonyl group such as the methoxycarbonyl, ethoxycarbonyl, propoxycarbonyl, isopropoxycarbonyl, butoxycarbonyl, tert.butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulphonyl-C₂₋₄-alkoxycarbonyl, C₁₋₃-alkoxy-C₂₋₄-alkoxy-C₂₋₄-alkoxycarbonyl or RₑCO—O—(R_{f}CR_{g})—O—CO— group wherein
Rₑ denotes a C₁₋₈-alkyl, C₆₋₇-cycloalkyl, phenyl or phenyl-C₁₋₃-alkyl group,
R_{f} denotes a hydrogen atom, a C₁₋₃-alkyl, C₆₋₇-cycloalkyl or phenyl group and
R_{g} denotes a hydrogen atom, a C₁₋₃-alkyl or RₑCO—O—(R_{f}CRₕ)—O group wherein Rₑ and R_{f} are as hereinbefore defined and Rₕ is a hydrogen atom or a C₁₋₃-alkyl group,
   while the phthalimido group is an additional possibility for an amino group, and the above-mentioned ester groups may also be used as a group which can be converted in vivo into a carboxy group.

The residues and substituents described above may be mono- or polysubstituted by fluorine as described. Preferred fluorinated alkyl groups are fluoromethyl, difluoromethyl and trifluoromethyl. Preferred fluorinated alkoxy groups are fluoromethoxy, difluoromethoxy and trifluoromethoxy. Preferred fluorinated alkylsulphinyl and alkylsulphonyl groups are trifluoromethylsulphinyl and trifluoromethylsulphonyl.

The compounds of general formula I according to the invention may have acid groups, predominantly carboxyl groups, and/or basic groups such as e.g. amino functions. Compounds of general formula I may therefore be present as internal salts, as salts with pharmaceutically useable inorganic acids such as hydrochloric acid, sulphuric acid, phosphoric acid, sulphonic acid or organic acids (such as for example maleic acid, fumaric acid, citric acid, tartaric acid or acetic acid) or as salts with pharmaceutically useable bases such as alkali or alkaline earth metal hydroxides or carbonates, zinc or ammonium hydroxides or organic amines such as e.g. diethylamine, triethylamine, triethanolamine inter alia.

The compounds according to the invention may be obtained using methods of synthesis which are known to the one skilled in the art and described in the literature of organic synthesis. Preferably the compounds are obtained analogously to the methods of preparation explained more fully hereinafter, in particular as described in the experimental section.

Compounds of the general formula IIIa can be prepared depending on the nature of the linker group X and the groups D and E by the following methods:
1. Reductive amination of the precursor A-1 with hydride donors such as triacetoxyborohydride (either free or resin bound), the appropriate amines and acid like acetic acid in solvents like THF, preferably at room temperature.
2. Reaction of the precursor A-2, A-5 or A-6 with an appropriate amine and a base such as potassium carbonate in solvents such as acetone at temperatures between room temperature and 120° C.
3. Reduction of the precursor A-3 or A-4 with hydrogen in the presence of a catalyst such as Raney-Nickel in a solvent such as DMF or methanol at temperatures between room temperature and 120° C.

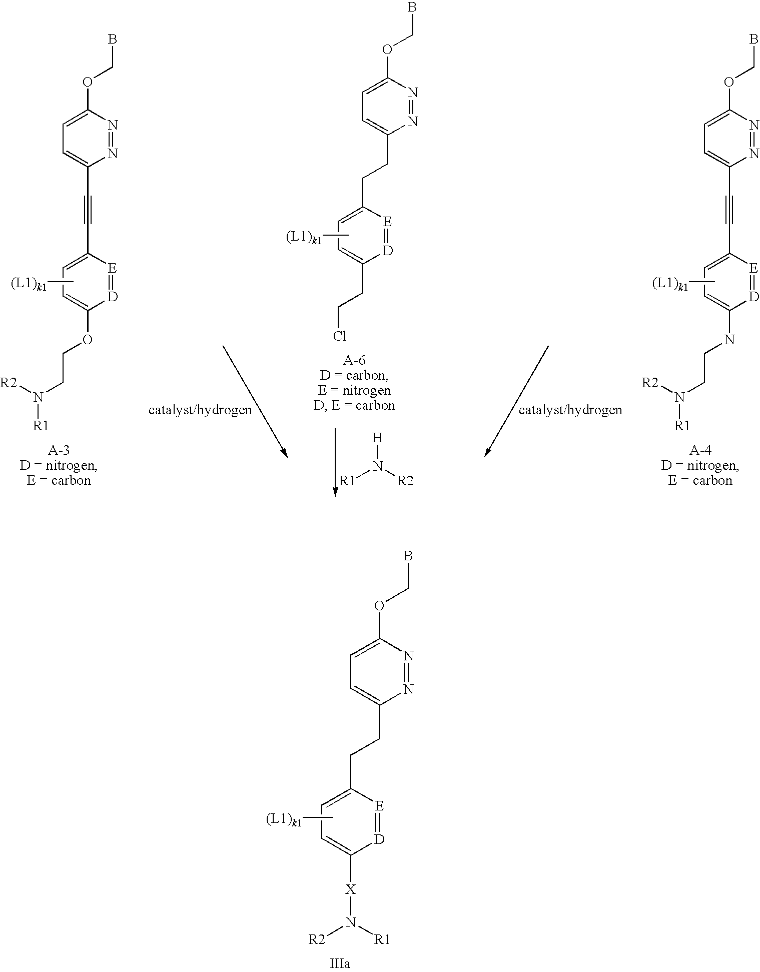

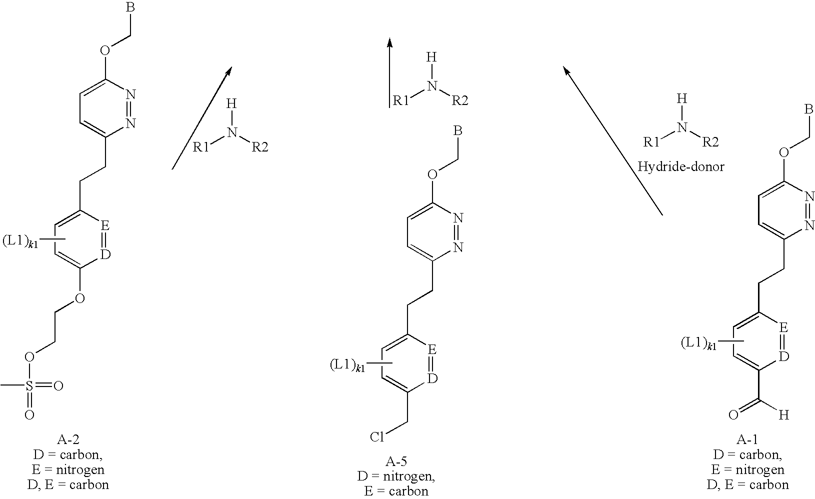

The synthesis of the precursor A-1 is outlined below.

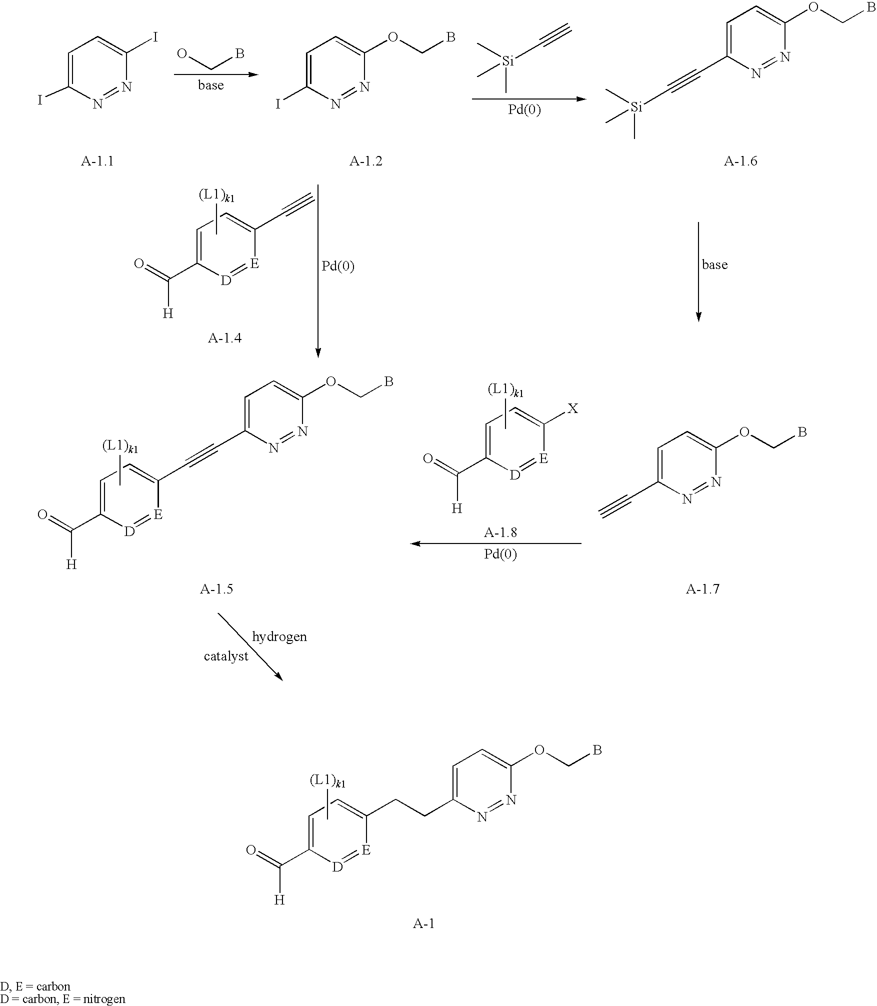

Pyridazine derivative A1.2 is obtained by reaction of the sodium salt of an appropriate alcohol with A1.1 in solvents such as toluene at temperatures between 0° C. and 120° C. A1.5 can be obtained directly by Sonogashira reaction of A1.2 and A1.4 in a solvent such as THF at temperatures between 0° C. and 120° C. Alternatively A1.5 can be synthesized by Sonogashira reaction of A1.2 with a protected acetylene derivative leading to A1.6, followed by deprotection using a base such as sodium hydroxide resulting in the formation of A1.7. Subsequent Sonogashira reaction of A1.7 with A1.8 gives A1.5. A1.5 can be reduced catalytically by catalysts such as Raney-Nickel in solvents like DMF under hydrogen atmosphere to A-1.

The synthesis of the precursor A-2 is outlined below.

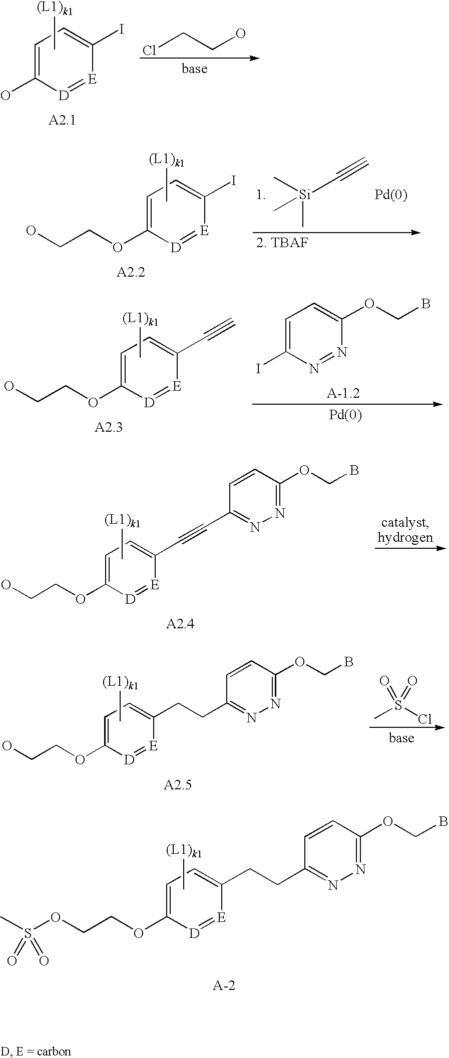

The phenol derivative A2.1 is reacted with 2-chloro-ethanol in the presence of a base like potassium carbonate in solvents such as DMF to give A2.2. Sonogashira reaction of A2.2 with a protected acetylene, followed by deprotection with for example tetrabutyl ammonium fluoride gives A2.3. Compound A2.4 is formed by Sonogashira reaction of A2.3 with A1.2. Catalytic reduction of A2.4 by catalysts such as Raney-Nickel in solvents like DMF under hydrogen atmosphere gives A2.5, which is converted to A-2 via reaction with methane sulfonyl chloride in solvents such as methylene chloride in the presence of a base such as triethylamine at temperatures between 0° C. and 120° C.

The synthesis of the precursor A-3 is outlined below.

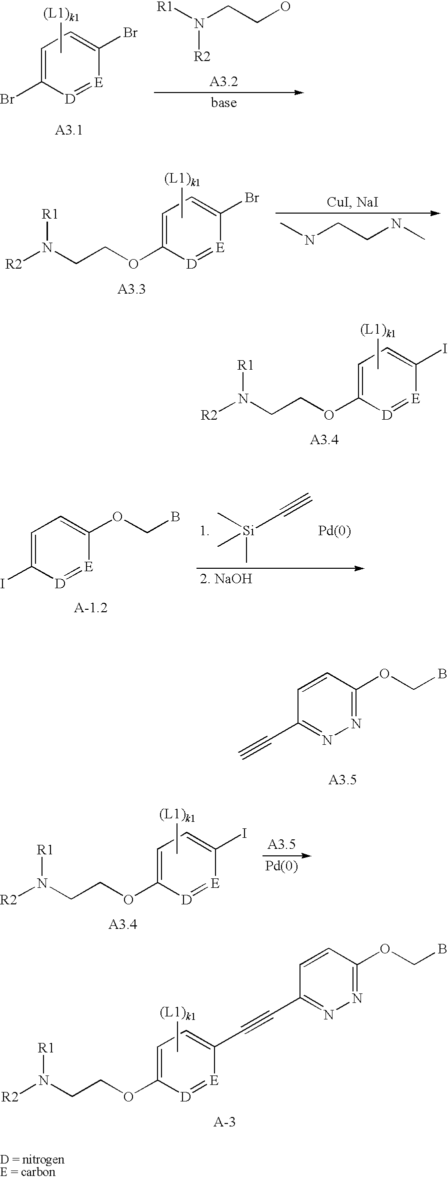

Heterocyclic dibromo derivative A3.1 is reacted with A3.2 with the help of a base like sodium hydride in solvents such as DMF to give A3.3. A3.3 is converted to the iodo compound A3.4 by reaction with sodium iodide, copper iodide and N,N′-dimethylethylenediamine in a solvent such as dioxane at temperatures between 0° C. and 120° C. Sonogashira reaction of A1.2 with a protected acetylene, followed by deprotection with for example tetrabutyl ammonium fluoride or sodium hydroxide gives A3.5. A-3 is formed by Sonogashira reaction of A3.4 with A3.5.

The synthesis of the precursor A-4 is outlined below.

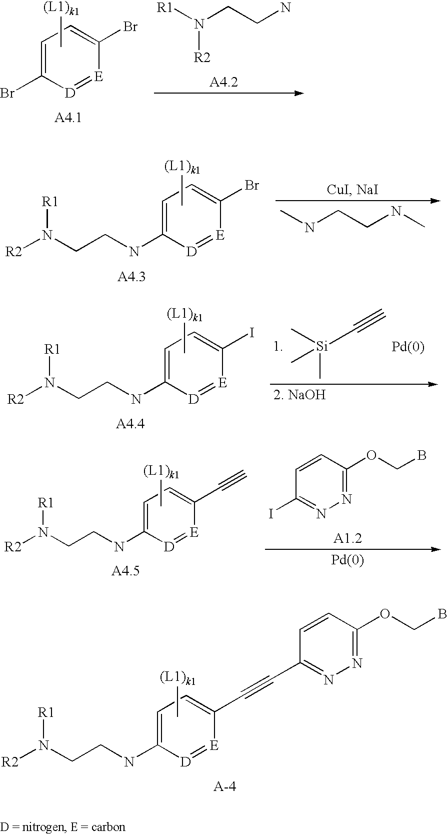

Heterocyclic dibromo derivative A4.1 is reacted with A4.2 at temperatures between 0° C. and 120° C. to give A4.3. The compound A4.3 is converted to the iodo compound A4.4 by reaction with sodium iodide, copper iodide and N,N′-dimethylethylenediamine in a solvent such as dioxane at temperatures between 0° C. and 120° C. Sonogashira reaction of A4.4 with a protected acetylene, followed by deprotection with for example tetrabutyl ammonium fluoride or sodium hydroxide gives A4.5. The compound A-4 is formed by Sonogashira reaction of A4.5 with A1.2.

The synthesis of the precursor A-5 is outlined below.

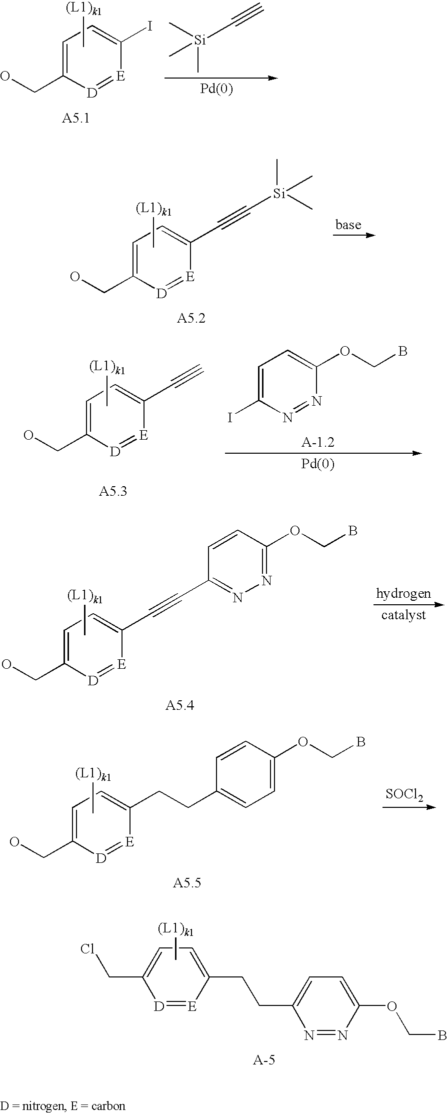

A-5 can be synthesized by Sonogashira reaction of A5.1. with a protected acetylene derivative leading to A5.2, followed by deprotection using a base such as sodium hydroxide resulting in the formation of A5.3. Subsequent Sonogashira reaction of A5.3. with A-1.2 gives A5.4. A5.4 can be reduced catalytically by catalysts such as Raney-Nickel in solvents like DMF under hydrogen atmosphere to A5.5. A-5 is obtained by reacting A5.5 with an chlorinating agent as thionyl chloride.

The synthesis of the precursor A-6 is outlined below.

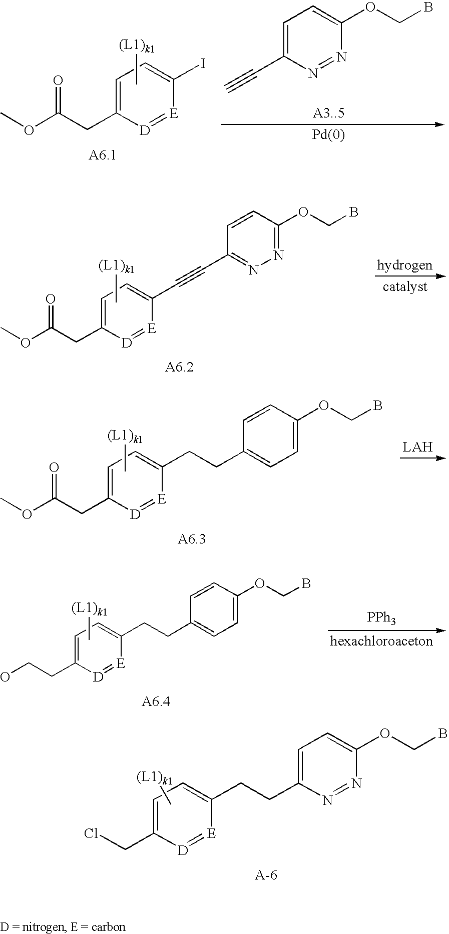

A-6 can be synthesized by Sonogashira reaction of A6.1. with A3.5 giving access to A6.2. A6.2 can be reduced catalytically by catalysts such as Raney-Nickel in solvents like DMF under hydrogen atmosphere to A6.3. Reduction of A6.3 can be achieved by lithiumaluminiumhydride in solvents like THF. Conversion to A-6 can be realized by reaction of A6.4 with hexachloroacetone/triphenylphosphine.

Compounds of the general formula IIIb can be prepared depending on the nature of the linker group X and the groups D and E by the synthesis outlined below:

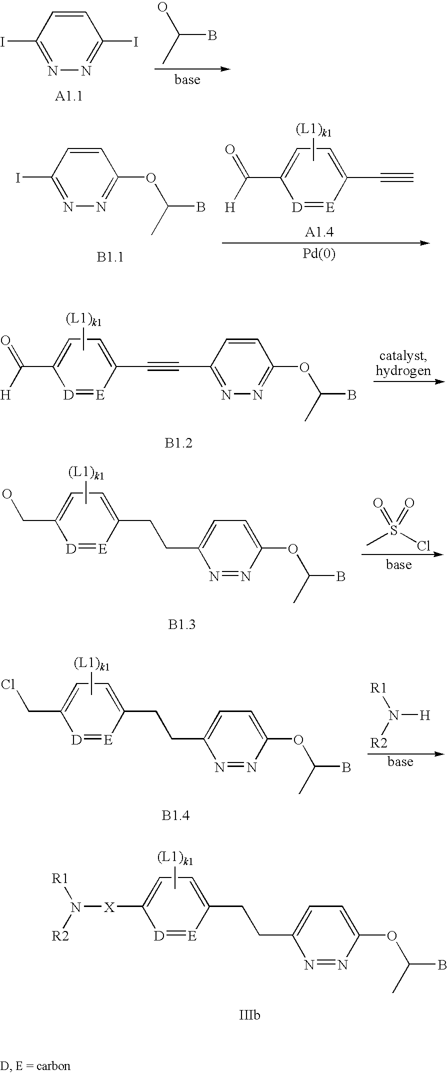

The pyridazine derivative B1.1 is obtained by reaction of the sodium salt of an appropriate alcohol with A1.1 in solvents such as THF at temperatures between 0° C. and 120° C. B1.2 can be obtained by Sonogashira reaction of B1.1 and A1.4 in a solvent such as THF at temperatures between 0° C. and 120° C. B1.2 can be reduced catalytically by catalysts such as Raney-Nickel in solvents like DMF under hydrogen atmosphere to B1.3. The alcoholic function of B1.3 can be transferred into a leaving group such as chloride by reaction with methane sulfonyl chloride in solvents such as methylene chloride in the presence of a base such as triethylamine at temperatures between 0° C. and 120° C. in order to give B1.4. IIIb is obtained by reaction of B1.4 with an appropriate amine in solvents such as THF at temperatures between room temperature and 120° C.

Compounds of the general formula IIIc can be prepared depending on the nature of the linker group X and the groups D and E by the synthesis outlined below:

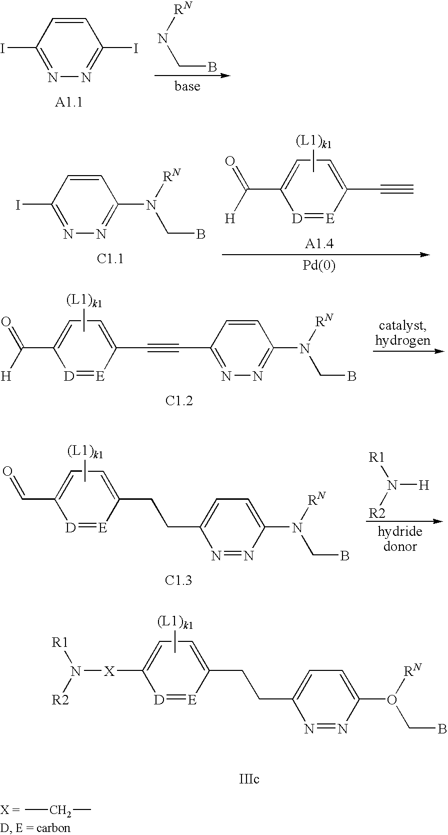

The pyridazine derivative C1.1 is obtained by reaction of the sodium salt of an appropriate amine with A1.1 in solvents such as DMF at temperatures between 0° C. and 140° C. The compound C1.2 can be obtained by Sonogashira reaction of C1.1 and A1.4 in a solvent such as THF at temperatures between 0° C. and 120° C. The compound C1.2 can be reduced catalytically by catalysts such as Raney-Nickel in solvents like DMF under hydrogen atmosphere to C1.3. IIIc is obtained by reductive amination of C.1.3 with hydride donors such as triacetoxyborohydride (either free or resin bound), the appropriate amines and acid like acetic acid in solvents like THF preferably at room temperature.

Compounds of the general formula IIIe can be prepared by the synthesis outlined below:

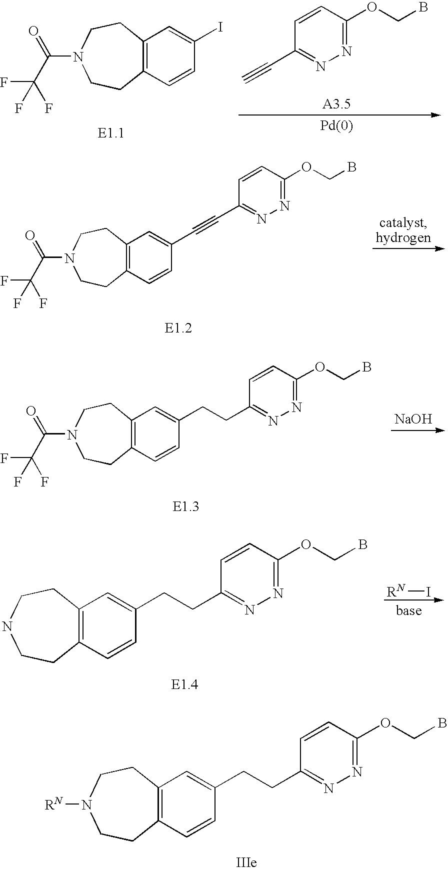

The compound E1.2 can be obtained by Sonogashira reaction of E1.1 and A3.5 in a solvent such as THF at temperatures between 0° C. and 120° C. E1.2 can be reduced catalytically by catalysts such as Raney-Nickel in solvents like DMF under hydrogen atmosphere to E1.3. Deprotection of E1.3 is achieved via cleavage of the trifluoro acetyl group with sodium hydroxide solution in a solvent such as methanol at temperatures between 0° C. and 140° C., preferably at room temperature to give E1.4. The compound E1.4 is alkylated by reaction with an appropriate alkyl halide, preferably an alkyl iodide in the presence of a base such as potassium carbonate in a solvent such as acetone at temperatures between 0° C. and 120° C. to give IIIe.

Compounds of the general formula IIIf can be prepared by the synthesis outlined below:

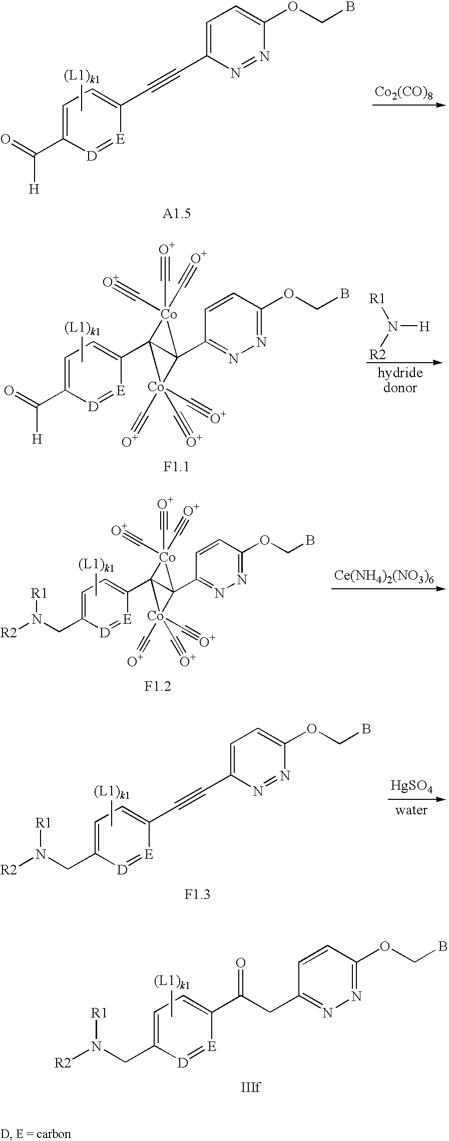

The cobalt complex F1.1 is obtained by reaction of A1.5 with Co₂(O)₈ in a solvent such as toluene at temperatures between 0° C. and 120° C., preferably at room temperature. The compound F1.2 is obtained by reductive amination of F.1.1 with hydride donors such as triacetoxyborohydride (either free or resin bound), the appropriate amines and acid like acetic acid in solvents like THF preferably at room temperature. Reaction of F1.2 with Ce(NH₄)₂(NO₃)₈ in methanol gives F1.3. The compound IIIe can be prepared by reaction of F1.3 with mercury sulphate, water and trifluoro acetic acid in a solvent such as methylene chloride at temperatures between room temperature and 80° C.

Compounds of the general formula IIIk can be prepared by the synthesis outlined below:

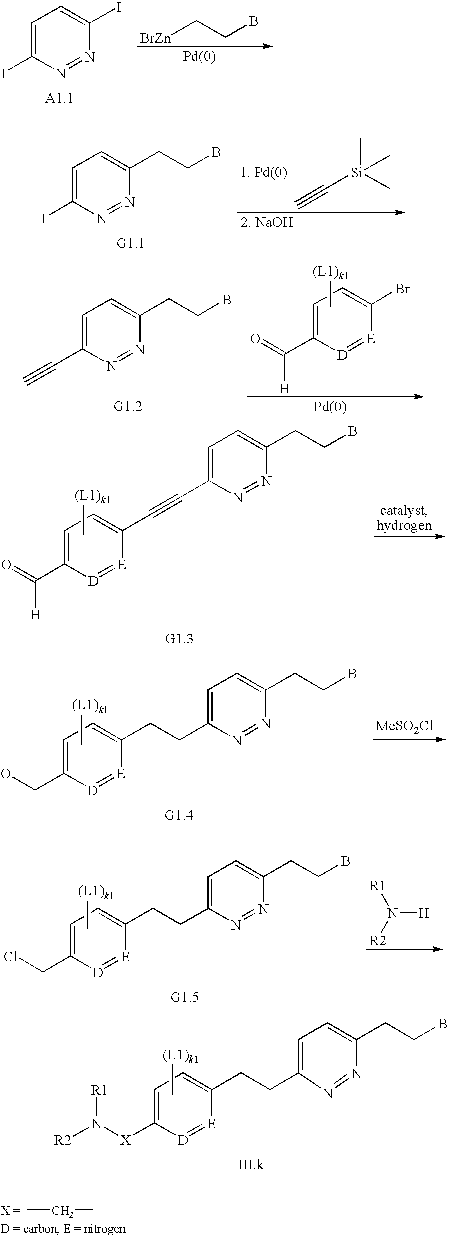

G1.1 can be obtained by reaction of A1.1 with an appropriate zinc reagent in the presence of a catalyst such as palladium. The compound G1.2 can be obtained by Sonogashira reaction of G1.1 with a protected acetylene derivative followed by deprotection. Subsequent Sonogashira reaction gives G1.3. Hydrogenation with a catalyst such as Raney Nickel results in the formation of G1.4. Synthesis of G1.5 is achieved by reaction of G1.4 with methane sulfonyl chloride. Reaction of G1.5 with amines gives derivatives of the type III.k

Compounds of the general formula IIIm can be prepared depending on the nature of the linker group X and the groups D and E by the following methods:
1. Reductive amination of the precursor H-3 with hydride donors such as triacetoxyborohydride (either free or resin bound), the appropriate amines and acid like acetic acid in solvents like THF, preferably at room temperature.
2. Reaction of the precursor H-1, H-2 or H-4 with an appropriate amine and a base such as potassium carbonate in solvents such as acetone at temperatures between room temperature and 120° C.

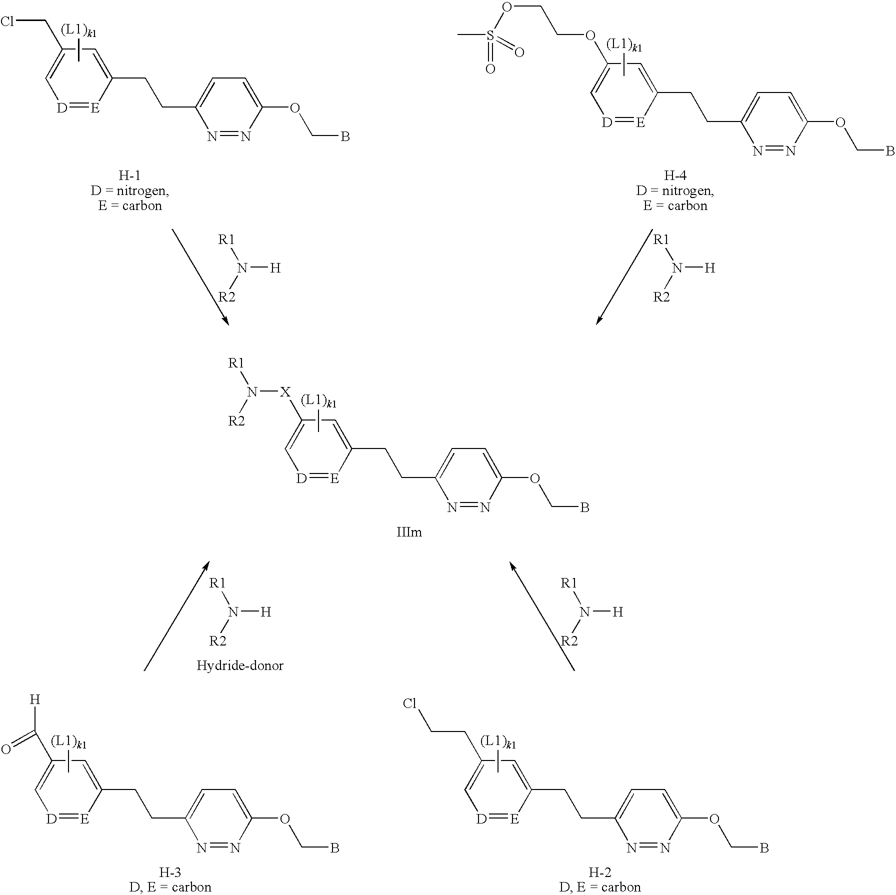

Synthesis of the precursor H-1 can be performed in analogy to the synthesis of precursor A-5.

Synthesis of the precursor H-2 can be performed in analogy to the synthesis of precursor A-6.

Synthesis of the precursor H-3 can be performed in analogy to the synthesis of precursor A-1.

Synthesis of the precursor H-4 can be performed in analogy to the synthesis of precursor A-2.

Stereoisomeric compounds of formula (I) may chiefly be separated by conventional methods. The diastereomers are separated on the basis of their different physico-chemical properties, e.g. by fractional crystallisation from suitable solvents, by high pressure liquid or column chromatography, using chiral or preferably non-chiral stationary phases.

Racemates covered by general formula (I) may be separated for example by HPLC on suitable chiral stationary phases (e.g. Chiral AGP, Chiralpak AD). Racemates which contain a basic or acidic function can also be separated via the diastereomeric, optically active salts which are produced on reacting with an optically active acid, for example (+) or (−)-tartaric acid, (+) or (−)-diacetyl tartaric acid, (+) or (−)-monomethyl tartrate or (+)-camphorsulphonic acid, or an optically active base, for example with (R)-(+)-1-phenylethylamine, (S)-(−)-1-phenylethylamine or (S)-brucine.

According to a conventional method of separating isomers, the racemate of a compound of formula (I) is reacted with one of the above-mentioned optically active acids or bases in equimolar amounts in a solvent and the resulting crystalline, diastereomeric, optically active salts thereof are separated using their different solubilities. This reaction may be carried out in any type of solvent provided that it is sufficiently different in terms of the solubility of the salts. Preferably, methanol, ethanol or mixtures thereof, for example in a ratio by volume of 50:50, are used. Then each of the optically active salts is dissolved in water, carefully neutralised with a base such as sodium carbonate or potassium carbonate, or with a suitable acid, e.g. with dilute hydrochloric acid or aqueous methanesulphonic acid and in this way the corresponding free compound is obtained in the (+) or (−) form.

The (R) or (S) enantiomer alone or a mixture of two optically active diastereomeric compounds of general formula (I) may also be obtained by performing the syntheses described above with a suitable reaction component in the (R) or (S) configuration.

As already mentioned, the compounds of formula (I) may be converted into the salts thereof, particularly for pharmaceutical use into the physiologically and pharmacologically acceptable salts thereof. These salts may be present on the one hand as physiologically and pharmacologically acceptable acid addition salts of the compounds of formula (I) with inorganic or organic acids. On the other hand, in the case of acidically bound hydrogen, the compound of formula (I) may also be converted by reaction with inorganic bases into physiologically and pharmacologically acceptable salts with alkali or alkaline earth metal cations as counter-ion. The acid addition salts may be prepared, for example, using hydrochloric acid, hydrobromic acid, sulphuric acid, phosphoric acid, methanesulphonic acid, ethanesulphonic acid, toluenesulphonic acid, benzenesulphonic acid, acetic acid, fumaric acid, succinic acid, lactic acid, citric acid, tartaric acid or maleic acid. Moreover, mixtures of the above mentioned acids may be used. To prepare the alkali and alkaline earth metal salts of the compound of formula (I) with acidically bound hydrogen the alkali and alkaline earth metal hydroxides and hydrides are preferably used, while the hydroxides and hydrides of the alkali metals, particularly of sodium and potassium, are preferred and sodium and potassium hydroxide are most preferred.

The compounds according to the present invention, including the physiologically acceptable salts, are effective as antagonists of the MCH receptor, particularly the MCH-1 receptor, and exhibit good affinity in MCH receptor binding studies. Pharmacological test systems for MCH-antagonistic properties are described in the following experimental section.

As antagonists of the MCH receptor the compounds according to the invention are advantageously suitable as pharmaceutical active substances for the prevention and/or treatment of symptoms and/or diseases caused by MCH or causally connected with MCH in some other way. Generally the compounds according to the invention have low toxicity, they are well absorbed by oral route and have good intracerebral transitivity, particularly brain accessibility.

Therefore, MCH antagonists which contain at least one compound according to the invention are particularly suitable in mammals, such as for example rats, mice, guinea pigs, hares, dogs, cats, sheep, horses, pigs, cattle, monkeys and humans, for the treatment and/or prevention of symptoms and/or diseases which are caused by MCH or are otherwise causally connected with MCH.

Diseases caused by MCH or otherwise causally connected with MCH are particularly metabolic disorders, such as for example obesity, and eating disorders, such as for example bulimia, including bulimia nervosa. The indication obesity includes in particular exogenic obesity, hyperinsulinaemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity. This range of indications also includes cachexia, anorexia and hyperphagia.

Compounds according to the invention may be particularly suitable for reducing hunger, curbing appetite, controlling eating behaviour and/or inducing a feeling of satiation.

In addition, the diseases caused by MCH or otherwise causally connected with MCH also include hyperlipidaemia, cellulitis, fatty accumulation, malignant mastocytosis, systemic mastocytosis, emotional disorders, affectivity disorders, depression, anxiety states, reproductive disorders, sexual disorders, memory disorders, epilepsy, forms of dementia and hormonal disorders.

Compounds according to the invention are also suitable as active substances for the prevention and/or treatment of other illnesses and/or disorders, particularly those which accompany obesity, such as for example diabetes, diabetes mellitus, particularly type II diabetes, hyperglycaemia, particularly chronic hyperglycaemia, complications of diabetes including diabetic retinopathy, diabetic neuropathy, diabetic nephropathy, etc., insulin resistance, pathological glucose tolerance, encephalorrhagia, cardiac insufficiency, cardiovascular diseases, particularly arteriosclerosis and high blood pressure, arthritis and gonitis.

MCH antagonists and formulations according to the invention may advantageously be used in combination with a dietary therapy, such as for example a dietary diabetes treatment, and exercise.

Another range of indications for which the compounds according to the invention are advantageously suitable is the prevention and/or treatment of micturition disorders, such as for example urinary incontinence, hyperactive bladder, urgency, nycturia, enuresis, while the hyperactive bladder and urgency may or may not be connected with benign prostatic hyperplasia.

Generally speaking, the compounds according to the invention are potentially suitable for preventing and/or treating dependencies, such as for example alcohol and/or nicotine dependency, and/or withdrawal symptoms, such as for example weight gain in smokers coming off nicotine. By “dependency” is generally meant here an irresistible urge to take an addictive substance and/or to perform certain actions, particularly in order to either achieve a feeling of wellbeing or to eliminate negative emotions. In particular, the term “dependency” is used here to denote a dependency on an addictive substance. By “withdrawal symptoms” are meant here, in general, symptoms which occur or may occur when addictive substances are withdrawn from patients dependent on one or more such substances. The compounds according to the invention are potentially suitable particularly as active substances for reducing or ending tobacco consumption, for the treatment or prevention of a nicotine dependency and/or for the treatment or prevention of nicotine withdrawal symptoms, for reducing the craving for tobacco and/or nicotine and generally as an anti-smoking agent. The compounds according to the invention may also be useful for preventing or at least reducing the weight gain typically seen when smokers are coming off nicotine. The substances may also be suitable as active substances which prevent or at least reduce the craving for and/or relapse into a dependency on addictive substances. The term addictive substances refers particularly but not exclusively to substances with a psycho-motor activity, such as narcotics or drugs, particularly alcohol, nicotine, cocaine, amphetamine, opiates, benzodiazepines and barbiturates.

The dosage required to achieve such an effect is conveniently, by intravenous or sub-cutaneous route, 0.001 to 30 mg/kg of body weight, preferably 0.01 to 5 mg/kg of body weight, and by oral or nasal route or by inhalation, 0.01 to 50 mg/kg of body weight, preferably 0.1 to 30 mg/kg of body weight, in each case 1 to 3× daily.

For this purpose, the compounds prepared according to the invention may be formulated, optionally in conjunction with other active substances as described hereinafter, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, lozenges, powders, granules, solutions, emulsions, syrups, aerosols for inhalation, ointments or suppositories.

In addition to pharmaceutical compositions the invention also includes compositions containing at least one alkyne compound according to the invention and/or a salt according to the invention optionally together with one or more physiologically acceptable excipients. Such compositions may also be for example foodstuffs which may be solid or liquid, in which the compound according to the invention is incorporated.

For the above mentioned combinations it is possible to use as additional active substances particularly those which for example potentiate the therapeutic effect of an MCH antagonist according to the invention in terms of one of the indications mentioned above and/or which make it possible to reduce the dosage of an MCH antagonist according to the invention. Preferably one or more additional active substances are selected from among
active substances for the treatment of diabetes,
active substances for the treatment of diabetic complications,
active substances for the treatment of obesity, preferably other than MCH antagonists,
active substances for the treatment of high blood pressure,
active substances for the treatment of hyperlipidaemia, including arteriosclerosis,
active substances for the treatment of dyslipidaemia, including arteriosclerosis,
active substances for the treatment of arthritis,
active substances for the treatment of anxiety states,
active substances for the treatment of depression.

The above mentioned categories of active substances will now be explained in more detail by means of examples.

Examples of active substances for the treatment of diabetes are insulin sensitisers, insulin secretion accelerators, biguanides, insulins, α-glucosidase inhibitors, β3 adreno-receptor agonists.
Insulin sensitisers include glitazones, particularly pioglitazone and its salts (preferably hydrochloride), troglitazone, rosiglitazone and its salts (preferably maleate), JTT-501, GI-262570, MCC-555, YM-440, DRF-2593, BM-13-1258, KRP-297, R-119702 and GW-1929.
Insulin secretion accelerators include sulphonylureas, such as for example tolbutamide, chloropropamide, tolazamide, acetohexamide, glyclopyramide and its ammonium salts, glibenclamide, gliclazide, glimepiride. Further examples of insulin secretion accelerators are repaglinide, nateglinide, mitiglinide (KAD-1229) and JTT-608.
Biguanides include metformin, buformin and phenformin.
Insulins include those obtained from animals, particularly cattle or pigs, semisynthetic human insulins which are synthesised enzymatically from insulin obtained from animals, human insulin obtained by genetic engineering, e.g. from Escherichi coli or yeasts. Moreover, the term insulin also includes insulin-zinc (containing 0.45 to 0.9 percent by weight of zinc) and protamine-insulin-zinc obtainable from zinc chloride, protamine sulphate and insulin. Insulin may also be obtained from insulin fragments or derivatives (for example INS-1, etc.).
Insulin may also include different kinds, e.g. with regard to the onset time and duration of effect (“ultra immediate action type”, “immediate action type”, “two phase type”, “intermediate type”, “prolonged action type”, etc.), which are selected depending on the pathological condition of the patient.
α-Glucosidase inhibitors include acarbose, voglibose, miglitol, emiglitate.
β₃ Adreno receptor agonists include AJ-9677, BMS-196085, SB-226552, AZ40140.
Active substances for the treatment of diabetes other than those mentioned above include ergoset, pramlintide, leptin, BAY-27-9955 as well as glycogen phosphorylase inhibitors, sorbitol dehydrogenase inhibitors, protein tyrosine phosphatase 1B inhibitors, dipeptidyl protease inhibitors, glipazide, glyburide.

Active substances for the treatment of diabetes or diabetic complications furthermore include for example aldose reductase inhibitors, glycation inhibitors and protein kinase C inhibitors, DPPIV blockers, GLP-1 or GLP-2 analogues and SGLT-2 inhibitors.
Aldose reductase inhibitors are for example tolrestat, epalrestat, imirestat, zenarestat, SNK-860, zopolrestat, ARI-50i, AS-3201.
An example of a glycation inhibitor is pimagedine.
Protein Kinase C inhibitors are for example NGF, LY-333531.
DPPIV blockers are for example LAF237 (Novartis), MK431 (Merck) as well as 815541, 823093 and 825964 (all GlaxoSmithkline).
GLP-1 analogues are for example Liraglutide (NN2211) (NovoNordisk), CJC1131 (Conjuchem), Exenatide (Amylin).
SGLT-2 inhibitors are for example AVE-2268 (Aventis) and T-1095 (Tanabe, Johnson&Johnson).
Active substances other than those mentioned above for the treatment of diabetic complications include alprostadil, thiapride hydrochloride, cilostazol, mexiletine hydrochloride, ethyl eicosapentate, memantine, pimagedine (ALT-711).

Active substances for the treatment of obesity, preferably other than MCH antagonists, include lipase inhibitors and anorectics.
A preferred example of a lipase inhibitor is orlistat.
Examples of preferred anorectics are phentermine, mazindol, dexfenfluramine, fluoxetine, sibutramine, baiamine, (S)-sibutramine, SR-141716, NGD-95-1.
Active substances other than those mentioned above for the treatment of obesity include lipstatin.
Moreover, for the purposes of this application, the active substance group of anti-obesity active substances also includes the anorectics, of which the β₃ agonists, thyromimetic active substances and NPY antagonists should be emphasised. The range of substances which may be considered as preferred anti-obesity or anorectic active substances is indicated by the following additional list, by way of example: phenylpropanolamine, ephedrine, pseudoephedrine, phentermine, a cholecystokinin-A (hereinafter referred to as CCK-A) agonist, a monoamine reuptake inhibitor (such as for example sibutramine), a sympathomimetic active substance, a serotonergic active substance (such as for example dexfenfluramine, fenfluramine, a 5-HT2C agonist such as BVT.933 or APD356, or duloxetine), a dopamine antagonist (such as for example bromocriptine or pramipexol), a melanocyte-stimulating hormone receptor agonist or mimetic, an analogue of melanocyte-stimulating hormone, a cannabinoid receptor antagonist (Rimonabant, ACOMPLIA™), an MCH antagonist, the OB protein (hereinafter referred to as leptin), a leptin analogue, a fatty acid synthase (FAS) antagonist, a leptin receptor agonist, a galanine antagonist, a GI lipase inhibitor or reducer (such as for example orlistat). Other anorectics include bombesin agonists, dehydroepiandrosterone or its analogues, glucocorticoid receptor agonists and antagonists, orexin receptor antagonists, urocortin binding protein antagonists, agonists of the Glucagon-like Peptide-1 receptor, such as for example exendin, AC 2993, CJC-1131, ZP10 or GRT0203Y, DPPIV inhibitors and ciliary neurotrophic factors, such as for example axokines. In this context mention should also be made of the forms of therapy which produce weight loss by increasing the fatty acid oxidation in the peripheral tissue, such as for example inhibitors of acetyl-CoA carboxylase.

Active substances for the treatment of high blood pressure include inhibitors of angiotensin converting enzyme, calcium antagonists, potassium channel openers and angiotensin II antagonists.
Inhibitors of angiotensin converting enzyme include captopril, enalapril, alacepril, delapril (hydrochloride), lisinopril, imidapril, benazepril, cilazapril, temocapril, trandolapril, manidipine (hydrochloride).
Examples of calcium antagonists are nifedipine, amlodipine, efonidipine, nicardipine.
Potassium channel openers include levcromakalim, L-27152, AL0671, NIP-121.
Angiotensin II antagonists include telmisartan, losartan, candesartan cilexetil, valsartan, irbesartan, CS-866, E4177.

Active substances for the treatment of hyperlipidaemia, including arteriosclerosis, include HMG-CoA reductase inhibitors, fibrate compounds.
HMG-CoA reductase inhibitors include pravastatin, simvastatin, lovastatin, atorvastatin, fluvastatin, lipantil, itavastatin, ZD-4522 and their salts.
Fibrate compounds include fenofibrate, bezafibrate, clinofibrate, clofibrate and simfibrate.

Active substances for the treatment of dyslipidaemia, including arteriosclerosis, include e.g. medicaments which raise the HDL level, such as e.g. nicotinic acid and derivatives and preparations thereof, such as e.g. niaspan, as well as agonists of the nicotinic acid receptor.

Active substances for the treatment of arthritis include NSAIDs (non-steroidal antiinflammatory drugs), particularly COX2 inhibitors, such as for example meloxicam or ibuprofen.

Active substances for the treatment of anxiety states include chlordiazepoxide, diazepam, oxozolam, medazepam, cloxazolam, bromazepam, lorazepam, alprazolam, fludiazepam.

Active substances for the treatment of depression include fluoxetine, fluvoxamine, imipramine, paroxetine, sertraline.

The dosage for these active substances is conveniently 1/5 of the lowest normal recommended dose up to 1/1 of the normal recommended dose.

In another embodiment the invention also relates to the use of at least one alkyne compound according to the invention and/or a salt according to the invention for influencing the eating behaviour of a mammal. This use is particularly based on the fact that compounds according to the invention may be suitable for reducing hunger, curbing appetite, controlling eating behaviour and/or inducing a feeling of satiety. The eating behaviour is advantageously influenced so as to reduce food intake. Therefore, the compounds according to the invention are advantageously used for reducing body weight. Another use according to the invention is the prevention of increases in body weight, for example in people who had previously taken steps to lose weight and are interested in maintaining their lower body weight. A further use may be the prevention of weight gain in a ω-medication with a substance generally causing weight gain (such a glitazones). According to this embodiment it is preferably a non-therapeutic use. Such a non-therapeutic use might be a cosmetic use, for example to alter the external appearance, or an application to improve general health. The compounds according to the invention are preferably used non-therapeutically for mammals, particularly humans, not suffering from any diagnosed eating disorders, no diagnosed obesity, bulimia, diabetes and/or no diagnosed micturition disorders, particularly urinary incontinence. Preferably, the compounds according to the invention are suitable for non-therapeutic use in people whose BMI (body mass index), defined as their body weight in kilograms divided by their height (in metres) squared, is below a level of 30, particularly below 25.

The Examples that follow are intended to illustrate the invention:

### Preliminary Remarks:

As a rule, ¹H-NMR and/or mass spectra have been obtained for the compounds prepared.

The R_{f} values are determined using ready-made silica gel 60 TLC plates F₂₅₄ (E. Merck, Darmstadt, Item no. 1.05714) without chamber saturation or using ready-made aluminium oxide 60 F₂₅₄ TLC plates (E. Merck, Darmstadt, Item no. 1.05713) without chamber saturation. The ratios given for the eluents relate to units by volume of the solvent in question. The units by volume for NH₃ relate to a concentrated solution of NH₃ in water. Silica gel made by Millipore (MATREX™, 35-70 my) is used for chromatographic purification. Alox (E. Merck, Darmstadt, aluminium oxide 90 standardised, 63-200 μm, Item no. 1.01097.9050) is used for chromatographic purification.

The HPLC data given are measured under the following parameters:

mobile phase A: water:formic acid 99.9:0.1
mobile phase B: acetonitrile:formic acid 99.9:0.1
method A: analytical column: X-terra™ MS C18; 2.5 μm, 4.6 mm×30 mm; column temperature: 25° C.
   gradient:

| time in | | | flow rate |
|---|---|---|---|
| min | % A | % B | in ml/min |
| 0.00 | 95.0 | 5.0 | 1.00 |
| 0.10 | 95.0 | 5.0 | 1.00 |
| 3.10 | 2.00 | 98.00 | 1.00 |
| 4.50 | 2.00 | 98.00 | 1.00 |
| 5.00 | 95.0 | 5.0 | 1.00 |

method B: analytical column: Zorbax column (Agilent Technologies), SB (Stable Bond)—C18; 3.5 μm; 4.6 mm×75 mm; column temperature: 30° C.
   gradient:

| time in | | | flow rate |
|---|---|---|---|
| min | % A | % B | in ml/min |
| 0.00 | 95.0 | 5.0 | 1.60 |
| 4.50 | 10.0 | 90.0 | 1.60 |
| 5.00 | 10.0 | 90.0 | 1.60 |
| 5.50 | 95.0 | 5.0 | 1.60 |

method C: analytical column: Zorbax column (Agilent Technologies), SB (Stable Bond)—C18; 3.5 μm; 4.6 mm×75 mm; column temperature: 30° C.
   gradient:

| time in min | % A | % B | flow rate in ml/min |
|---|---|---|---|
| 0.00 | 95.0 | 5.0 | 0.80 |
| 9.00 | 10.0 | 90.0 | 0.80 |
| 11.0 | 90.0 | 10.00 | 0.80 |

HPLC separations on a preparative scale are done under the following parameters:

mobile phase A: water:trifluoroacetic acid 99.8:0.2
mobile phase B: acetonitrile:100
method 1 (Method amslpolar3): preparative column: Atlantis™ column (Waters technologies) DC18 OBD™ 5 μm 30×100 mm column temperature: 25° C.
   gradient:

| time in min | % A | % B | flow rate in ml/min |
|---|---|---|---|
| 0.00 | 95.0 | 5.0 | 63.00 |
| 2.00 | 95.0 | 5.0 | 63.00 |
| 2.50 | 95.0 | 5..0 | 63.00 |
| 9.50 | 60.0 | 40.0 | 63.00 |
| 10.00 | 5.00 | 95.0 | 63.00 |
| 12.00 | 5.00 | 95.0 | 63.00 |
| 12.50 | 90.0 | 10.0 | 63.00 |
| 14.50 | 90.0 | 10.0 | 63.00 |
| 15.00 | 90.0 | 10.0 | 0 |

Method 2 (Method amslpolar2): preparative column: Xterra™ column (Waters technologies) MSC18 Xterra™ ODB™ 5 μm 30×100 mm column temperature 25° C.
   gradient:

| time in min | % A | % B | flow rate in ml/min |
|---|---|---|---|
| 0.00 | 95.0 | 5.0 | 63.00 |
| 2.00 | 95.0 | 5.0 | 63.00 |
| 2.50 | 95.0 | 5..0 | 63.00 |
| 9.50 | 60.0 | 40.0 | 63.00 |
| 10.00 | 5.00 | 95.0 | 63.00 |
| 12.00 | 5.00 | 95.0 | 63.00 |
| 12.50 | 90.0 | 10.0 | 63.00 |
| 14.50 | 90.0 | 10.0 | 63.00 |
| 15.00 | 90.0 | 10.0 | 0 |

Method 3 (Method amslpolar1): preparative column: Xterra™ column (Waters technologies) MSC18 xterra ODB™ 5 μm 30×100 mm column temperature 25° C.
   gradient:

| time in min | % A | % B | flow rate in ml/min |
|---|---|---|---|
| 0.00 | 90.0 | 10.0 | 63.00 |
| 2.00 | 90.0 | 10.0 | 63.00 |
| 2.50 | 90.0 | 10.0 | 63.00 |
| 9.50 | 46.0 | 54.0 | 63.00 |
| 10.00 | 5.00 | 95.0 | 63.00 |
| 12.00 | 5.00 | 95.0 | 63.00 |
| 12.50 | 90.0 | 10.0 | 63.00 |
| 14.50 | 90.0 | 10.0 | 63.00 |
| 15.00 | 90.0 | 10.0 | 0 |

Method 4 (Method amslstandard): preparative column: Xterra™ column (Waters technologies) MSC18 xterra ODB™ 5 μm 30×100 mm column temperature 25° C.
   gradient:

| time in min | % A | % B | flow rate in ml/min |
|---|---|---|---|
| 0.00 | 90.0 | 10.0 | 63.00 |
| 2.00 | 90.0 | 10.0 | 63.00 |
| 2.50 | 67.0 | 33.0 | 63.00 |
| 9.50 | 33.0 | 67.0 | 63.00 |
| 10.00 | 5.00 | 95.0 | 63.00 |
| 12.00 | 5.00 | 95.0 | 63.00 |
| 12.50 | 90.0 | 10.0 | 63.00 |
| 14.50 | 90.0 | 10.0 | 63.00 |
| 15.00 | 90.0 | 10.0 | 0 |

Method 5 (Method amslunpolar1): preparative column: Xterra™ column (Waters technologies) MSC18 xterra ODB™ 5 μm 30×100 mm column temperature 25° C.
   gradient:

| time in min | % A | % B | flow rate in ml/min |
|---|---|---|---|
| 0.00 | 90.0 | 10.0 | 63.00 |
| 2.00 | 90.0 | 10.0 | 63.00 |
| 2.50 | 53.0 | 47.0 | 63.00 |
| 9.50 | 18.0 | 82.0 | 63.00 |
| 10.00 | 5.00 | 95.0 | 63.00 |
| 12.00 | 5.00 | 95.0 | 63.00 |
| 12.50 | 90.0 | 10.0 | 63.00 |
| 14.50 | 90.0 | 10.0 | 63.00 |
| 15.00 | 90.0 | 10.0 | 0 |

Method 6 (Method amslunpolar2): preparative column: Xterra™ column (Waters technologies) MSC18 xterra ODB™ 5 μm 30×100 mm column temperature 25° C.
   gradient:

| time in min | % A | % B | flow rate in ml/min |
|---|---|---|---|
| 0.00 | 90.0 | 10.0 | 63.00 |
| 2.00 | 90.0 | 10.0 | 63.00 |
| 2.50 | 39.0 | 61.0 | 63.00 |
| 9.50 | 4.0 | 96.0 | 63.00 |
| 10.00 | 5.00 | 95.0 | 63.00 |
| 12.00 | 5.00 | 95.0 | 63.00 |
| 12.50 | 90.0 | 10.0 | 63.00 |
| 14.50 | 90.0 | 10.0 | 63.00 |
| 15.00 | 90.0 | 10.0 | 0 |

The following abbreviations are used above and hereinafter:
abs. absolute
Cbz benzyloxycarbonyl
conc. concentrated
DMF N,N-dimethylformamide
EII electron impact ionisation
ether diethyl ether
EtOAc ethyl acetate
EtOH ethanol
Fmoc 9-fluorenylmethoxycarbonyl
HCl hydrochloric acid
MeOH methanol
Ph phenyl
RT room or ambient temperature (about 20° C.)
TBTU 2-(1H-benzotriazol-1-yl)-1,1,3,3-tetramethyluronium-tetrafluoroborate
THF tetrahydrofuran

### Preparation of the Starting Compounds

### Example I.1

### 4-[2-(6-Benzyloxy-pyridazin-3-yl)-ethyl]-benzaldehyde

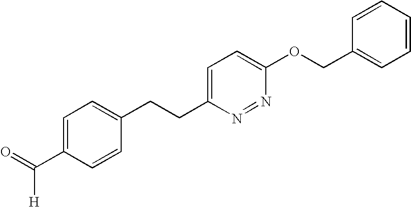

### I.1.a

### 3,6-Diiodopyridazine

A mixture 10.0 g (67.1 mmol) of 3,6-dichloropyridazine and 40 ml of hydroiodic acid (57% in water) is stirred for 24 hours at 70° C. After cooling down the reaction mixture is poured on ice, neutralized with 120 ml of potassium hydroxide solution (20% in water) and filtered. The residue is washed with 1000 ml of water, 50 ml of sodium thiosulfate solution (10% in water) and 10 ml of n-hexane. The residue is recrystallised from EtOAc.

Yield: 9.4 g (42% of theory),

### I.1.b

### 3-Benzyloxy-6-iodo-pyridazine

A mixture 9.956 g (30 mmol) of 3,6-diiodopyridazine and 3.904 ml sodium benzyloxide (1M in benzylic alcohol) in 300 ml of toluene is stirred for 16 hours at 60° C. The solvent is evaporated. The residue is extracted with water and EtOAc. The organic layer is dried with sodium sulphate.

Yield: 7.2 g (77% of theory),

retention time (HPLC): 4.394 min (method B)

C₁₁H₉IN₂O

EII Mass spectrum: m/z=313/314 [M+H]⁺

### I.1.c

### 4-(6-Benzyloxy-pyridazin-3-ylethynyl)-benzaldehyde

A mixture of 2.3 g (7.06 mmol) of cesium carbonate and 0.9 g (2.88 mmol) of 3-benzyloxy-6-iodo-pyridazine in 30 ml of dry THF is cooled with a mixture of solid carbon dioxide and methanol. The mixture is degassed and flushed with argon. Then 115 mg of (0.164 mmol) bis-(triphenylphosphine)-palladium dichloride and 50 mg (0.263 mmol) copper(I)-iodide are added. The resulting mixture is degassed and flushed with argon. 0.39 g (3 mmol) of 4-ethynyl-benzaldehyde are added and the mixture is stirred for two hours at room temperature. After that time 0.2 g (1.53 mmol) of 4-ethynyl-benzaldehyde are added and the mixture is stirred for one hour. The solvent is evaporated. A mixture of water and EtOAc is added to the residue. The mixture is filtered and the residue is stirred with diisopropylether. The product is isolated by filtration.

Yield: 0.8 g (70% of theory),

R_{f} value: 0.90 (silica gel, methylene chloride/methanol=20:1)

C₂₀H₁₄N₂O₂

EII Mass spectrum: m/z=315 [M+H]⁺

### I.1.d

### 4-[2-(6-Benzyloxy-pyridazin-3-yl)-ethyl]-benzaldehyde

A mixture of 0.2 g (0.63 mmol) of 4-(6-benzyloxy-pyridazin-3-ylethynyl)-benzaldehyde and 50 mg of raney-nickel in 10 ml of dry DMF is stirred for nine hours at room temperature in a hydrogen atmosphere (3-4 bar). The mixture is filtrated, The solvent is evaporated. The residue is purified by silica gel column chromatography with methylene chloride/MeOH/0.1% ammonia as eluent.

Yield: 0.08 g (56% of theory),

C₂₀H₁₈N₂O₂

EII Mass spectrum: m/z=319 [M+H]⁺

The following compounds are synthesised analogously to the method described above:

| | | mass | Retention |
|---|---|---|---|
| Example | Structure | spectrum | time (HPLC) |
| 1.2 | 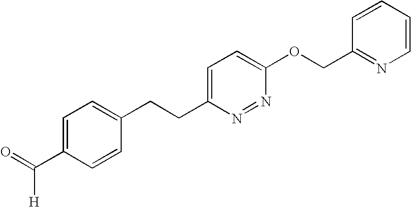 | 320 [M + H]⁺ | |
| 1.3 | 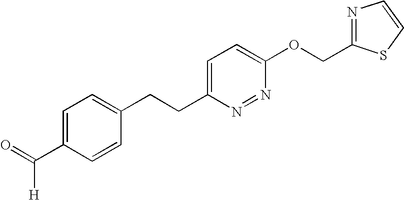 | 326 [M + H]⁺ | 3.35 (method A) |
| 1.4 | 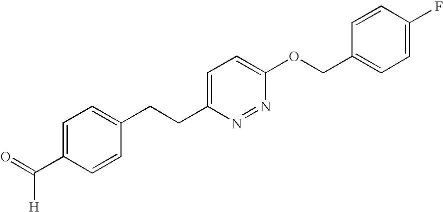 | 337 [M + H]⁺ | 3.17 (method A) |
| 1.5 | 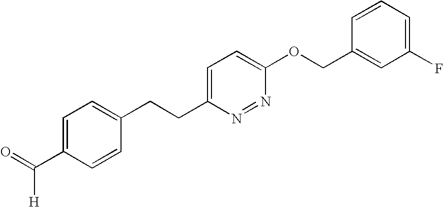 | 337 [M + H]⁺ | |
| 1.6 | 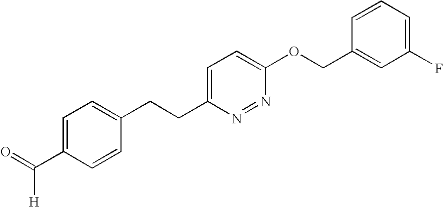 | 337 [M + H]⁺ | |
| 1.7 | 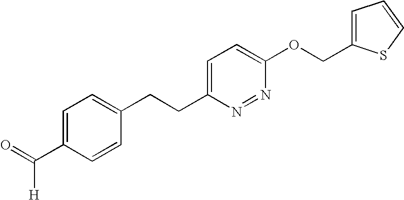 | 325 [M + H]⁺ | |
| 1.8 | 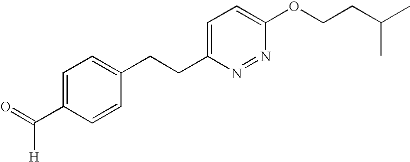 | 299 [M + H]⁺ | |
| 1.9 | 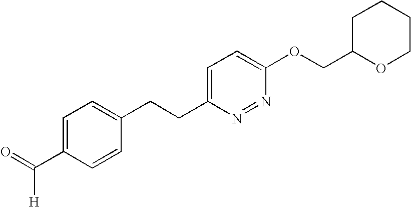 | 327 [M + H]⁺ | |

### Example I.10

### 4-[2-(6-Benzylamino-pyridazin-3-yl)-ethyl]benzaldehyde

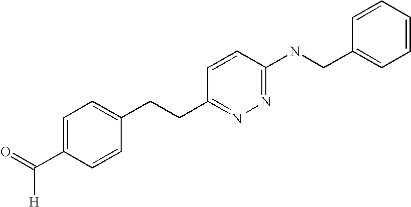

### I.10.a Benzyl-(6-iodo-pyridazin-3-yl)-amine

471 mg (10.8 mmol) sodium hydride (55%) are added at 0° C. under nitrogen atmosphere to a mixture of 1.084 ml (9.8 mmol) benzylamine and 25 ml dry DMF. The reaction mixture is stirred for one hour at room temperature. Then 3.26 g (9.8 mmol) 3,6-diiodo-pyridazine are added and the reaction mixture is stirred at 100° C. for 18 hours. The reaction mixture is concentrated. Methylene chloride is added to the residue and the mixture is extracted with water. The organic phase is dried over sodium sulphate and concentrated. Purification is achieved by silica gel column chromatography with petrol ether/EtOAc as eluent.

Yield: 0.23 g (8% of theory),

R_{f} value: 0.55 (silica gel, petrol ether/EtOAc=1:1)

### I.10.b 4-(6-Benzylamino-pyridazin-3-ylethynyl)-benzaldehyde

Prepared analogously to example 1.1.c from benzyl-(6-iodo-pyridazin-3-yl)-amine and 4-ethynyl-benzaldehyde.

Yield: 0.73 g (94% of theory),

R_{f} value: 0.74 (silica gel, methylene chloride/MeOH/ammonia solution=90:10:1)

C₂₀H₁₅N₃O

EII mass spectrum: m/z=314 [M+H]⁺

### I.10.c 4-[2-(6-Benzylamino-pyridazin-3-yl)-ethyl]benzaldehyde

Prepared analogously to example 1.1.d from 4-(6-benzylamino-pyridazin-3-ylethynyl)-benzaldehyde.

Yield: 0.45 g (76% of theory),

R_{f} value: 0.57 (silica gel, methylene chloride/methanol/ammonia solution=90:10:1)

C₂₀H₁₉N₃O

EII mass spectrum: m/z=318 [M+H]⁺

### Example I.11

### 4-{2-[6-(Benzyl-methyl-amino)-pyridazin-3-yl]-ethyl}-benzaldehyde

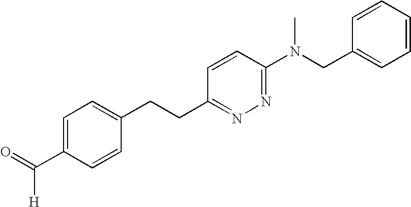

### I.11.a Benzyl-(6-iodo-pyridazin-3-yl)-methyl-amine

Prepared analogously to example I.7.a from 3,6-diiodo-pyridazine and N-methylbenzyl-amine.

Yield: 1 g (42% of theory),

R_{f} value: 0.85 (silica gel, methylene chloride/methanol/ammonia solution=90:10:1

### I.11.b 4-[6-(Benzyl-methyl-amino)-pyridazin-3-ylethynyl]-benzaldehyde

Prepared analogously to example 1.1.c from benzyl-(6-iodo-pyridazin-3-yl)-methyl-amine and 4-ethynyl-benzaldehyde.

Yield: 1 g (98% of theory),

C₂₁H₁₇N₃O

EII mass spectrum: m/z=328 [M+H]⁺

### I.11.c 4-[2-(6-Benzylamino-pyridazin-3-yl)-ethyl]benzaldehyde

Prepared analogously to example 1.1.d from 4-[6-(benzyl-methyl-amino)-pyridazin-3-ylethynyl]-benzaldehyde.

Yield: 0.45 g (76% of theory),

R_{f} value: 0.54 (silica gel, methylene chloride/methanol/ammonia solution=90:10:1)

C₂₁H₂₁N₃O

EII mass spectrum: m/z=332 [M+H]⁺

### Example I.12

### Methanesulfonic acid 2-[2-(6-benzyloxy-pyridazin-3-yl)-ethyl]thiazol-4-ylmethyl ester

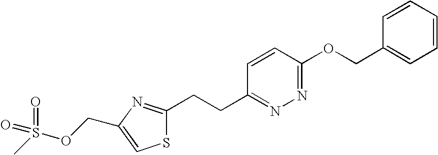

### I.12.a 2-Iodo-thiazole-4-carboxylic acid ethyl ester

4.681 ml (34.84 mmol) isoamylnitrate are added at 0° C. in the dark to a mixture of 4 g (23.22 mmol) ethyl-2-amino-1,3-thiazole-4-carboxylate and 25 ml diiodomethane. The reaction mixture is stirred for 4 days at room temperature in the dark. The reaction mixture is purified by silica gel column chromatography with methylene chloride/methanol as eluent. The crude product is further purified by HPLC (method 3). Product fractions are extracted with methylene chloride. The combined organic phases are dried over sodium sulphate and concentrated.

Yield: 2.2 g (33% of theory),

retention time (HPLC): 2.78 min (method A)

C₆H₆INO₂S

EII mass spectrum: m/z=284 [M+H]⁺

### I.12.b 2-(6-Benzyloxy-pyridazin-3-ylethynyl)-thiazole-4-carboxylic acid ethyl ester

Prepared analogously to example III.1.c from 3-benzyloxy-6-ethynyl-pyridazine (example III.1.b) and 2-iodo-thiazole-4-carboxylic acid ethyl ester.

Yield: 0.4 g (97% of theory),

C₁₉H₁₅N₃O₃S

EII mass spectrum: m/z=366 [M+H]⁺

### I.12.c 2-[2-(6-Benzyloxy-pyridazin-3-yl)-ethyl]thiazole-4-carboxylic acid ethyl ester

Prepared analogously to example III.1.d from 2-(6-benzyloxy-pyridazin-3-ylethynyl)-thiazole-4-carboxylic acid ethyl ester.

Yield: 40 mg (12% of theory),

C₁₉H₁₉N₃O₃S

EII mass spectrum: m/z=370 [M+H]⁺

### I.12.d {2-[2-(6-Benzyloxy-pyridazin-3-yl)-ethyl]-thiazol-4-yl}-methanol

To a solution of 40 mg (0.1 mmol) 2-[2-(6-benzyloxy-pyridazin-3-yl)-ethyl]-thiazole-4-carboxylic acid ethyl ester in 2 ml dry THF is added 0.1 ml lithium aluminium hydride solution (1M in THF). The reaction mixture is stirred for two hours at room temperature. Then water is added slowly and the resulting mixture is extracted with EtOAc. The organic phase is dried over sodium sulphate and concentrated.

Yield: 40 mg (12% of theory),

C₁₇H₁₇N₃O₂S

EII mass spectrum: m/z=328 [M+H]⁺

### I.12.e Methanesulfonic acid 2-[2-(6-benzyloxy-pyridazin-3-yl)-ethyl]-thiazol-4-ylmethyl ester

Prepared analogously to example IV.2.f from {2-[2-(6-benzyloxy-pyridazin-3-yl)-ethyl]-thiazol-4-yl}-methanol

Yield: 30 mg (97% of theory),

retention time (HPLC): 2.92 min (method A)

### Example I.13

### 4-{2-[6-(2-Thiophen-3-yl-ethyl)-pyridazin-3-yl]-ethyl}-benzaldehyde

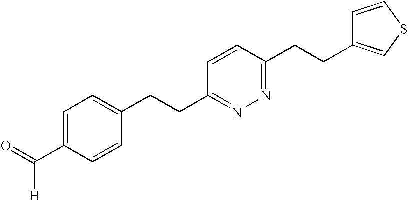

### I.13.a 3-Iodo-6-thiophen-3-ylethynyl-pyridazine

Prepared analogously to example III.1.c from 3,6-diiodo-pyridazine and 3-ethynyl-thiophene.

Yield: 0.4 g (97% of theory),

R_{f} value: 0.5 (silica gel, methylene chloride)

C₁₉H₁₅N₃O₃S

EII mass spectrum: m/z=366 [M+H]⁺

### I.13.b 4-(6-Thiophen-3-ylethynyl-pyridazin-3-ylethynyl)-benzaldehyde

Prepared analogously to example III.1.c from 3-Iodo-6-thiophen-3-ylethynyl-pyridazine and 4-ethynyl-benzaldehyde.

Yield: 0.78 g (80% of theory),

C₁₉H₁₀N₂OS

EII mass spectrum: m/z=315 [M+H]⁺

### I.13.c 4-{2-[6-(2-Thiophen-3-yl-ethyl)-pyridazin-3-yl]-ethyl}-benzaldehyde

Prepared analogously to example III.1.d from 4-(6-Thiophen-3-ylethynyl-pyridazin-3-ylethynyl)-benzaldehyde.

Yield: 0.21 g (27% of theory),

C₁₉H₁₈N₂OS

EII mass spectrum: m/z=323 [M+H]⁺

### Example I.14

### 3-[2-(6-Benzyloxy-pyridazin-3-yl)-ethyl]-benzaldehyde

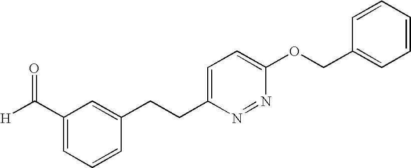

### I.14.a 3-[2-(6-Benzyloxy-pyridazin-3-yl)-ethynyl]benzaldehyde

A solution of 3.12 g (13.45 mmol) 3-iodo-benzaldehyde in 100 ml dry THF is degassed as described in example III1.a. 625 mg (0.89 mmol) bis-(triphenylphosphin)-palladium-1′-chloride and 170 mg (0.89 mmol) copper iodide are added and the reaction mixture is degassed again. Then 3.39 g (16.13 mmol) 3-benzyloxy-6-ethynyl-pyridazine and 5.27 ml (37.84 mmol) triethylamine are added and the mixture is stirred for 2 hours at room temperature. The mixture is poured onto water and extracted with dichloromethane. The organic layer is dried over sodium sulphate and concentrated. The residue is purified by silica gel column chromatography with petrolether/ethyl acetate (1:1) as eluent. The solid is washed with diisopropylether and dried.

Yield: 2.26 g (54% of theory),

C₁₀H₁₄N₂O₂

EII Mass spectrum: m/z=315 [M+H]⁺

### I.14.b 3-[2-(6-Benzyloxy-pyridazin-3-yl)-ethyl]-benzaldehyde

A mixture of 2.26 g (7.19 mmol) 3-[2-(6-benzyloxy-pyridazin-3-ylethynyl)-benzaldehyde and 0.5 g Raney/Ni in 120 ml dry DMF is stirred under hydrogen atmosphere (40-50 psi) at room temperature for 6 hours. When the reduction is completed the reaction mixture is filtered and concentrated. The residue is purified by silica gel column chromatography with petrolether/ethyl acetate (1:1) as eluent.

Yield: 1.22 g (53% of theory),

C₂₀H₁₈N₂O₂

EII Mass spectrum: m/z=319 [M+H]⁺

### Example II.1

### Azetidin-1-yl-piperidin-4-yl-methanone

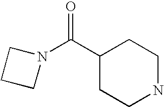

### II.1.a

### 4-(Azetidine-1-carbonyl)-piperidine-1-carboxylic acid tert-butyl ester

1 g (4.362 mmol) piperidine-1,4-dicarboxylic acid mono-tert-butyl ester is dissolved in 10 ml of dry THF. 1.445 g (4.5 mmol) TBTU and 0.632 ml (4.5 mmol) triethylamine are added. The mixture is stirred at room temperature for one hour. 0.632 ml (4.5 mmol) triethylamine and 0.304 ml (4.5 mmol) azetidine are added. The mixture is stirred for 14 hours. After that time the mixture is diluted with water and extracted with EtOAc. The organic layer is dried over sodium sulphate. The product is obtained by filtration followed by evaporation of the solvent.

Yield: 1.2 g (100% of theory),

C₁₄H₂₄N₂O₃

EII Mass spectrum: m/z=269 [M+H]⁺

### II.1.b

### Azetidin-1-yl-piperidin-4-yl-methanone

A mixture of 1.5 g (5.59 mmol) 4-(azetidine-1-carbonyl)-piperidine-1-carboxylic acid tert-butyl ester and 2.08 ml (28 mmol) trifluoro acetic acid in 20 ml of methylene chloride is stirred for 14 hours at room temperature. The mixture is concentrated. Saturated potassium carbonate solution is added to the residue and the resulting mixture is stirred for 30 minutes. EtOAc is added. The water phase is concentrated, methylene chloride is added. This mixture is passed through a column (StratoSpheres SPE PL-HCO3 MP Resin). The filtrate is concentrated.

Yield: 0.1 g (10% of theory),

C₉H₁₆N₂O

EII Mass spectrum: m/z=169 [M+H]⁺

### Example II.2

### N-Methyl-N-piperidin-4-yl-acetamide

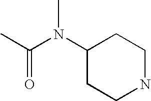

### II.2.a

### 4-(Benzyl-methyl-amino)-piperidine-1-carboxylic acid tert-butyl ester

A mixture of 5.05 g (17.38 mmol) of 4-benzylamino-piperidine-1-carboxylic acid tert-butyl ester, 3 ml (40.03 mmol) of formaldehyde (37% in water), 2 ml (34.97 mmol) acetic acid in 200 ml of methylene chloride is treated with 7.8 g (34.96 mmol) of sodium triacetoxyborohydride (95%) in portions. After stirring overnight potassium carbonate solution (15%) is added to the reaction mixture. The mixture is stirred for 30 minutes. The organic phase is separated and the aqueous phase is extracted with methylene chloride. The organic phases are combined, extracted with potassium carbonate solution and water. The organic phase is dried and concentrated.

Yield: 4.5 g (85% of theory),

C₁₈H₂₈N₂O₂

EII Mass spectrum: m/z=305 [M+H]⁺

### II.2.b

### 4-Methylamino-piperidine-1-carboxylic acid tert-butyl ester

A mixture of 4.5 g (14.78 mmol) 4-(benzyl-methyl-amino)-piperidine-1-carboxylic acid tert-butyl ester and 1 g of Pd/C (10%) in 100 ml of methanol is hydrogenated at 50° C. and 5 bar. The reaction mixture is filtered and the filtrate concentrated.

Yield: 2.95 g (93% of theory),

C₁₁H₂₂N₂O₂

EII Mass spectrum: m/z=215 [M+H]⁺

### II.2.c

### 4-(Acetyl-methyl-amino)-piperidine-1-carboxylic acid tert-butyl ester

0.971 ml (10.26 mmol) of acetic acid anhydride are added to a mixture of 2 g (9.32 mmol) of 4-methylamino-piperidine-1-carboxylic acid tert-butyl ester and 15 ml of acetic acid. The mixture is stirred overnight. Afterwards the mixture is poured in water. 2N sodium hydroxide solution is added until the mixture is basic. Then the mixture is extracted with EtOAc. The organic phase is extracted three times with water, dried an concentrated.

Yield: 1.2 g (50% of theory),

C₁₃H₂₄N₂O₃

EII Mass spectrum: m/z=257 [M+H]⁺

### II.2.d

### N-Methyl-N-piperidin-4-yl-acetamide trifluoroacetate

1.16 ml (15.05 mmol) of trifluoroacetic acid are added to a solution of 1.2 g (4.68 mmol) 4-(acetyl-methyl-amino)-piperidine-1-carboxylic acid tert-butyl ester in 20 ml methylene chloride. The reaction mixture is stirred for 14 hours at room temperature. The reaction mixture is concentrated and toluene is added. The solvent is removed.

Yield: 1.2 g (92% of theory)

C₈H₁₆N₂O.C₂HF₃O₂

EII Mass spectrum: m/z=157 [M+H]⁺

### Example II.3

### Piperidin-4-one O-methyl-oxime

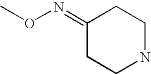

### II.3.a

### Piperidin-4-one O-methyl-oxime

A mixture of 3.84 g (25 mmol) of 4-piperidone-hydrate-hydrochloride and 2.506 g (83.52 mmol) of O-methyl-hydroxylamine-hydrochloride in 50 ml methanol are heated in a microwave oven to 60° C. at 300 W for one hour and 30 minutes. After cooling down saturated potassium carbonate solution is added and the reaction mixture is extracted with methylene chloride. and The organic phase is separated, dried and concentrated.

Yield: 1.7 g (53% of theory),

C₆H₁₂N₂O

EII Mass spectrum: m/z=129 [M+H]⁺

### Example II.4

### N-Methyl-N-piperidin-4-ylmethyl-acetamide trifluoroacetate

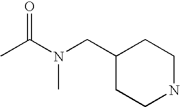

### II.4.a

### 4-Methylcarbamoyl-piperidine-1-carboxylic acid tert-butyl ester

To a solution of 6 g (26.17 mmol) piperidine-1,4-dicarboxylic acid mono-tert-butyl ester in 30 ml of dry THF are added 8.477 g (26.4 mmol) of TBTU and 3.7 ml (26.4 mmol) of triethylamine. The reaction mixture is stirred at room temperature for one hour. 3.7 ml (26.4 mmol) of triethylamine and 13.5 ml (26.4 mmol) of methylamine solution (2M) are added. The reaction mixture is stirred for 48 hours, diluted with water and extracted with EtOAc. The organic phase is dried over sodium sulphate.

Yield: 5.8 g (92% of theory),

C₁₂H₂₂N₂O₃

EII Mass spectrum: m/z=243 [M+H]⁺

### II.4.b

### 4-Aminomethyl-piperidine-1-carboxylic acid tert-butyl ester

5.76 g (16.64 mmol) of 4-methylcarbamoyl-piperidine-1-carboxylic acid tert-butyl ester are dissolved in 60 ml of dry THF. This solution is added to a suspension of 1.4 g (37 mmol) sodiumborohydride in 60 ml of THF at 0° C. and stirred for 30 minutes. A solution of 4.2 g (16.54 mmol) iodine in 60 ml of THF is added. The reaction mixture is heated to reflux for 18 hours. Afterwards the reaction mixture is cooled to room temperature and 150 ml MeOH are added dropwise. The reaction mixture is concentrated and sodiumhydroxid solution is added. The reaction mixture is extracted with tert.butyl methyl ether. The organic phase is dried over sodium sulphate.

Yield: 5 g (60% of theory),

C₁₂H₂₄N₂O₂

EII Mass spectrum: m/z=229 [M+H]⁺

### II.4.c

### 4-[(Acetyl-methyl-amino)-methyl]-piperidine-1-carboxylic acid tert-butyl ester

To a solution of 5.06 g (13.29 mmol) 4-aminomethyl-piperidine-1-carboxylic acid tert-butyl ester in methylene chloride are added at 0° C. 1.3 ml (16.43 mmol) pyridine and 1.15 ml (16.18 mmol) acetyl chloride. The reaction mixture is stirred at room temperature for 18 hours. After that time water is added. The organic phase is collected by passing the reaction mixture through a column (Phase Separator/Separtis). The organic phase is concentrated. The residue is purified by silica gel column chromatography with methylene chloride/MeOH as eluent.

Yield: 2 g (37% of theory),

C₁₄H₂₆N₂O₃

EII Mass spectrum: m/z=271 [M+H]⁺

### II.4.d

### N-Methyl-N-piperidin-4-ylmethyl-acetamide trifluoroacetate

A reaction mixture of 2 g (6.65 mmol) 4-[(acetyl-methyl-amino)-methyl]-piperidine-1-carboxylic acid tert-butyl ester and 2.6 ml (35 mmol) trifluoracetic acid in 20 ml of methylene chloride is stirred at room temperature for 24 hours. The reaction mixture is concentrated and toluene is added. The reaction mixture is concentrated again.

Yield: 1.9 g (100% of theory),

C₉H₁₈N₂O.C₂HF₃O₂

EII Mass spectrum: m/z=171 [M+H]⁺

### Example II.5

### N,N-Dimethyl-2-piperidin-4-yl-acetamide trifluoroacetate

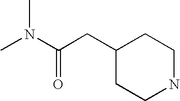

### II.5.a

### 4-Dimethylcarbamoylmethyl-piperidine-1-carboxylic acid tert-butyl ester

To a solution of 2 g (8.22 mmol) 4-carboxymethyl-piperidine-1-carboxylic acid tert-butyl ester and 3.481 ml (25 mmol) of triethylamine in 100 ml of dry THF are added 2.665 g (8.3 mmol) of TBTU. The reaction mixture is stirred at room temperature for 30 minutes. 4.3 ml (8.22 mmol) of 2 M dimethylamine solution in THF are added. The reaction mixture is stirred for 18 hours, concentrated and extracted with EtOAc and water. The organic phase is dried over sodium sulphate and concentrated. The residue is washed with diisopropylether.

Yield: 2.2 g (99% of theory),

R_{f} value: 0.45 (silica gel, methylene chloride/methanol/ammonia solution=90:10:1)

### II.5.b

### N,N-Dimethyl-2-piperidin-4-yl-acetamide trifluoroacetate

Prepared analogously to II.4.d from 4-dimethylcarbamoylmethyl-piperidine-1-carboxylic acid tert-butyl ester.

Yield: 2.29 g (99% of theory),

R_{f} value: 0.2 (silica gel, methylene chloride/methanol/ammonia solution=90:10:1)

### Example II.6

### (R)-Pyrrolidine-3-carboxylic acid dimethylamide trifluoroacetate

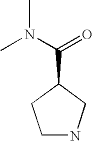

### II.6.a

### (R)-3-Dimethylcarbamoyl-pyrrolidine-1-carboxylic acid tert-butyl ester

Prepared analogously to II.5.a from (R)-pyrrolidine-1,3-dicarboxylic acid 1-tert-butyl ester and 2M dimethylamine solution in THF.

Yield: 5.6 g (83% of theory)

C₁₂H₂₂N₂O₃

EII Mass spectrum: m/z=243 [M+H]⁺

### II.6.b

### (R)-Pyrrolidine-3-carboxylic acid dimethylamide trifluoroacetate

Prepared analogously to II.4.d from (R)-3-dimethylcarbamoyl-pyrrolidine-1-carboxylic acid tert-butyl ester.

Yield: 4.87 g (82% of theory),

C₇H₁₄N₂O.C₂HF₃O₂

EII Mass spectrum: m/z=143 [M+H]⁺

### Example II.7

### (S)-Pyrrolidine-3-carboxylic acid dimethylamide trifluoroacetate

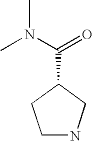

Prepared analogously to II.6.a to II.6.b starting from (S)-pyrrolidine-1,3-dicarboxylic acid 1-tert-butyl ester

Yield for Boc-deprotection: 4.47 g (62% of theory),

C₇H₁₄N₂O.C₂HF₃O₂

EII Mass spectrum: m/z=143 [M+H]⁺

### Example II.8

### N-(S)-1-Piperidin-3-ylmethyl-acetamide trifluoroacetate

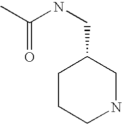

### II.8.a

### (R)-3-(Acetylamino-methyl)-piperidine-1-carboxylic acid tert-butyl ester

To a solution of 300 mg (1.4 mmol) (R)-3-aminomethyl-piperidine-1-carboxylic acid tert-butyl ester in 10 ml methylene chloride is first added 370 μl (2.1 mmol) triethylamine and then slowly 0.110 ml (1.68 mmol) acetyl chloride. The reaction mixture is stirred at room temperature for three hours. Water is added afterwards. The organic phase is dried over sodium sulphate and concentrated.

Yield: 370 mg (103% of theory)

C₁₃H₂₄N₂O₃

EII Mass spectrum: m/z=257 [M+H]⁺

### II.8.b

### N-(S)-1-Piperidin-3-ylmethyl-acetamide trifluoroacetate

Prepared analogously to II.4.d from (R)-3-(Acetylamino-methyl)-piperidine-1-carboxylic acid tert-butyl ester

Yield: 550 mg (149% of theory), raw material

C₈H₁₆N₂O.C₂HF₃O₂

EII Mass spectrum: m/z=157 [M+H]⁺

### Example III.1

### 6-[2-(6-Benzyloxy-pyridazin-3-yl)-ethyl]-pyridine-3-carbaldehyde

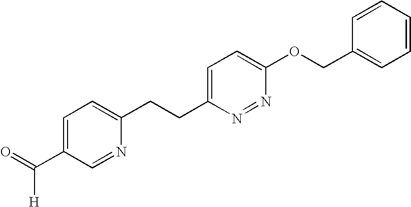

### III.1.a

### 3-Benzyloxy-6-trimethylsilanylethynyl-pyridazine

A mixture of 21.5 g (66 mmol) of cesium carbonate and 10 g (32.04 mmol) of 3-benzyloxy-6-iodo-pyridazine in 150 ml of dry THF is cooled to −15° C. The mixture is degassed and flushed with argon. Then 1.19 g (1.7 mmol) bis-(triphenylphosphine)-palladium dichloride and 324 mg (1.7 mmol) copper(I)-iodide are added. The resulting mixture is degassed as above and flushed with argon. 4.94 ml (35 mmol) ethynyl-trimethyl-silane are added and the mixture is stirred for 30 minutes at −15° C. and 18 hours at room temperature. After that time the reaction mixture is poured in water and concentrated ammonia solution is added. The mixture is extracted with EtOAc. The organic phase is extracted with saturated sodium chloride solution, dried over sodium sulphate and activated charcoal and concentrated.

Yield: 9.6 g (90% of theory),

C₁₆H₁₈N₂OSi

EII Mass spectrum: m/z=283 [M+H]⁺

### III.1.b

### 3-Benzyloxy-6-ethynyl-pyridazine

A reaction mixture of 6.3 g (22.3 mmol) 3-benzyloxy-6-trimethylsilanylethynyl-pyridazine and 50 ml of 1M sodiumhydroxid solution in 300 ml methanol is stirred for five hours at room temperature. Then a citric acid solution (10%) is added so that the pH is 6. The reaction mixture is concentrated. The residue is diluted with water. The solid is collected and dried. Purification is achieved by silica gel column chromatography with methylene chloride/MeOH as eluent.

Yield: 3.1 g (66% of theory),

C₁₃H₁₀N₂O

EII Mass spectrum: m/z=211 [M+H]⁺

R_{f} value: 0.7 (silica gel, methylene chloride/ethanol=50:1)

### III.1.c

### 6-(6-Benzyloxy-pyridazin-3-ylethynyl)-pyridine-3-carbaldehyde

A mixture of 1.4 g (7.52 mmol) 6-bromo-pyridine-3-carbaldehyde and 5.7 ml (40.33 mmol) diisopropylamine in 60 ml dry THF is degassed as described in example III1.a. 280 mg (0.4 mmol) of bis-(triphenylphosphin)-palladium-II-chloride and 77 mg (0.4 mmol) of copper iodide are added and the reaction mixture is degassed again. Then 1.8 g (8.56 mmol) of 3-benzyloxy-6-ethynyl-pyridazine is added and the mixture is stirred for 18 hours. The mixture is poured on water and the solid is collected. The solid is washed with diisopropylether and dried.

Yield: 1.3 g (54.8% of theory),

C₁₉H₁₃N₃O₂

EII Mass spectrum: m/z=316 [M+H]⁺

### III.1.d

### 6-[2-(6-Benzyloxy-pyridazin-3-yl)-ethyl]-pyridine-3-carbaldehyde and {6-[2-(6-Benzyloxy-pyridazin-3-yl)-ethyl]-pyridazin-3-yl}-methanol

A mixture of 2.8 g (8.88 mmol) 6-(6-benzyloxy-pyridazin-3-ylethynyl)-pyridine-3-carbaldehyde and 0.6 g Raney/Ni in 100 ml dry DMF is stirred under hydrogen atmosphere (40-50 psi) at room temperature for two days. When the reduction is completed the reaction mixture filtered and concentrated. The residue is purified by silica gel column chromatography with methylene chloride/MeOH and ammonia solution as eluent.

### 6-[2-(6-Benzyloxy-pyridazin-3-yl)-ethyl]-pyridine-3-carbaldehyde

Yield: 0.7 g (24.7% of theory),

C₁₉H₁₇N₃O₂

EII Mass spectrum: m/z=320 [M+H]⁺

### {6-[2-(6-Benzyloxy-pyridazin-3-yl)-ethyl]-pyridazin-3-yl}-methanol

Yield: 0.8 g (28% of theory),

C₁₉H₁₉N₃O₂

EII Mass spectrum: m/z=322 [M+H]⁺

### Example III.2

### 3-Benzyloxy-6-[2-(5-chloromethyl-pyridin-2-yl)-ethyl]-pyridazine

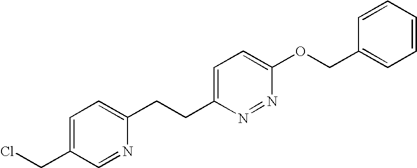

### III.2.a

### 3-Benzyloxy-6-[2-(5-chloromethyl-pyridin-2-yl)-ethyl]-pyridazine

0.054 ml (0.7 mmol) methanesulfonyl chloride is added to a solution of 0.22 g (0.68 mmol) {6-[2-(6-benzyloxy-pyridazin-3-yl)-ethyl]-pyridazin-3-yl}-methanol and 0.126 ml (0.9 mmol) triethylamine in 6 ml methylene chloride. The reaction mixture is stirred for 24 hours at room temperature. The reaction mixture is extracted with water. The organic phase is collected by passing the reaction mixture through a column (Phase Separator/Separtis). The organic phase is concentrated and used without further purification

Yield: 0.24 g,

C₁₉H₁₈CIN₃O

EII mass spectrum: m/z=340/342 [M+H]⁺

### Example III.3

### 3-[2-(4-Chloromethyl-phenyl)-ethyl]-6-(1-phenyl-ethoxy)-pyridazine

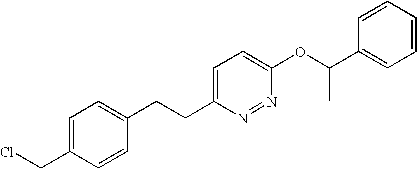

### III.3.a

### 3-Iodo-6-(1-phenyl-ethoxy)-pyridazine

417 mg (9.55 mmol) sodium hydride (55%) are added to a solution of 1-phenyl-ethanol in 70 ml THF. The reaction mixture is stirred for 30 minutes at room temperature. Then 2.882 g (8.68 mmol) 3,6-diiodo-pyridazine are added. The reaction mixture is stirred for 18 hours at room temperature. After that time the reaction mixture is poured into water and extracted with EtOAc. The organic phase is extracted with water three times, dried over sodium sulphate and concentrated. The residue is purified by silica gel column chromatography with cyclohexan/EtOAc as eluent.

Yield: 2.45 g (86.5% of theory),

retention time (HPLC): 3.27 min (method A)

C₁₂H₁₁IN₂O

EII mass spectrum: m/z=327 [M+H]⁺

### III.3.b

### 4-[6-(1-Phenyl-ethoxy)-pyridazin-3-ylethynyl]-benzaldehyde

Prepared analogously to 1.1.c from 3-iodo-6-(1-phenyl-ethoxy)-pyridazine and 4-ethynyl-benzaldehyde.

Yield: 1.7 g (69% of theory),

C₂₁H₁₆N₂O₂

EII mass spectrum: m/z=329 [M+H]⁺

### III.3.c

### (4-{2-[6-(1-Phenyl-ethoxy)-pyridazin-3-yl]-ethyl}-phenyl)-methanol

A mixture of 1.7 g (5.17 mmol) 4-[6-(1-Phenyl-ethoxy)-pyridazin-3-ylethynyl]-benzaldehyde and 300 mg of raney-nickel in 80 ml of EtOAc and 70 ml methanol is stirred for 9 hours at RT in a hydrogen atmosphere (50 psi). The mixture is filtrated, The solvent is evaporated. The residue is purified by silica gel column chromatography with cyclohexane/EtOAc as eluent.

Yield: 0.9 g (52% of theory),

C₂₁H₂₂N₂O₂

EII mass spectrum: m/z=335 [M+H]⁺

### III.3.d

### 3-[2-(4-Chloromethyl-phenyl)-ethyl]-6-(1-phenyl-ethoxy)-pyridazine

0.205 ml (2.64 mmol) methanesulfonyl chloride is added to a solution of 0.9 g (2.69 mmol) (4-{2-[6-(1-phenyl-ethoxy)-pyridazin-3-yl]-ethyl}-phenyl)-methanol and 0.757 ml (5.38 mmol) triethylamine in 70 ml methylene chloride. The reaction mixture is stirred for 3 hours at room temperature. The reaction mixture is extracted with water three times. The organic phase is dried over sodium sulphate and concentrated.

Yield: 0.8 g (84% of theory),

retention time (HPLC): 3.85 min (method A)

C₂₁H₂₁CIN₂O

EII mass spectrum: m/z=351/353 [M+H]⁺

### Example III.4

### 3-Benzyloxy-6-[2-(6-chloromethyl-pyridin-3-yl)-ethyl]-pyridazine

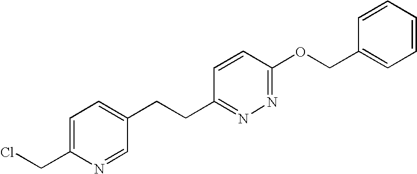

### III.4.a (5-Trimethylsilanylethynyl-pyridin-2-yl)-methanol

Prepared analogously to example III.1.a from (5-Iodo-pyridin-2-yl)-methanol and Ethynyl-trimethyl-silane.

Yield: 3.3 gg (80% of theory),

C₁₁H₁₅NOSi

EII mass spectrum: m/z=206 [M+H]⁺

### III.4.b (5-Ethynyl-pyridin-2-yl)-methanol

Prepared analogously to example III.1.b from (5-Trimethylsilanylethynyl-pyridin-2-yl)-methanol.

Yield: 1.9 g (89% of theory),

### III.4.c [5-(6-Benzyloxy-pyridazin-3-ylethynyl)-pyridin-2-yl]-methanol

Prepared analogously to example III.1.c from (5-Ethynyl-pyridin-2-yl)-methanol and 3-Benzyloxy-6-iodo-pyridazine.

Yield: 0.95 g (20% of theory),

C₁₉H₁₅N₃O₂

EII mass spectrum: m/z=318 [M+H]⁺

### III.4.d {5-[2-(6-Benzyloxy-pyridazin-3-yl)-ethyl]-pyridin-2-yl}-methanol

Prepared analogously to example III.1.d from [5-(6-Benzyloxy-pyridazin-3-ylethynyl)-pyridin-2-yl]-methanol.

Yield: 0.15 g (20% of theory),

C₁₉H₁₉N₃O₂

EII mass spectrum: m/z=322 [M+H]⁺

### III.4.e 3-Benzyloxy-6-[2-(6-chloromethyl-pyridin-3-yl)-ethyl]-pyridazine

A mixture of 0.1 g (0.31 mmol) {5-[2-(6-Benzyloxy-pyridazin-3-yl)-ethyl]-pyridin-2-yl}-methanol and 0.5 ml thionylchloride in 20 ml methylene chloride is stirred at room temperature for two hours. Sodium hydroxide solution (2N) is added. The organic phase is separated and dried over sodium sulphate.

Yield: 0.06 g (56% of theory),

### Example III.5

### 3-[2-(5-Chloromethyl-pyridin-2-yl)-ethyl]-6-phenethyl-pyridazine

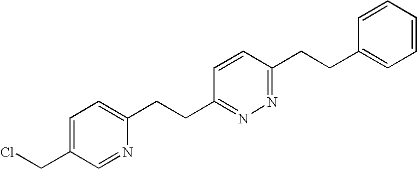

### III.5.a 3-Iodo-6-phenethyl-pyridazine

To 200 ml (100 mmol) 0.5 M phenylethylzincbromid solution in THF are added dropwise a mixture of 26.55 g (80 mmol) 3.6 Diiodopyridazine and 4.622 g (4 mmol) Tetrakis(triphenylphosphine) palladium(0) in 100 ml abs. THF. The reaction mixture is stirred for 3 hours at room temperature. Then the mixture is poured in saturated sodium hydrogencarbonate solution and ethyl acetate is added. The mixture is filtered over Celite and the phases are separated. The organic phase is dried over sodium sulphate. Purification is achieved by chromatography (silica gel, methylene chloride/ethyl acetate=19:1).

Yield: 13.97 g (56% of theory),

R_{f} value: 0.47 (silica gel, methylene chloride/ethyl acetate=19:1)

C₁₂H₁₁IN₂

EII mass spectrum: m/z=311 [M+H]⁺

### III.5.b 3-Phenethyl-6-trimethylsilanylethynyl-pyridazine

Prepared analogously to example III.1.a from 3-Iodo-6-phenethyl-pyridazine and Ethynyl-trimethyl-silane.

Yield: 13 g

R_{f} value: 0.73 (silica gel, petrolether/ethyl acetate=1:1)

C₁₇H₂₀N₂Si

EII mass spectrum: m/z=281 [M+H]⁺

### III.5.c 3-Ethynyl-6-phenethyl-pyridazine

Prepared analogously to example III.1.b from 3-Phenethyl-6-trimethylsilanylethynyl-pyridazine.

Yield: 5.84 g (60% of theory),

R_{f} value: 0.6 (silica gel, petrolether/ethyl acetate=1:1)

C₁₄H₁₂N₂

EII mass spectrum: m/z=209 [M+H]⁺

### III.5.d 6-(6-Phenethyl-pyridazin-3-ylethynyl)-pyridine-3-carbaldehyde

Prepared analogously to example III.1.c from 3-Ethynyl-6-phenethyl-pyridazine and 6-Bromo-pyridine-3-carbaldehyde.

Yield: 6 g (72% of theory),

R_{f} value: 0.57 (silica gel, methylene chloride/methanol/ammonia solution=90:10:1)

C₂₀H₁₅N₃O

EII mass spectrum: m/z=314 [M+H]⁺

### III.5.e{6-[2-(6-Phenethyl-pyridazin-3-yl)-ethyl]-pyridin-3-yl}-methanol

Prepared analogously to example III.1.d from 6-(6-Phenethyl-pyridazin-3-ylethynyl)-pyridine-3-carbaldehyde.

Yield: 1.23 g (22% of theory),

R_{f} value: 0.35 (silica gel, methylene chloride/methanol/ammonia solution=90:10:1)

C₂₀H₂₁N₃O

EII mass spectrum: m/z=320 [M+H]⁺

### III.5.f 3-[2-(5-Chloromethyl-pyridin-2-yl)-ethyl]-6-phenethyl-pyridazine

Prepared analogously to example III.2.a from {6-[2-(6-Phenethyl-pyridazin-3-yl)-ethyl]-pyridin-3-yl}-methanol.

Yield: 0.46 g (35% of theory),

R_{f} value: 0.45 (silica gel, methylene chloride/methanol/ammonia solution=90:10:1)

### Example III.6

### 3-Benzyloxy-6-{2-[5-(2-chloro-ethyl)-pyridin-2-yl]-ethyl}-pyridazine

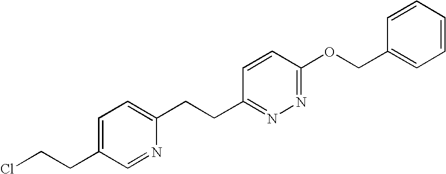

### III.6.a (6-Iodo-pyridin-3-yl)-acetic acid

30 g (174.85 mmol) 6-Chloro-pyridin-3-yl)-acetic acid is dissolved in 750 ml acetonitrile. 524, 315 g (3498 mmol) sodium iodide are added. Then 36 ml concentrated hydrochloric acid (32%) are added dropwise. The mixture is refluxed for 48 hours. The mixture is evaporated and 2 l water are added. The precipitate is collected by filtration and dried. Purification is achieved by chromatography (silica gel, methylene chloride/ethyl acetate=19:1).

Yield: 40 g (87% of theory),

C₇H₆INO₂

EII mass spectrum: m/z=264 [M+H]⁺

### III.6.b (6-Iodo-pyridin-3-yl)-acetic acid methyl ester

87.44 ml (174.88 mmol) Trimethylsiliyldiazomethane solution (2M) are added dropwise to a solution of 40 g (152.07 mmol) 6-Iodo-pyridin-3-yl)-acetic acid in 810 ml ethyl acetate and 90 ml methanol so that the temperature is no exceeding 30° C. Then the mixture is stirred until no further gas evolution is observed. After that time the mixture is evaporated to dryness.

Yield: 42 g (100% of theory),

C₈H₈INO₂

EII mass spectrum: m/z=278 [M+H]⁺

### III.6.c [6-(6-Benzyloxy-pyridazin-3-ylethynyl)-pyridin-3-yl]acetic acid methyl ester

Prepared analogously to example III.1.c from (6-Iodo-pyridin-3-yl)-acetic acid methyl ester and 3-benzyloxy-6-ethynyl-pyridazine.

Yield: 4.8 g (61% of theory),

C₂₁H₁₇N₃O₃

EII mass spectrum: m/z=360 [M+H]⁺

### III.6.d {6-[2-(6-Benzyloxy-pyridazin-3-yl)-ethyl]-pyridin-3-yl}-acetic acid methyl ester

Prepared analogously to example III.1.d from [6-(6-Benzyloxy-pyridazin-3-ylethynyl)-pyridin-3-yl]-acetic acid methyl ester.

Yield: 2.6 g (85.7% of theory),

C₂₁H₂₁N₃O₃

EII mass spectrum: m/z=364 [M+H]⁺

### III.6.e 2-{6-[2-(6-Benzyloxy-pyridazin-3-yl)-ethyl]-pyridin-3-yl}-ethanol

1.63 g (4.48 mmol) {6-[2-(6-Benzyloxy-pyridazin-3-yl)-ethyl]-pyridin-3-yl}-acetic acid methyl ester are dissolved in 20 ml THF. Then 2 ml (2 mmol) Lithiumhydride solution (2M) are added and the mixture is stirred for one hour at room temperature. Water is added carefully. Then celite is added to the mixture. The mixture is filtrated. The filtrate is evaporated. The residue is taken up in ethyl acetate. The resulting mixture is extracted with water. The organic phase is dried over sodium sulphate and evaporated.

Yield: 1.35 g (90% of theory),

C₂₀H₂₁N₃^{O}₂

EII mass spectrum: m/z=336 [M+H]⁺

### III.6.f 3-Benzyloxy-6-{2-[5-(2-chloro-ethyl)-pyridin-2-yl]-ethyl}-pyridazine

2.7 g (8.05 mmol) 2-{6-[2-(6-Benzyloxy-pyridazin-3-yl)-ethyl]-pyridin-3-yl}-ethanol are dissolved in methylene chloride. 3.167 g (12.07 mmol) Triphenylphosphine and 1.223 ml (8.05 mmol) Hexachloroacetone are added. The mixture is stirred for four hours at room temperature. 50 ml methylene chloride are added and the resulting mixture is extracted with water. The organic phase is dried over sodium sulphate. Purification is achieved by chromatography (silica gel, cyclohexane/ethyl acetate=1:1-1:4), followed by HPLC (method 3).

Yield: 1.1 g (90% of theory),

retention time (HPLC): 2.30 min (method A)

C₂₀H₂₀CIN₃O

EII mass spectrum: m/z=354 [M+H]⁺

### Example III.7

### Methanesulfonic acid 2-{6-[2-(6-benzyloxy-pyridazin-3-yl)-ethyl]pyridin-3-yl}-ethyl ester

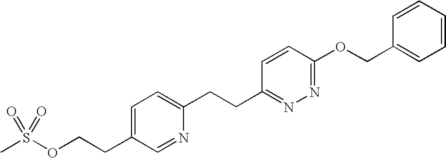

Prepared analogously to example IV.2.f from 2-{6-[2-(6-Benzyloxy-pyridazin-3-yl)-ethyl]-pyridin-3-yl}-ethanol and methane sulfonyl chloride.

Yield: 0.9 g (73% of theory),

### Example III.8

### Methanesulfonic acid 2-{3-[2-(6-benzyloxy-pyridazin-3-yl)-ethyl]-phenyl}-ethyl ester

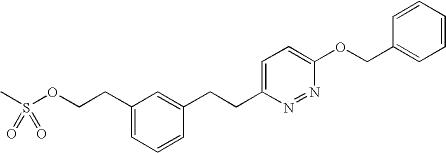

### III.8.a 2-(3-iodophenyl)ethanol

A solution of 9.3 g (33.7 mmol) (3-iodophenyl)acetic acid is dissolved in 160 ml dry THF and cooled to 0° C. Then 34.0 ml of a 1.0 M solution of lithium aluminumhydride in THF (34.0 mmol) is slowly added. The reaction mixture is allowed to warm to room temperature and stirred for 2 hours. The excess lithium aluminumhydride is carefully destroyed with a few drops of water and the resulting mixture is filtered through celite. The solvent is removed in vacuo leaving the crude product, which is used without further purification in the next step.

Yield: 6.83 g (81% of theory),

C₈H₉IO

EII mass spectrum: m/z=249 [M+H]⁺

### III.8.b 243-(6-Benzyloxy-pyridazin-3-ylethynyl)-phenyl-ethanol

A solution of 1.18 g (4.76 mmol) 2-(3-iodophenyl)ethanol in 40 ml dry THF is degassed as described in example III1.a. 200 mg (0.285 mmol) bis-(triphenylphosphin)-palladium-II-chloride and 60 mg (0.315 mmol) copper iodide are added and the reaction mixture is degassed again. Then 1.00 g (4.76 mmol) 3-benzyloxy-6-ethynyl-pyridazine and 1.5 ml (10.7 mmol) diisopropylamine are added and the mixture is stirred for 4 hours at room temperature. The mixture is poured onto water and extracted with dichloromethane. The organic layer is dried over sodium sulphate and concentrated. The residue is purified by silica gel column chromatography.

Yield: 1.6 g (99% of theory),

C₂₁H₁₈N₂^{O}₂

EII Mass spectrum: m/z=331 [M+H]⁺

### III.8.c 243-(6-Benzyloxy-pyridazin-3-ylethyl)-phenyl-ethanol

A mixture of 8.93 g (27.03 mmol) 2-[3-(6-benzyloxy-pyridazin-3-ylethynyl)-phenyl]-ethanol and 1.0 g Raney/Ni in 220 ml dry DMF is stirred under hydrogen atmosphere (40-50 psi) at room temperature for 24 hours. When the reduction is completed the reaction mixture is filtered and concentrated. The residue is treated with water and ethyl acetate and the layers are separated. The organic layer is dried over sodium sulphate and concentrated. The residue is purified by silica gel column chromatography with petrolether/ethyl acetate (1:1) and methylene chloride/MeOH/0.1% ammonia as eluents. The solid is washed with diisopropylether and dried.

Yield: 2.0 g (22% of theory),

C₂₁H₂₂N₂O₂

EII Mass spectrum: m/z=335 [M+H]

### III.8.d Methanesulfonic acid 2-[3-(6-benzyloxy-pyridazin-3-ylethyl)-phenyl]-ethyl ester

To a solution of 2.0 g (5.98 mmol) 2-[3-(6-benzyloxy-pyridazin-3-ylethyl)-phenyl]-ethanol and 1.10 ml (7.84 mmol) triethylamine in 65 ml methylene chloride are dropwise added 0.509 ml (6.58 mmol) methylsulfonylchloride. After stirring for 1 hour at room temperature the mixture is treated with water and extracted with methylene chloride. The organic layer is dried over sodium sulphate and concentrated in vacuo. The solid is washed with diisopropylether and dried.

Yield: 2.33 g (93% of theory),

C₂₂H₂₄N₂O₄S

EII mass spectrum: m/z=413 [M+H]⁺

### Example III.9

### 3-Benzyloxy-6-{2-[4-(2-chloro-ethyl)-phenyl]-ethyl}-pyridazine

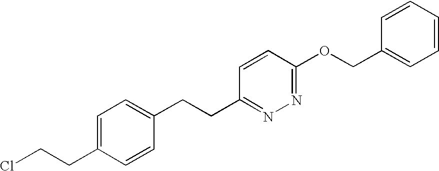

### III.9.a 2-(4-Iodo-phenyl)-ethanol

Prepared analogously to example V.5.a from 2-(4-Bromo-phenyl)-ethanol.

Yield: 5.9 g (90% of theory),

C₈H₉IO

EII mass spectrum: m/z=249 [M+H]⁺

### III.9.b 2-[4-(6-Benzyloxy-pyridazin-3-ylethynyl)-phenyl]-ethanol

Prepared analogously to example III.1.c from 2-(4-Iodo-phenyl)-ethanol and 3-benzyloxy-6-ethynyl-pyridazine.

Yield: 1.7 g (45% of theory),

C₂₁H₁₈N₂O₂

EII mass spectrum: m/z=331 [M+H]⁺

### III.9.c 2-{4-[2-(6-Benzyloxy-pyridazin-3-yl)-ethyl]-phenyl}-ethanol

Prepared analogously to example III.1.d from 2-[4-(6-Benzyloxy-pyridazin-3-ylethynyl)-phenyl]-ethanol.

Yield: 1.9 g (88% of theory),

C₂₁H₂₂N₂^{O}₂

EII mass spectrum: m/z=335 [M+H]⁺

### III.9.d 3-Benzyloxy-6-{2-[4-(2-chloro-ethyl)-phenyl]-ethyl}-pyridazine

2.2 g (5.26 mmol) 2-{4-[2-(6-Benzyloxy-pyridazin-3-yl)-ethyl]-phenyl}-ethanol are dissolved in 50 ml methylene chloride. 0.386 ml (5.3 mmol) thionyl chloride are added. The mixture is stirred for 18 hours and extracted with water. The organic phase is dried over sodium sulphate. Purification is achieved by chromatography (silica gel, methylen chloride/methanol=100:1).

Yield: 1.35 g (90% of theory),

C₂₁H₂₁CIN₂O

EII mass spectrum: m/z=353/355 [M+H]⁺

### Example III.10

### Methanesulfonic acid 2-{4-[2-(6-benzyloxy-pyridazin-3-yl)-ethyl]-phenyl}-ethyl ester

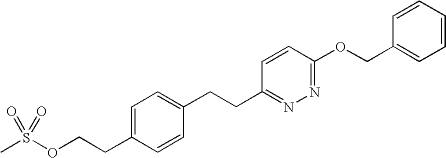

Prepared analogously to example IV.2.f from 2-{4-[2-(6-Benzyloxy-pyridazin-3-yl)-ethyl]-phenyl}-ethanol and methane sulfonylchloride.

Yield: 1 g (53% of theory),

C₂₂H₂₄N₂O₄S

EII mass spectrum: m/z=413 [M+H]⁺

### Example IV.1

### 3-Iodo-6-phenoxy-pyridazine

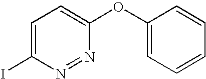

### IV.1.a

### 3-Iodo-6-phenoxy-pyridazine

A mixture of 1.242 g (13.2 mmol) phenol and 1.8 g (13.2 mmol) potassium carbonate in 100 ml dry acetonitrile is refluxed for 15 minutes. 3.651 g (11 mmol) 3,6-diiodo-pyridazine are added and the reaction mixture is refluxed for 24 hours. After that time the reaction mixture is filtered. The filtrate is concentrated. The residue is purified by silica gel column chromatography with petrol ether/EtOAc as eluent.

Yield: 3.1 g (94.5% of theory),

C₁₀H₇IN₂O

EII Mass spectrum: m/z=299 [M+H]⁺

R_{f} value: 0.6 (silica gel, petrole ether/EtOAc=5:2)

### Example IV.2

### Methanesulfonic acid 2-{4-[2-(6-phenoxy-pyridazin-3-yl)-ethyl]-phenoxy}-ethyl ester

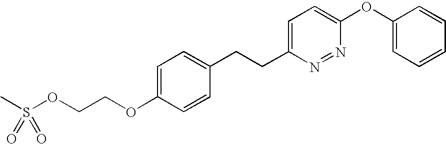

### IV.2.a

### 2-(4-Iodo-phenoxy)-ethanol

A reaction mixture of 50 g (0.22 mol) 4-iodo-phenol, 18.298 ml (0.27 mol) 2-chloro-ethanol and 125.64 g (0.91 mol) potassium carbonate in 500 ml DMF is stirred at 80° C. for 16 hours. 3.6 ml 2-chloro-ethanol are added and the reaction mixture is stirred for another two hours at 80° C. Then the reaction mixture is concentrated. 500 ml EtOAc are added to the residue and the resulting mixture is extracted with 400 ml of water and two times with 350 ml of 1 M sodium hydroxide solution. The organic phase is dried over magnesium sulphate and concentrated.

Yield: 57.3 g (95% of theory),

R_{f} value: 0.5 (silica gel, petrole ether/EtOAc=1:1)

### IV.2.b

### 2-(4-Trimethylsilanylethynyl-phenoxy)-ethanol

To a solution of 26.406 g (10 mmol) 2-(4-iodo-phenoxy)-ethanol in 500 ml dry THF are added under argon 1.404 g (2 mmol) bis-(triphenylphosphine)-palladium-II-chloride, 381 mg (2 mmol) copper-iodide and 40 ml (287 mmol) triethylamine. 15.238 ml (11 mmol) ethynyl-trimethyl-silane are added dropwise to this reaction mixture. The reaction mixture is stirred for 1.5 hours at a temperature between 20 and 25° C. After that time the reaction mixture is concentrated. The residue is taken up in 400 ml EtOAc and extracted with 500 ml water. The organic phase is dried over magnesium sulphate and concentrated.

Yield: 27.2 g (99% of theory),

R_{f} value: 0.5 (silica gel, petrole ether/EtOAc=1:1)

### IV.2.c

### 2-(4-Ethynyl-phenoxy)-ethanol

27.949 g (10 mmol) tetrabutyl ammonium fluoride are added to a solution of 27.2 g (98.64 mmol) 2-(4-trimethylsilanylethynyl-phenoxy)-ethanol in 350 ml methylene chloride and the reaction mixture is stirred for one hour at room temperature. The reaction mixture is extracted two times with 300 ml of water. The organic phase is dried over magnesium sulphate and concentrated. Purification is achieved by silica gel column chromatography with petrole ether/EtOAc as eluent.

Yield: 11.7 g (73% of theory),

R_{f} value: 0.5 (silica gel, petrole ether/EtOAc=1:1)

### IV.2.d

### 2-[4-(6-Phenoxy-pyridazin-3-ylethynyl)-phenoxy]-ethanol

A solution of 1 g (3.35 mmol) 3-iodo-6-phenoxy-pyridazine dry 35 ml THF is degassed. Under an argon atmosphere 130 mg (0.18 mmol) bis-(triphenylphosphine)-palladium-II-chloride, 0.854 ml (6.09 mmol) diisopropylamine and 55 mg (0.29 mmol) copper-iodide are added. The reaction mixture is degassed and set under an argon atmosphere again. 568 mg (3.5 mmol) 2-(4-ethynyl-phenoxy)-ethanol are added and the reaction mixture is stirred for two hours at room temperature. The reaction mixture is concentrated and water is added. The solid is filtered, washed with diisopropylether and dried. Purification is achieved by silica gel column chromatography with methylene chloride/methanol as eluent.

Yield: 0.92 g (82% of theory),

C₂₀H₁₆N₂O₃

EII mass spectrum: m/z=333 [M+H]⁺

### IV.2.e

### 2-{4-[2-(6-Phenoxy-pyridazin-3-yl)-ethyl]-phenoxy}-ethanol

Prepared analogously to 11.1.d from 2-[4-(6-phenoxy-pyridazin-3-ylethynyl)-phenoxy]-ethanol.

Yield: 0.82 g (75% of theory),

C₂₀H₂₀N₂O₃

EII mass spectrum: m/z=337 [M+H]⁺

### IV.2.f

### Methanesulfonic acid 2-{4-[2-(6-phenoxy-pyridazin-3-yl)-ethyl]-phenoxy}-ethyl ester

0.201 ml (2.6 mmol) methanesulfonyl chloride is added to a solution of 0.82 g (2.44 mmol) 2-{4-[2-(6-phenoxy-pyridazin-3-yl)-ethyl]-phenoxy}-ethanol and 0.702 ml (5 mmol) triethylamine in 20 ml methylene chloride. The reaction mixture is stirred for 48 hours at room temperature. Water is added. The organic phase is collected by passing the reaction mixture through a column (Phase Separator/Separtis). The organic phase is concentrated.

Yield: 0.9 g (89% of theory),

C₂₁H₂₂N₂O₅S

EII mass spectrum: m/z=415 [M+H]⁺

R_{f} value: 0.5 (silica gel, methylene chloride/EtOH=20:1)

### Example IV.3

### Methanesulfonic acid 2-{4-[2-(6-benzyloxy-pyridazin-3-yl)-ethyl]-phenoxy}-ethyl ester

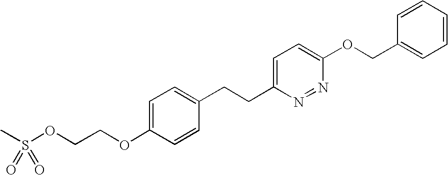

### IV.3.a

### 2-[4-(6-Benzyloxy-pyridazin-3-ylethynyl)-phenoxy]-ethanol

Prepared analogously to IV.2.d from 3-benzyloxy-6-iodo-pyridazine and 2-(4-ethynyl-phenoxy)-ethanol.

Yield: 1.2 g (100% of theory),

C₂₁H₁₈N₂O₃

EII mass spectrum: m/z=347 [M+H]⁺

### IV.3.b

### 2-{4-[2-(6-benzyloxy-pyridazin-3-yl)-ethyl]-phenoxy}-ethanol

Prepared analogously to 1.1.d from 2-[4-(6-benzyloxy-pyridazin-3-ylethynyl)-phenoxy]-ethanol.

Yield: 0.6 g (49% of theory),

C₂₁H₂₂N₂O₃

EII mass spectrum: m/z=351 [M+H]⁺

### IV.3.c.

### Methanesulfonic acid 2-{4-[2-(6-benzyloxy-pyridazin-3-yl)-ethyl]-phenoxy}-ethyl ester

Prepared analogously to IV.2.f from 2-{4-[2-(6-benzyloxy-pyridazin-3-yl)-ethyl]-phenoxy}-ethanol and methanesulfonyl chloride.

Yield: 0.7 g (95% of theory),

C₂₂H₂₄N₂O₅S

EII mass spectrum: m/z=429 [M+H]⁺

### Example IV.4

### Methanesulfonic acid 2-{6-[2-(6-benzyloxy-pyridazin-3-yl)-ethyl]-pyridin-3-yloxy}-ethyl ester

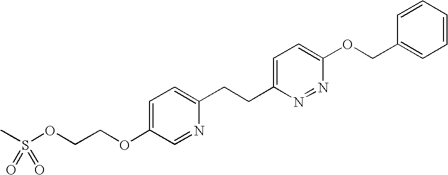

### IV.4.a

### 2-(6-Bromo-pyridin-3-yloxy)-ethanol

To a solution of 14 g (80.46 mmol) 2-Bromo-5-hydroxy-pyridine in 350 ml acetonitrile are added 21.57 ml (321.84 mmol) 2-Chloroethanol and 27.82 g (201.28 mmol) potassium carbonate. The mixture is refluxed for 18 hours and filtered. The filtrate is evaporated. Purification is achieved by chromatography (silica gel, ethyl acetate).

Yield: 17.3 g (99% of theory),

R_{f} value: 0.49 (silica gel, ethyl acetate)

### IV.4.b

### 2-[6-(6-Benzyloxy-pyridazin-3-ylethynyl)-pyridin-3-yloxy]-ethanol

Prepared analogously to III.1.c from 2-(6-Bromo-pyridin-3-yloxy)-ethanol and 3-benzyloxy-6-ethynyl-pyridazine.

Yield: 4.76 g (37% of theory),

C₂₀H₁₇N₃^{O}₃

EII mass spectrum: m/z=348 [M+H]⁺

### IV.4.c

### 2-{6-[2-(6-Benzyloxy-pyridazin-3-yl)-ethyl]-pyridin-3-yloxy}-ethanol

Prepared analogously to 1.1.d from 2-[6-(6-Benzyloxy-pyridazin-3-ylethynyl)-pyridin-3-yloxy]-ethanol.

Yield: 1.56 g (32% of theory),

R_{f} value: 0.3 (silica gel, methylene chloride/methanol/ammonia solution=90:10:1)

C₂₁H₂₂N₂O₃

EII mass spectrum: m/z=352 [M+H]⁺

### IV.4.d

### Methanesulfonic acid 2-{6-[2-(6-benzyloxy-pyridazin-3-yl)-ethyl]-pyridin-3-yloxy}-ethyl ester

Prepared analogously to IV.2.f from 2-{6-[2-(6-Benzyloxy-pyridazin-3-yl)-ethyl]-pyridin-3-yloxy}-ethanol I and methanesulfonyl chloride.

Yield: 1.82 g (95% of theory),

### Example IV.5

### Methanesulfonic acid 2-{3-[2-(6-benzyloxy-pyridazin-3-yl)-ethyl]-phenoxy}-ethyl ester

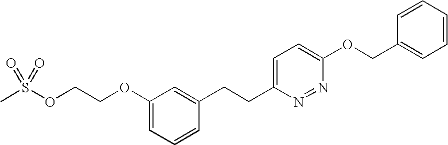

### IV.5.a

### 2-[3-(6-Benzyloxy-pyridazin-3-ylethynyl)-phenoxy]-ethanol

Prepared analogously to III.1.c from 3-benzyloxy-6-ethynyl-pyridazine and 2-(3-Iodo-phenoxy)-ethanol.

Yield: 6.67 g (52% of theory),

R_{f} value: 0.52 (silica gel, methylene chloride/methanol/ammonia solution=90:10:1)

C₂₁H₁₈N₂O₃

EII mass spectrum: m/z=347 [M+H]⁺

### IV.5.b

### 2-{3-[2-(6-Benzyloxy-pyridazin-3-yl)-ethyl]-phenoxy}-ethanol

Prepared analogously to 1.1.d from 2-[3-(6-Benzyloxy-pyridazin-3-ylethynyl)-phenoxy]-ethanol.

Yield: 4.88 g (79% of theory),

R_{f} value: 0.48 (silica gel, methylene chloride/methanol/ammonia solution=90:10:1)

C₂₁H₂₂N₂O₃

EII mass spectrum: m/z=351 [M+H]⁺

### IV.5.c.

### Methanesulfonic acid 2-{3-[2-(6-benzyloxy-pyridazin-3-yl)-ethyl]-phenoxy}-ethyl ester

Prepared analogously to IV.2.f from 2-{3-[2-(6-Benzyloxy-pyridazin-3-yl)-ethyl]-phenoxy}-ethanol and methanesulfonyl chloride.

Yield: 5.28 g (94% of theory),

R_{f} value: 0.64 (silica gel, methylene chloride/methanol/ammonia solution=90:10:1)

C₂₂H₂₄N₂O₅S

EII mass spectrum: m/z=429 [M+H]⁺

### Example V.1

### 3-(4-Fluoro-benzyloxy)-6-[4-(4-methoxy-piperidin-1-ylmethyl)-phenylethynyl]-pyridazine

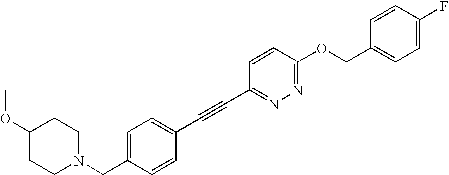

### V.1.a 3-(4-Fluoro-benzyloxy)-6-iodo-pyridazine

Prepared analogously to example 1.1.b from 3,6-diiodopyridazine and (4-fluoro-phenyl)-methanol.

Yield: 29 g (88% of theory),

retention time (HPLC): 3.18 min (method A)

M.p. 104-106° C.

C₁₁H₈F₁N₂O

EII mass spectrum: m/z=331 [M+H]⁺

### V.1.b 4-[4-(4-Fluoro-benzyloxy)-phenylethynyl]-benzaldehyde

Prepared analogously to example 1.1.c from 3-(4-fluoro-benzyloxy)-6-iodo-pyridazine and 4-ethynyl-benzaldehyde.

Yield: 1.4 g (100% of theory),

C₂₀H₁₃FN₂O₂

EII mass spectrum: m/z=333 [M+H]⁺

### V.1.c

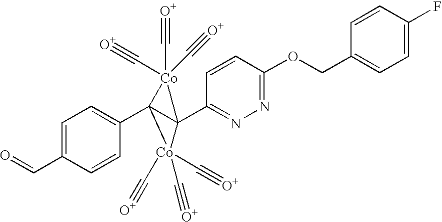

0.38 g (1 mmol) dicobald octacarbonyl are added to a suspension of 0.3 g (0.9 mmol) 4-[4-(4-fluoro-benzyloxy)-phenylethynyl]-benzaldehyde in 5 ml toluene. The reaction mixture is stirred for 20 hours at room temperature. The reaction mixture is concentrated. Purification is achieved by silica gel column chromatography with cyclohexane/EtOAc as eluent.

Yield: 1.4 g (100% of theory),

C₂₆H₁₃Co₂FN₂O₈

EII mass spectrum: m/z=619 [M+H]⁺

### V.1.d

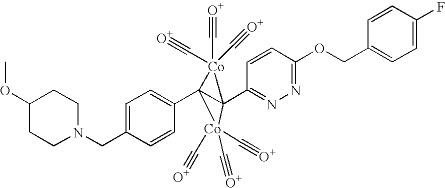

Prepared analogously to example 1.1 from cobald compound V.1.c and 4-methoxy-piperidine.

Yield: 0.36 g (83% of theory),

R_{f} value: 0.45 (silica gel, methylene chloride/ethanol=20:1)

C₃₂H₂₆CO₂FN₃O₈

EII mass spectrum: m/z=718 [M+H]⁺

### V.1.e 3-(4-Fluoro-benzyloxy)-6-[4-(4-methoxy-piperidin-1-ylmethyl)-phenylethynyl]-pyridazine

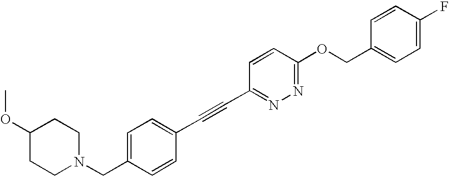

1.096 g (2 mmol) ammonium cerium(IV) nitrate are added to a mixture of 0.359 g 0.5 mmol) of cobald compound V.1.d and 5 ml methanol. The reaction mixture is stirred for 30 minutes at room temperature. Saturated sodium chloride solution is added and the mixture is extracted with EtOAc. The organic phase is dried over sodium sulphate and concentrated. Purification is achieved by silica gel column chromatography with methylene chloride/methanol as eluent.

Yield: 0.36 g (83% of theory),

R_{f} value: 0.4 (silica gel, methylene chloride/ethanol=20:1)

C₂₆H₂₆FN₃O₂

EII mass spectrum: m/z=432 [M+H]⁺

### Example V.2

### [5-(6-Benzyloxy-pyridazin-3-ylethynyl)-pyridin-2-yl]-(2-pyrrolidin-1-yl-ethyl)-amine

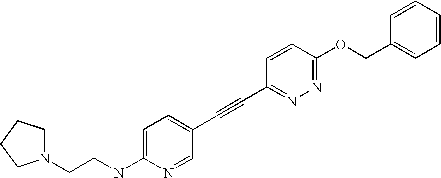

### V.2.a (5-Bromo-pyridin-2-yl)-(2-pyrrolidin-1-yl-ethyl)-amine

A mixture of 4.88 g (20 mmol) 2,5-dibromo-pyridine and 5.172 ml (40 mmol) 1-(2-aminoethyl)-pyrrolidine is stirred for 20 minutes at 100° C. 100 ml EtOAc is added and the mixture is extracted with 100 ml water. The organic phase is dried of sodium sulphate. Purification is achieved by silica gel column chromatography with EtOAc/methanol/ammonia solution as eluent.

Yield: 2.15 g (40% of theory),

R_{f} value: 0.44 (silica gel, EtOAc/methanol/ammonia solution=90:10:1)

C₁₁H₁₆BrN₃

EII mass spectrum: m/z=270/272 [M+H]⁺

### V.2.b (5-Iodo-pyridin-2-yl)-(2-pyrrolidin-1-yl-ethyl)-amine

37 mg (0.19 mmol) copper(I) iodide are added to a mixture of 500 mg (2 mmol) (5-bromo-pyridin-2-yl)-(2-pyrrolidin-1-yl-ethyl)-amine and dioxin under nitrogen. Then 41 μl (0.39 mmol)

N,N′-dimethylethylenediamine and 0.585 g (3.9 mmol) sodium iodide are added under nitrogen. The reaction mixture is refluxed for 18 hours. A solution of ammonia (30% in water) is added. The resulting mixture is extracted with EtOAc. The organic phase is dried over sodium sulphate and concentrated.

Yield: 29 g (88% of theory),

retention time (HPLC): 1.75 min (method A)

C₁₁H₁₆IN₃

EII mass spectrum: m/z=318 [M+H]⁺

### V.2.c (2-Pyrrolidin-1-yl-ethyl)-(5-trimethylsilanylethynyl-pyridin-2-yl)-amine

Prepared analogously to example IV.2.b from (5-iodo-pyridin-2-yl)-(2-pyrrolidin-1-yl-ethyl)-amine and ethynyl-trimethyl-silane.

Yield: 310 mg (67% of theory),

retention time (HPLC): 2.66 min (method A)

C₁₆H₂₅N₃Si

EII mass spectrum: m/z=288 [M+H]⁺

### V.2.d (5-Ethynyl-pyridin-2-yl)-(2-pyrrolidin-1-yl-ethyl)-amine

2.43 ml (2.43 mmol) of 1M sodium hydroxide solution are added to a solution of 310 mg (1.07 mmol) (2-pyrrolidin-1-yl-ethyl)-(5-trimethylsilanylethynyl-pyridin-2-yl)-amine in 15 ml methanol. The reaction mixture is stirred for 18 hours and concentrated. Water is added and the mixture is extracted two times with EtOAc. The combined organic phases are dried over sodium sulphate and concentrated.

Yield: 130 mg (56% of theory),

retention time (HPLC): 1.55 min (method A)

C₁₃H₁₇N₃

EII mass spectrum: m/z=216 [M+H]⁺

### V.2.e [5-(6-Benzyloxy-pyridazin-3-ylethynyl)-pyridin-2-yl]-(2-pyrrolidin-1-yl-ethyl)-amine

Prepared analogously to example 1.1.c from (5-ethynyl-pyridin-2-yl)-(2-pyrrolidin-1-yl-ethyl)-amine and 3-benzyloxy-6-iodo-pyridazine.

Yield: 110 mg (45% of theory),

retention time (HPLC): 2.58 min (method A)

C₂₄H₂₅N₅O

EII mass spectrum: m/z=400 [M+H]⁺

### Example V.3

### 3-Benzyloxy-6-[6-(2-pyrrolidin-1-yl-ethoxy)-pyridin-3-ylethynyl]-pyridazine trifluoro acetate

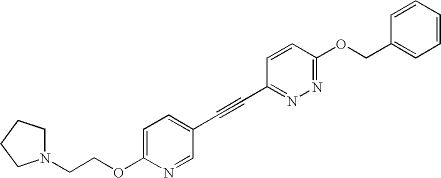

### V.3.a 5-Bromo-2-(2-pyrrolidin-1-yl-ethoxy)-pyridine

442 mg (10.13 mmol) of sodium hydride (55%) are added to a solution of 0.987 ml (8.44 mmol) 2-pyrrolidin-1-yl-ethanol in 30 ml dry DMF. The mixture is stirred for 30 minutes at room temperature. 2 g (8.44 mmol) 2,5-dibromo-pyridine are added and the reaction mixture is stirred at 80° C. for five hours. After that time the reaction mixture is poured in water and extracted with EtOAc. The organic phase is dried over sodium sulphate and concentrated.

Yield: 1.8 g (78% of theory),

retention time (HPLC): 1.4 min (method A)

C₁₁H₁₅BrN₂O

EII mass spectrum: m/z=270/272 [M+H]⁺

### V.3.b 5-Iodo-2-(2-pyrrolidin-1-yl-ethoxy)-pyridine

Prepared analogously to example V.2.b from 5-bromo-2-(2-pyrrolidin-1-yl-ethoxy)-pyridine.

Yield: 1.9 g (60% of theory),

retention time (HPLC): 1.75 min (method A)

C₁₁H₁₅IN₂O

EII mass spectrum: m/z=319 [M+H]⁺

### V.3.c 3-Benzyloxy-6-[6-(2-pyrrolidin-1-yl-ethoxy)-pyridin-3-ylethynyl]-pyridazine trifluoro acetate

Prepared analogously to example 1.1.c from 5-iodo-2-(2-pyrrolidin-1-yl-ethoxy)-pyridine and 3-benzyloxy-6-ethynyl-pyridazine.

Yield: 280 mg (89% of theory),

retention time (HPLC): 2.25 min (method A)

C₂₄H₂₄N₄O₂.C₂HF₃O₂

EII mass spectrum: m/z=401 [M+H]⁺

### Example V.4

### 1-{7-[2-(6-Benzyloxy-pyridazin-3-yl)-ethyl]-1,2,4,5-tetrahydro-benzo[d]azepin-3-yl}-2,2,2-trifluoro-ethanone

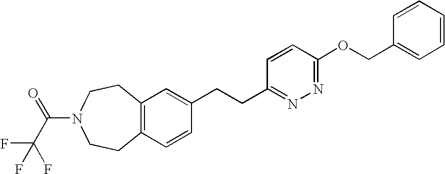

### V.4.a 2,2,2-Trifluoro-1-(7-iodo-1,2,4,5-tetrahydro-benzo[d]azepin-3-yl)-ethanone

3.09 g (12 mmol) 1-(7-amino-1,2,4,5-tetrahydro-benzo[d]azepin-3-yl)-2,2,2-trifluoro-ethanone are added to a mixture of 36 ml water and 3.6 ml concentrated sulphuric acid. At 0° C. a solution of 910 mg (13.2 mmol) sodium nitrit in 18 ml water is added. After ten minutes a mixture of 2.988 g (18 mmol) sodium iodide in 3.6 ml 1N sulphuric acid is added. The reaction mixture is stirred for 18 hours at room temperature. The reaction mixture is extracted with a mixture of methylene chloride and sodium disulfite solution. The organic phase is dried and concentrated. Purification is achieved by silica gel column chromatography with methylene chloride as eluent.

Yield: 3.1 g (70% of theory),

R_{f} value: 0.75 (silica gel, methylene chloride)

### V.4.b 1-[7-(6-Benzyloxy-pyridazin-3-ylethynyl)-1,2,4,5-tetrahydro-benzo[d]azepin-3-yl]-2,2,2-trifluoro-ethanone

Prepared analogously to example 1.1.c from 2,2,2-trifluoro-1-(7-iodo-1,2,4,5-tetrahydro-benzo[d]azepin-3-yl)-ethanone and 3-benzyloxy-6-ethynyl-pyridazine.

Yield: 640 mg (44% of theory),

retention time (HPLC): 3.6 min (method A)

C₂₅H₂₀F₃N₃O₂

EII mass spectrum: m/z=452 [M+H]⁺

### V.4.c 1-{7-[2-(6-Benzyloxy-pyridazin-3-yl)-ethyl]-1,2,4,5-tetrahydro-benzo[d]azepin-3-yl}-2,2,2-trifluoro-ethanone

Prepared analogously to example 1.1.d from 1-[7-(6-benzyloxy-pyridazin-3-ylethynyl)-1,2,4,5-tetrahydro-benzo[d]azepin-3-yl]-2,2,2-trifluoro-ethanone.

Yield: 427 mg (68% of theory),

retention time (HPLC): 3.45 min (method A)

C₂₅H₂₄F₃N₃O₂

EII mass spectrum: m/z=456 [M+H]⁺

### Example V.5

### 7-[2-(6-Benzyloxy-pyridazin-3-yl)-ethyl]-3,4-dihydro-1H-isoquinoline-2-carboxylic acid tert-butyl ester

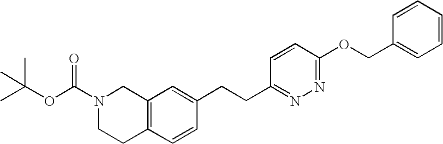

### V.5.a 7-Iodo-3,4-dihydro-1H-isoquinoline-2-carboxylic acid tert-butyl ester

To a mixture of 55.8 g (0.179 mol) 7-Bromo-3,4-dihydro-1H-isoquinoline-2-carboxylic acid tert-butyl ester in 180 ml Dioxan are added 3.473 g (17.87 mmol) copper (I) iodide, 53.58 g (357.41 mmol) sodium iodide and 3.806 ml (35.74 mmol) N,N′-Dimethylethylenediamine. The reaction mixture is refluxed for 18 hours. 300 ml of 5% aqueous ammonia solution are added and the mixture is extracted two times with ethyl acetate. The combined organic extracts are extracted with aqueous ammonia solution and then water. The organic phases are dried over magnesium sulphate and concentrated to dryness. The residue is washed with petrol ether.

Yield: 35.4 g (55% of theory),

C₁₄H₁₈INO₂

EII mass spectrum: m/z=360 [M+H]⁺

### V.5.b 7-(6-Benzyloxy-pyridazin-3-ylethynyl)-3,4-dihydro-1H-isoquinoline-2-carboxylic acid tert-butyl ester

Prepared analogously to example III.1.c from 3-Benzyloxy-6-ethynyl-pyridazine and 7-Iodo-3,4-dihydro-1H-isoquinoline-2-carboxylic acid tert-butyl ester.

Yield: 2.33 g (55% of theory),

### V.5.c 7-[2-(6-Benzyloxy-pyridazin-3-yl)-ethyl]-3,4-dihydro-1H-isoquinoline-2-carboxylic acid tert-butyl ester

Prepared analogously to example III.1.d from 7-(6-Benzyloxy-pyridazin-3-ylethynyl)-3,4-dihydro-1H-isoquinoline-2-carboxylic acid tert-butyl ester.

Yield: 1.2 g (91% of theory),

retention time (HPLC): 3.603 min (method A)

C₂₇H₃₁N₃O₃

EII mass spectrum: m/z=446 [M+H]⁺

### Example V.6

### 6-(6-Benzyloxy-pyridazin-3-ylethynyl)-1,2,3,4-tetrahydro-isoquinoline trifluoroacetate

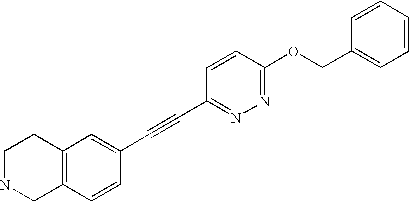

### V.6.a 6-Bromo-3,4-dihydro-1H-isoquinoline-2-carboxylic acid tert-butyl ester

Prepared analogously to example V.5.a from 6-Iodo-3,4-dihydro-1H-isoquinoline-2-carboxylic acid tert-butyl ester.

Yield: 14 g (94% of theory),

C₁₄H₁₈INO₂

EII mass spectrum: m/z=359 [M]⁺

### V.6.b 6-Trimethylsilanylethynyl-3,4-dihydro-1H-isoquinoline-2-carboxylic acid tert-butyl ester

Prepared analogously to example III.1.a from 6-Bromo-3,4-dihydro-1H-isoquinoline-2-carboxylic acid tert-butyl ester and Ethynyl-trimethyl-silane.

Yield: 2.9 g

R_{f} value: 0.73 (silica gel, petrol ether/ethyl acetate=1:1)

### V.6.c 6-Ethynyl-3,4-dihydro-1H-isoquinoline-2-carboxylic acid tert-butyl ester

Prepared analogously to example III.1.b from 6-Trimethylsilanylethynyl-3,4-dihydro-1H-isoquinoline-2-carboxylic acid tert-butyl ester.

Yield: 0.79 g (42% of theory),

R_{f} value: 0.86 (silica gel, petrol ether/ethyl acetate=1:1)

### V.6.d 6-(6-Benzyloxy-pyridazin-3-ylethynyl)-3,4-dihydro-1H-isoquinoline-2-carboxylic acid tert-butyl ester

Prepared analogously to example III.1.c from 6-Ethynyl-3,4-dihydro-1H-isoquinoline-2-carboxylic acid tert-butyl ester and 3-Benzyloxy-6-iodo-pyridazine.

Yield: 0.62 g (45% of theory),

R_{f} value: 0.7 (silica gel, methyle chloride/methanol/ammonia=90:10:1)

C₂₇H₂₇N₃O₃

EII mass spectrum: m/z=442 [M]⁺

### V.6.e 6-(6-Benzyloxy-pyridazin-3-ylethynyl)-1,2,3,4-tetrahydro-isoquinoline trifluoroacetate

Prepared analogously to example 27.1 (end compounds) from 6-(6-Benzyloxy-pyridazin-3-ylethynyl)-3,4-dihydro-1H-isoquinoline-2-carboxylic acid tert-butyl ester.

Yield: 0.6 g (94% of theory),

R_{f} value: 0.7 (silica gel, methylene chloride/methanol/ammonia=90:10:1)

C₂₂H₁₉N₃O.C₂HF₃O₂

EII mass spectrum: m/z=342 [M]⁺

### Example V.7

### 1-[4-(6-Benzyloxy-pyridazin-3-ylethynyl)-benzyl]-4-methyl-piperidin-4-ol

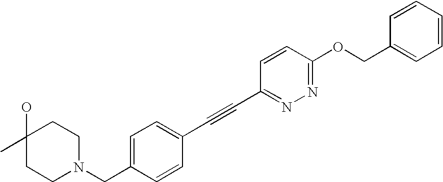

### V.7.a [4-(6-Benzyloxy-pyridazin-3-ylethynyl)-phenyl]-methanol

Prepared analogously to example III.1.c from (4-Ethynyl-phenyl)-methanol and 3-Benzyloxy-6-iodo-pyridazine.

Yield: 4.8 g (90% of theory),

C₂₀H₁₆N₂O₂

EII mass spectrum: m/z=317 [M]⁺

### V.7.b Methanesulfonic acid 4-(6-benzyloxy-pyridazin-3-ylethynyl)-benzyl ester

Prepared analogously to example IV.2.f from [4-(6-Benzyloxy-pyridazin-3-ylethynyl)-phenyl]-methanol and methane sulfonyl chloride.

Yield: 3 g (100% of theory)

C₂₁H₁₈N₂O₄S

EII mass spectrum: m/z=395 [M]⁺

### V.7.c 1-[4-(6-Benzyloxy-pyridazin-3-ylethynyl)-benzyl]-4-methyl-piperidin-4-ol

Prepared analogously to example 35.1 from Methanesulfonic acid 4-(6-benzyloxy-pyridazin-3-ylethynyl)-benzyl ester and 4-Methyl-piperidin-4-ol.

Yield: 0.24 g (76% of theory)

C₂₆H₂₇N₃O₂

EII mass spectrum: m/z=414 [M]⁺

### Example V.8

### 3-[2-(6-Benzyloxy-pyridazin-3-yl)-ethyl]-7,8-dihydro-5H-[1,6]naphthyridine-6-carboxylic acid tert-butyl ester

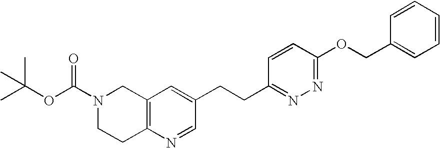

### V.8.a 3-Iodo-7,8-dihydro-5H-[1,6]naphthyridine-6-carboxylic acid tert-butyl ester

To a solution of 8 g (32.08 mmol) 3-Amino-7,8-dihydro-5H-[1,6]naphthyridine-6-carboxylic acid tert-butyl ester in 60 ml carbon tetrachloride are added 12.72 ml tert-Butyl nitrite (96.26 mmol) and 16.288 g (64.16 mmol) iodine. The mixture is stirred in the dark for 12 hours. The solvent is evaporated and methylene chloride is added. The mixture is extracted with water. The organic phase is dried over magnesium sulphate. Purification is achieved by chromatography (silica gel, petro ether/ethyl acetate=85: 15-7:3).

Yield: 0.9 g (8% of theory),

R_{f} value: 0.37 (silica gel, petrol ether/ethylacetate=7:3)

C₁₃H₁₇IN₂O₂

EII mass spectrum: m/z=361 [M]⁺

### V.8.b 3-Trimethylsilanylethynyl-7,8-dihydro-5H-[1,6]naphthyridine-6-carboxylic acid tert-butyl ester

Prepared analogously to example III.1.a from 3-Iodo-7,8-dihydro-5H-[1,6]naphthyridine-6-carboxylic acid tert-butyl ester and ethynyl-trimethyl-silane.

Yield: 0.63 g (76% of theory)

R_{f} value: 0.41 (silica gel, petrol ether/ethylacetate=7:3)

C₁₈H₂₆N₂O₂Si

EII mass spectrum: m/z=331 [M]⁺

### V.8.c 3-Ethynyl-7,8-dihydro-5H-[1,6]naphthyridine-6-carboxylic acid tert-butyl ester

A mixture of 630 mg (1.9 mmol) and 548 mg (2.09 mmol) Tetrabutyl ammonium fluoride in 10 ml of methylene chloride is stirred for 18 hours at room temperature. Purification is achieved by chromatography (silica gel, cyclohexane/ethyl acetate=1:1).

Yield: 360 mg (73% of theory)

R_{f} value: 0.36 (silica gel, cyclohexane/ethyl acetate=1:1)

C₁₅H₁₈N₂O₂

EII mass spectrum: m/z=259 [M]⁺

### V.8.d 3-(6-Benzyloxy-pyridazin-3-ylethynyl)-7,8-dihydro-5H-[1,6]naphthyridine-6-carboxylic acid tert-butyl ester

Prepared analogously to example III.1.c from 3-Ethynyl-7,8-dihydro-5H-[1,6]naphthyridine-6-carboxylic acid tert-butyl ester and 3-benzyloxy-6-iodo-pyridazine.

Yield: 420 mg (68% of theory)

C₂₆H₂₆N₄O₃

EII mass spectrum: m/z=443 [M]⁺

### V.8.e 3-[2-(6-Benzyloxy-pyridazin-3-yl)-ethyl]-7,8-dihydro-5H-[1,6]naphthyridine-6-carboxylic acid tert-butyl ester

Prepared analogously to example III.1.d from 3-(6-Benzyloxy-pyridazin-3-ylethynyl)-7,8-dihydro-5H-[1,6]naphthyridine-6-carboxylic acid tert-butyl ester.

Yield: 320 mg (75% of theory)

C₂₆H₃₀N₄O₃

EII mass spectrum: m/z=447 [M]⁺

### Preparation of the End Compounds

### Example 1.1

### 4-{4-[2-(6-Benzyloxy-pyridazin-3-yl)-ethyl]benzyl}-morpholine

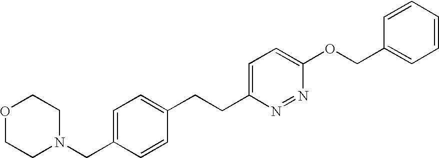

A mixture of 50 mg (0.157 mmol) 4-(6-benzyloxy-pyridazin-3-ylethyl)-benzaldehyde (example I.1) 0.014 ml (0.16 mmol) morpholine, 0.406 g (0.84 mmol) MP-triacetoxy-borohydride (2.07 mmol/g, Argonaut) and 0.024 ml (0.4 mmol) of glacialic acid in 5 ml of THF is stirred for 24 hours. The mixture is filtered and the filtrate is concentrated. The residue is purified by HPLC (method 2). The purified product is dissolved in methylene chloride and passed through a column (StratoSpheres SPE PL-HCO3 MP Resin) and concentrated.

Yield: 26 mg (42.5% of theory),

retention time (HPLC): 2.37 min (method A)

C₂₄H₂₇N₃O₂

EII mass spectrum: m/z=390 [M+H]⁺

### Example 2

The following compounds of general formula I are prepared analogously to Example 1.1, the educts used being shown in the column headed “Educts”:

(I)

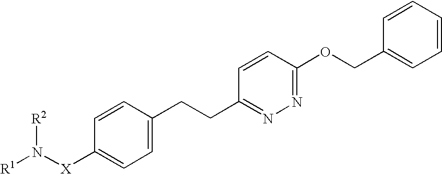

| Example | R¹R²N—X— | Educts | mass spectrum | Retention time (HPLC) |
|---|---|---|---|---|
| 2.1 | 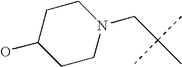 | I.1 | 404 [M + H]⁺ | 2.42 (A) |
| 2.2 | 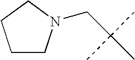 | I.1 | 374 [M + H]⁺ | 2.45 (A) |
| 2.3 | 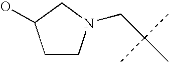 | I.1 | 390 [M + H]⁺ | 2.46 (A) |
| 2.4 | 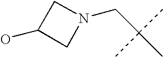 | I.1 | 376 [M + H]⁺ | 2.44 (A) |
| 2.5 | 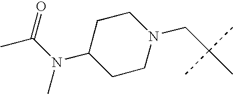 | I.1 II.2 | 459 [M + H]⁺ | 2.49 (A) |
| 2.6 | 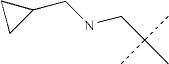 | I.1 | 374 [M + H]⁺ | 2.53 (A) |
| 2.7 | 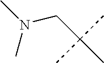 | I.1 | 348 [M + H]⁺ | 2.34 (A) |
| 2.8 | 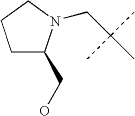 | I.1 | 404 [M + H]⁺ | 2.44 (A) |
| 2.9 | 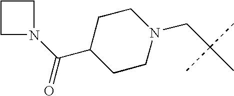 | I.1 II.1 | 471 [M + H]⁺ | 2.47 (A) |
| 2.10 | 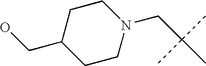 | I.1 | 418 [M + H]⁺ | 2.4 (A) |
| 2.11 | 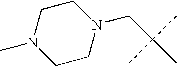 | I.1 | 403 [M + H]⁺ | 2.32 (A) |
| 2.12 | 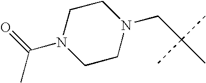 | I.1 | 431 [M + H]⁺ | 2.41 (A) |
| 2.13 | 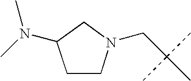 | I.1 | 417 [M + H]⁺ | 2.09 (A) |
| 2.14 | 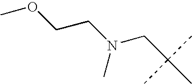 | I.1 | 392 [M + H]⁺ | 2.4 (A) |
| 2.15 | 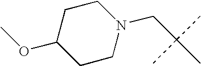 | I.1 | 418 [M + H]⁺ | 2.51 (A) |
| 2.16 | 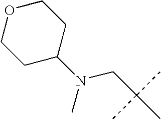 | I.1 | 418 [M + H]⁺ | 2.42 (A) |
| 2.17 | 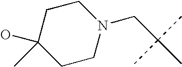 | I.1 | 418 [M + H]⁺ | 2.42 (A) |
| 2.18 | 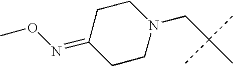 | I.1 II.3 | 431 [M + H]⁺ | 2.52 (A) |
| 2.19 | 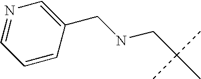 | I.1 | 411 [M + H]⁺ | 2.18 (A) |
| 2.20 | 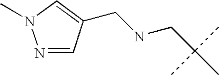 | I.1 | 414 [M + H]⁺ | 2.27 (A) |
| 2.21 | 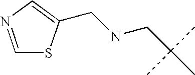 | I.1 | 417 [M + H]⁺ | 2.35 (A) |
| 2.22 | 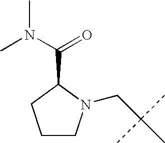 | I.1 | 445 [M + H]⁺ | 2.27 (A) |
| 2.23 | 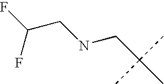 | I.1 | 384 [M + H]⁺ | 2.32 (A) |
| 2.24 | 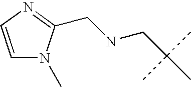 | I.1 | 414 [M + H]⁺ | 2.16 (A) |
| 2.25 | 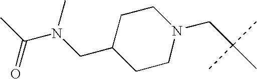 | I.1 und II.4 | 473 [M + H]⁺ | 2.26 (A) |
| 2.26 | 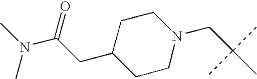 | I.1 und II.5 | 473 [M + H]⁺ | 2.50 (A) |
| 2.27 | 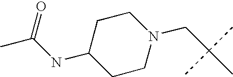 | I.1 | 445 [M + H]⁺ | 2.51 (A) |
| 2.28 | 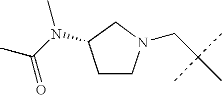 | I.1 | 445 [M + H]⁺ | 2.48 (A) |
| 2.29 | 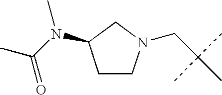 | I.1 | 445 [M + H]⁺ | 2.50 (A) |
| 2.30 | 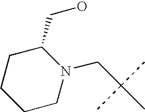 | I.1 | 418 [M + H]⁺ | 2.50 (A) |
| 2.31 | 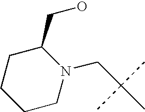 | I.1 | 418 [M + H]⁺ | 2.61 (A) |
| 2.32 | 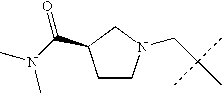 | I.1 and II.6 | 445 [M + H]⁺ | 2.51 (A) |
| 2.33 | 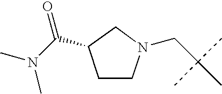 | I.1 and II.7 | 445 [M + H]⁺ | 2.50 (A) |
| 2.34 | 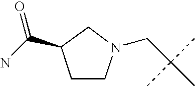 | I.1 | 417 [M + H]⁺ | 2.48 (A) |
| 2.35 | 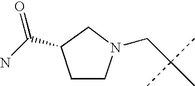 | I.1 | 417 [M + H]⁺ | 2.66 (A) |
| 2.36 | 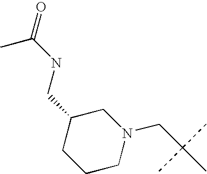 | I.1 and II.8 | 459 [M + H]⁺ | 2.28 (A) |
| 2.37 | 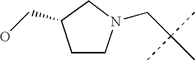 | I.1 | 404 [M + H]⁺ | 2.51 (A) |
| 2.38 | 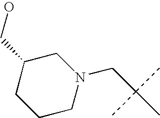 | I.1 | 418 [M + H]⁺ | 2.26 (A) |
| 2.39 | 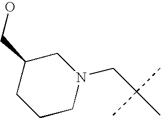 | I.1 | 418 [M + H]⁺ | 2.26 (A) |
| 2.40 | 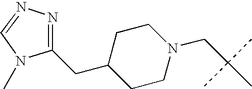 | I.1 | 469 [M + H]⁺ | 2.08 (A) |

### Example 3.1

### 1-{6-[2-(6-Benzyloxy-pyridazin-3-yl)-ethyl]-pyridin-3-ylmethyl}-piperidin-4-ol

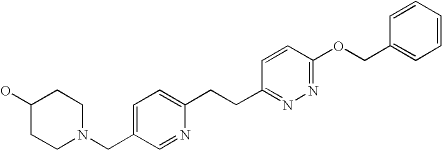

A mixture of 167 mg (0.47 mmol) of 6-[2-(6-benzyloxy-pyridazin-3-yl)-ethyl]-pyridine-3-carbaldehyde (example III.1), 48 mg (0.47 mmol) of 4-hydroxy-piperidine, 56 μl (1.2 mmol) of glacialic acid and 1.14 g (2.35 mmol) of MP-triacetoxyborohydride (2.07 mmol/g, Argonaut) and in 7 ml of dry THF is placed in a shaker for 48 hours at room temperature. The mixture is filtered and the filtrate is concentrated. Purification of the residue is achieved by silica gel column chromatography with methylene chloride/MeOH/ammonia solution as eluent.

Yield: 30 mg (23% of theory),

retention time (HPLC): 2.02 min (method A)

C₂₄H₂₈N₄^{O}₂

EII mass spectrum: m/z=405 [M+H]⁺

### Example 3.2

### 3-Benzyloxy-6-{2-[5-(2-methyl-pyrrolidin-1-ylmethyl)-pyridin-2-yl]-ethyl}-pyridazine

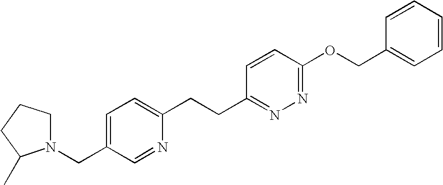

A mixture of 40 mg (0.12 mmol) of 3-Benzyloxy-6-[2-(5-chloromethyl-pyridin-2-yl)-ethyl]-pyridazine (example III.2), 12 mg (0.14 mmol) of 2-methyl-pyrrolidine and 36 mg (0.26 mmol) potassium carbonate in 6 ml of acetone and a few drops of water is refluxed for 24 hours. The mixture is concentrated. Methylene chloride is added to the residue and the mixture is extracted with water. The organic phase is collected by passing the reaction mixture through a column (Phase Separator/Separtis) and concentrated. The residue is purified by HPLC (method 3). The purified product is dissolved in methylene chloride and passed through a column (StratoSpheres SPE PL-HCO3 MP Resin) and concentrated.

Yield: 12 mg (26% of theory),

retention time (HPLC): 2.2 min (method A)

C₂₄H₂₈N₄O

EII mass spectrum: m/z=389 [M+H]⁺

### Example 4

The following compounds of general formula II are prepared analogously to Example 3.1 or 3.2, the educts used being shown in the column headed “Educts”:

(II)

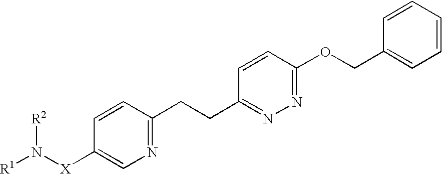

| Example | R¹R²N—X— | Educts | mass spectrum | Retention time (HPLC) |
|---|---|---|---|---|
| 4.1 | 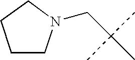 | III.1 | 375 [M + H]⁺ | 2.09 (A) |
| 4.2 | 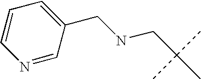 | III.1 | 412 [M + H]⁺ | 1.96 (A) |
| 4.3 | 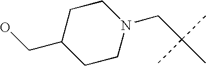 | III.1 | 419 [M + H]⁺ | 2.02 (A) |
| 4.4 | 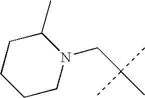 | III.2 | 403 [M + H]⁺ | 2.26 (A) |
| 4.5 | 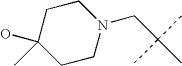 | III.1 | 419 [M + H]⁺ | 2.17 (A) |
| 4.6 | 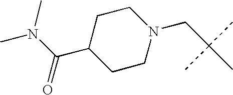 | III.1 | 460 [M + H]⁺ | 2.21 (A) |
| 4.7 | 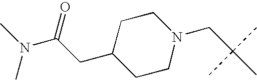 | III.1 and III.5 | 474 [M + H]⁺ | 2.22 (A) |
| 4.8 | 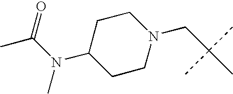 | III.1 and II.2 | 460 [M + H]⁺ | 2.23 (A) |
| 4.9 | 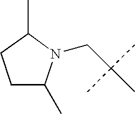 | III.1 | 403 [M + H]⁺ | 2.08 (A) |
| 4.10 | 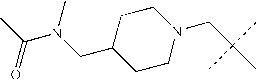 | III.1 and II.4 | 474 [M + H]⁺ | 2.07 (A) |
| 4.11 | 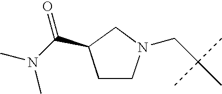 | III.1 und II.6 | 446 [M + H]⁺ | 2.05 (A) |
| 4.12 | 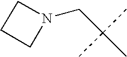 | III.2 | 361 [M + H]⁺ | 1.94 (A) |
| 4.13 | 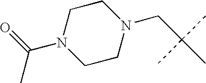 | III.2 | 432 [M + H]⁺ | 1.96 (A) |
| 4.14 | 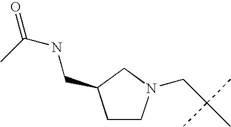 | III.2 | 446 [M + H]⁺ | 1.88 (A) |
| 4.15 | 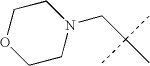 | III.2 | 391 [M + H]⁺ | 1.82 (A) |
| 4.16 | 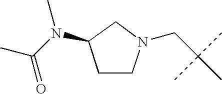 | III.2 | 446 [M + H]⁺ | 1.90 (A) |
| 4.17 | 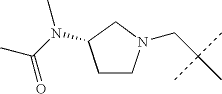 | III.2 | 446 [M + H]⁺ | 1.92 (A) |
| 4.18 | 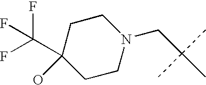 | III.2 | 473 [M + H]⁺ | 2.08 (A) |
| 4.19 | 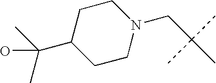 | III.2 | 447 [M + H]⁺ | 1.93 (A) |
| 4.20 | 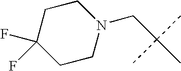 | III.2 | 425 [M + H]⁺ | 2.05 (A) |
| 4.21 | 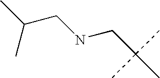 | III.2 | 377 [M + H]⁺ | 1.96 (A) |
| 4.22 | 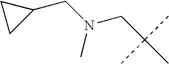 | III.2 | 389 [M + H]⁺ | 1.96 (A) |

The following compounds of general formula II can be prepared analogously to Example 3.1 or 3.2:

(II)

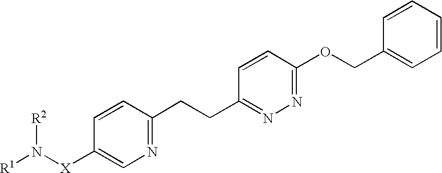

| Example | R¹R²N—X— |
|---|---|
| 4.23 | 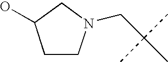 |
| 4.24 | 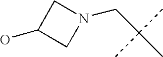 |
| 4.25 | 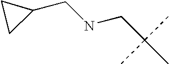 |
| 4.26 | 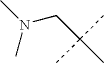 |
| 4.27 | 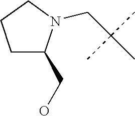 |
| 4.28 | 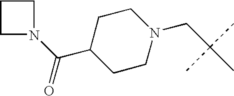 |
| 4.29 | 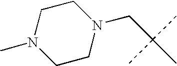 |
| 4.30 | 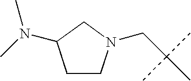 |
| 4.31 | 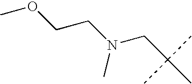 |
| 4.32 | 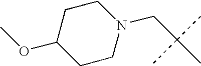 |
| 4.33 | 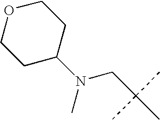 |
| 4.34 | 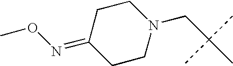 |
| 4.35 | 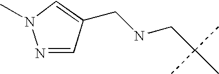 |
| 4.36 | 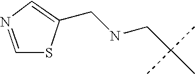 |
| 4.37 | 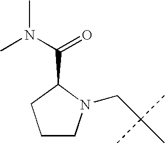 |
| 4.38 | 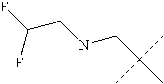 |
| 4.39 | 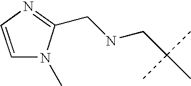 |
| 4.40 | 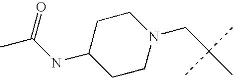 |
| 4.41 | 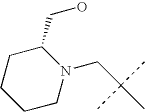 |
| 4.42 | 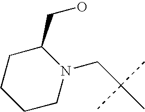 |
| 4.43 | 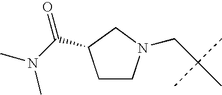 |
| 4.44 | 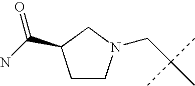 |
| 4.45 | 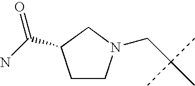 |
| 4.46 | 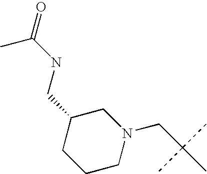 |
| 4.47 | 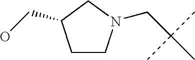 |
| 4.48 | 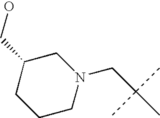 |
| 4.49 | 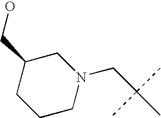 |
| 4.50 | 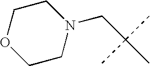 |
| 4.51 | 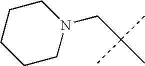 |

### Example 5.1

### 3-Phenoxy-6-{2-[4-(2-pyrrolidin-1-yl-ethoxy)-phenyl]-ethyl}-pyridazine

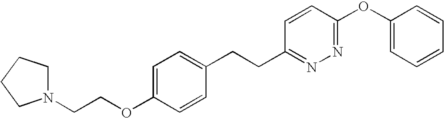

A mixture of 100 mg (0.24 mmol) of methanesulfonic acid 2-{4-[2-(6-phenoxy-pyridazin-3-yl)-ethyl]-phenoxy}-ethyl ester (example IV.2), 41 mg (0.3 mmol) potassium carbonate and 0.021 ml (0.25 mmol) pyrrolidine in 5 ml acetone and a few drops of water are refluxed for 48 hours. The reaction mixture is concentrated. The residue is extracted with methylene chloride and water. The organic phase is collected by passing the reaction mixture through a column (Phase Separator/Separtis) and concentrated. The residue is purified by HPLC (method 3). The purified product is dissolved in methylene chloride and passed through a column (StratoSpheres SPE PL-HCO3 MP Resin) and concentrated.

Yield: 62 mg (66% of theory),

retention time (HPLC): 2.27 min (method A)

C₂₄H₂₇N₃O₂

EII mass spectrum: m/z=390 [M+H]⁺

### Example 6

The following compounds of general formula III are prepared analogously to Example 5.1, the educts used being shown in the column headed “Educts”:

(III)

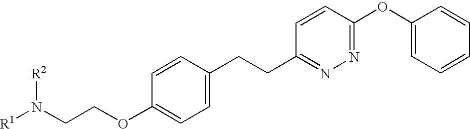

| Example | R¹R²N— | Educts | mass spectrum | Retention time (HPLC) |
|---|---|---|---|---|
| 6.1 | 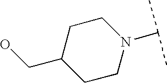 | IV.2 | 434 [M + H]⁺ | 2.2 (A) |
| 6.2 | 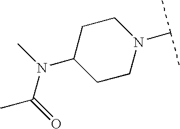 | IV.2 And II.2 | 475 [M + H]⁺ | 2.22 (A) |

### Example 7.1

### 3-Benzyloxy-6-{2-[4-(2-pyrrolidin-1-yl-ethoxy)-phenyl]-ethyl}-pyridazine

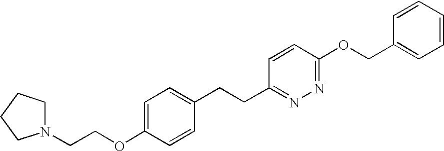

A mixture of 120 mg (0.28 mmol) of methanesulfonic acid 2-{4-[2-(6-benzyloxy-pyridazin-3-yl)-ethyl]-phenoxy}-ethyl ester (example IV.3), 41 mg (0.3 mmol) potassium carbonate and 0.023 ml (0.28 mmol) pyrrolidine in 5 ml acetone and a few drops of water are refluxed for 48 hours. The reaction mixture is concentrated. The residue is extracted with methylene chloride and water. The organic phase is collected by passing the reaction mixture through a column (Phase Separator/Separtis) and concentrated. The residue is purified by HPLC (method 3). The purified product is dissolved in methylene chloride and passed through a column (StratoSpheres SPE PL-HCO3 MP Resin) and concentrated.

Yield: 65 mg (57% of theory),

retention time (HPLC): 2.35 min (method A)

C₂₅H₂₉N₃O₂

EII mass spectrum: m/z=404 [M+H]⁺

### Example 8

The following compounds of general formula IV are prepared analogously to Example 7.1, the educts used being shown in the column headed “Educts”:

(IV)

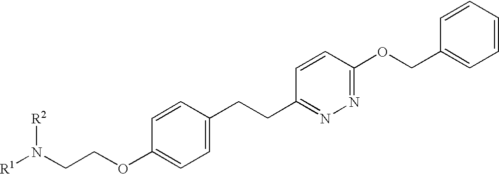

| Example | R¹R²N— | Educts | mass spectrum | Retention time (HPLC) |
|---|---|---|---|---|
| 8.1 | 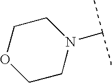 | IV.3 | 420 [M + H]⁺ | 2.42 (A) |
| 8.2 | 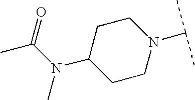 | IV.3 and II.2 | 489 [M + H]⁺ | 2.30 (A) |
| 8.3 | 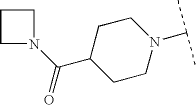 | IV.3 and II.1 | 501 [M + H]⁺ | 2.43 (A) |
| 8.4 | 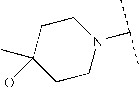 | IV.3 | 448 [M + H]⁺ | |
| 8.5 | 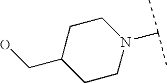 | IV.3 | 448 [M + H]⁺ | 2.23 (A) |
| 8.6 | 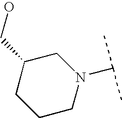 | IV.3 | 448 [M + H]⁺ | 2.30 (A) |
| 8.7 | 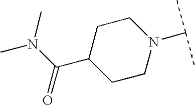 | IV.3 | 489 [M + H]⁺ | 2.32 (A) |
| 8.8 | 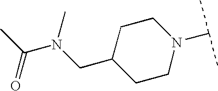 | IV.3 and II.5 | 503 [M + H]⁺ | 2.31 (A) |
| 8.9 | 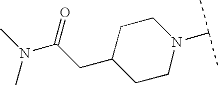 | IV.3 and II.5 | 503 [M + H]⁺ | 2.32 (A) |
| 8.10 | 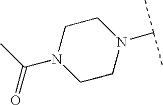 | IV.3 | 406 [M + H]⁺ | 2.29 (A) |
| 8.11 | 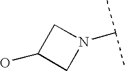 | IV.3 | 406 [M + H]⁺ | 2.29 (A) |
| 8.12 | 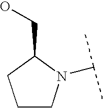 | IV.3 | 414 [M + H]⁺ | 2.31 (A) |
| 8.13 | 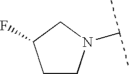 | IV.3 | 422 [M + H]⁺ | 2.33 (A) |
| 8.14 | 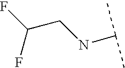 | IV.3 | 414 [M + H]⁺ | 2.34 (A) |
| 8.15 | 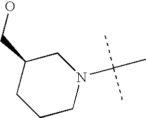 | IV.3 | 448 [M + H]⁺ | 2.30 (A) |

The following compounds of general formula IV can be prepared analogously to Example 7.1:

(IV)

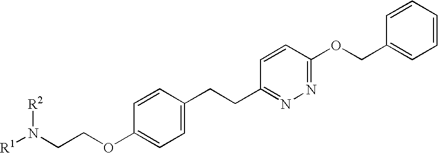

| Example | R¹R²N— |
|---|---|
| 8.16 | 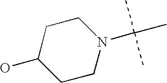 |
| 8.17 | 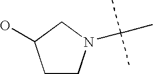 |
| 8.18 | 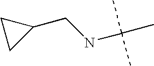 |
| 8.19 | 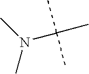 |
| 8.20 | 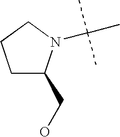 |
| 8.21 | 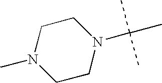 |
| 8.22 | 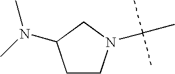 |
| 8.23 | 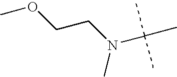 |
| 8.24 | 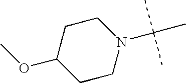 |
| 8.25 | 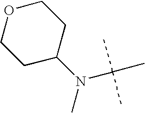 |
| 8.26 | 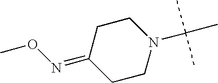 |
| 8.27 | 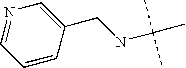 |
| 8.28 | 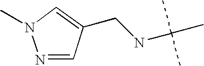 |
| 8.29 | 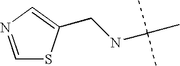 |
| 8.30 | 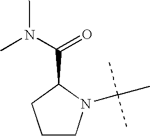 |
| 8.31 | 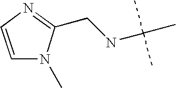 |
| 8.32 | 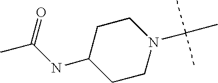 |
| 8.33 | 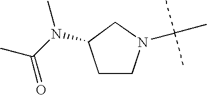 |
| 8.34 | 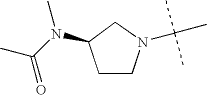 |
| 8.35 | 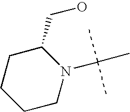 |
| 8.36 | 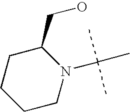 |
| 8.37 | 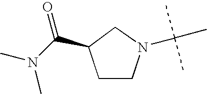 |
| 8.38 | 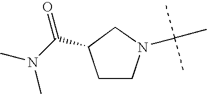 |
| 8.39 | 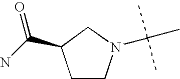 |
| 8.40 | 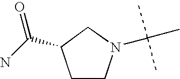 |
| 8.41 | 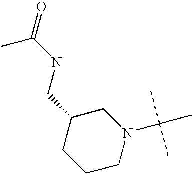 |
| 8.42 | 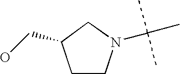 |

### Example 9.1

### 1-(4-{2-[6-(4-Fluoro-benzyloxy)-pyridazin-3-yl]-ethyl}-benzyl)-piperidin-4-ol

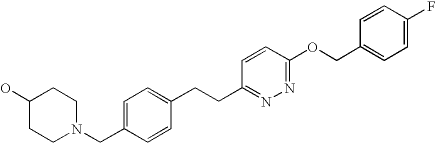

Prepared analogously to example 1.1 from 4-{2-[6-(4-fluoro-benzyloxy)-pyridazin-3-yl]-ethyl}-benzaldehyde (example 1.4) and 4-hydroxy-piperidine.

Yield: 230 mg (38% of theory),

retention time (HPLC): 2.58 min (method A)

C₂₅H₂₈FN₃O₂

EII mass spectrum: m/z=422 [M+H]⁺

### Example 10

The following compounds of general formula V are prepared analogously to Example 9.1, the educts used being shown in the column headed “Educts”:

(V)

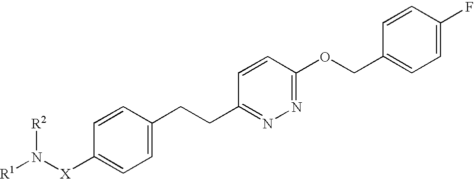

| Example | R¹R²N—X— | Educts | mass spectrum | Retention time (HPLC) |
|---|---|---|---|---|
| 10.1 | 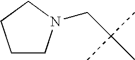 | I.4 | 392 [M + H]⁺ | 2.55 (A) |
| 10.2 | 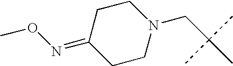 | I.4 and II.3 | 449 [M + H]⁺ | 2.55 (A) |
| 10.3 | 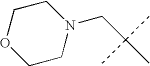 | I.4 | 408 [M + H]⁺ | 2.33 (A) |
| 10.4 | 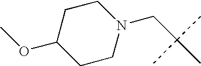 | I.4 | 436 [M + H]⁺ | 2.58 (A) |
| 10.5 | 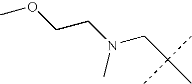 | I.4 | 410 [M + H]⁺ | 2.58 (A) |
| 10.6 | 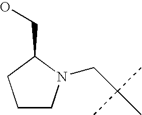 | I.4 | 422 [M + H]⁺ | 2.53 (A) |
| 10.7 | 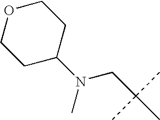 | I.4 | 436 [M + H]⁺ | 2.61 (A) |
| 10.8 | 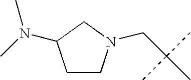 | I.4 | 449 [M + H]⁺ | 2.24 (A) |
| 10.9 | 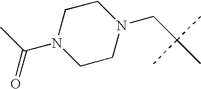 | I.4 | 449 [M + H]⁺ | 2.61 (A) |
| 10.10 | 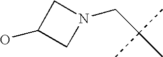 | I.4 | 394 [M + H]⁺ | 2.42 (A) |
| 10.11 | 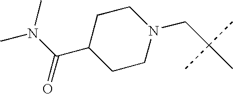 | I.4 | 477 [M + H]⁺ | 2.45 (A) |
| 10.12 | 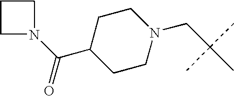 | I.4 and II.1 | 489 [M + H]⁺ | 2.30 (A) |
| 10.13 | 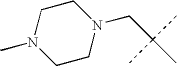 | I.4 | 421 [M + H]⁺ | 2.30 (A) |
| 10.14 | 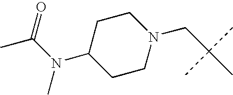 | I.4 and II.2 | 477 [M + H]⁺ | 2.28 (A) |
| 10.15 | 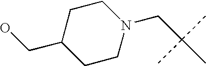 | I.4 | 436 [M + H]⁺ | 2.30 (A) |
| 10.16 | 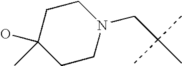 | I.4 | 436 [M + H]⁺ | 2.35 (A) |
| 10.17 | 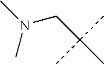 | I.4 | 366 [M + H]⁺ | 2.28 (A) |
| 10.18 | 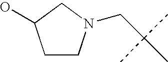 | I.4 | 408 [M + H]⁺ | 2.60 (A) |

The following compounds of general formula V can be prepared analogously to Example 9.1.

| | |
|---|---|
| | (V) |
| 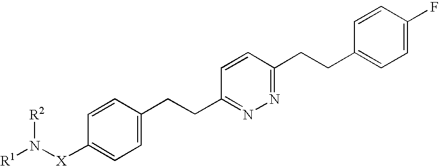 | |

| Example | R¹R²N—X— |
|---|---|
| 10.19 | 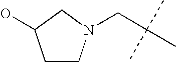 |
| 10.20 | 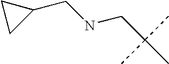 |
| 10.21 | 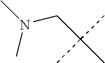 |
| 10.22 | 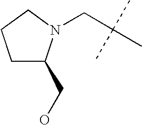 |
| 10.23 | 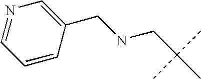 |
| 10.24 | 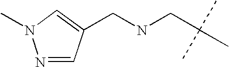 |
| 10.25 | 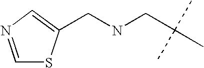 |
| 10.26 | 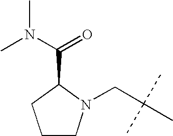 |
| 10.27 | 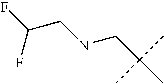 |
| 10.28 | 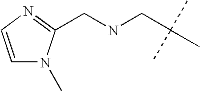 |
| 10.29 | 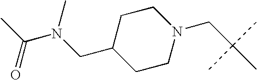 |
| 10.30 | 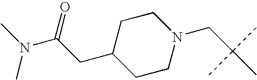 |
| 10.31 | 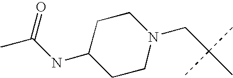 |
| 10.32 | 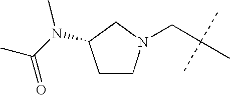 |
| 10.33 | 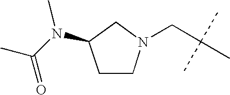 |
| 10.34 | 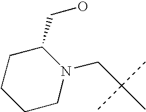 |
| 10.35 | 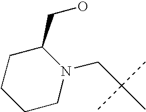 |
| 10.36 | 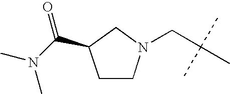 |
| 10.37 | 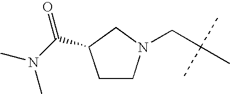 |
| 10.38 | 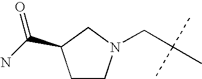 |
| 10.39 | 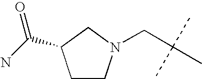 |
| 10.40 | 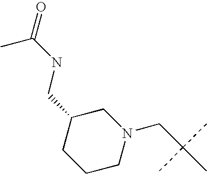 |
| 10.41 | 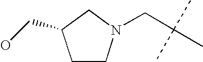 |
| 10.42 | 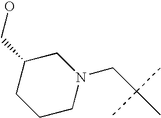 |
| 10.43 | 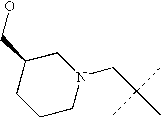 |

### Example 11.1

### 1-(4-{2-[6-(3-Fluoro-benzyloxy)-pyridazin-3-yl]-ethyl}-benzyl)-piperidin-4-ol trifluoroacetate

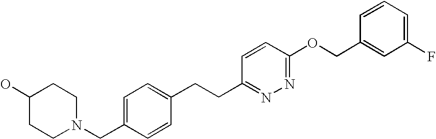

Prepared analogously to example 1.1 from 4-{2-[6-(3f-benzyloxy)-pyridazin-3-yl]-ethyl}-benzaldehyde (example 1.5) and 4-hydroxy-piperidine.

Yield: 80 mg (42% of theory),

retention time (HPLC): 2.56 min (method A)

C₂₅H₂₈FN₃O₂.C₂HF₃O₂

EII mass spectrum: m/z=422 [M+H]⁺

### Example 11.2

### 3-(3-Fluoro-benzyloxy)-6-[2-(4-pyrrolidin-1-ylmethyl-phenyl)-ethyl]-pyridazine trifluoroacetate

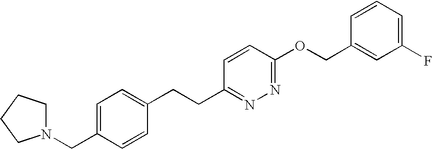

Prepared analogously to example 1.1 from 4-{2-[6-(3-fluoro-benzyloxy)-pyridazin-3-yl]-ethyl}-benzaldehyde (example 1.5) and pyrrolidine.

Yield: 125 mg (69% of theory),

retention time (HPLC): 2.44 min (method A)

C₂₄H₂₆FN₃O.C₂HF₃O₂

EII mass spectrum: m/z=392 [M+H]⁺

### Example 12.1

### 1-(4-{2-[6-(2-Fluoro-benzyloxy)-pyridazin-3-yl]-ethyl}-benzyl)-piperidin-4-ol

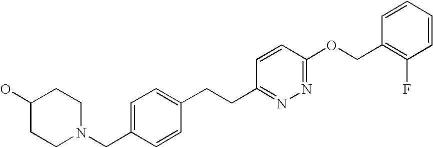

Prepared analogously to example 1.1 from 4-{2-[6-(3-fluoro-benzyloxy)-pyridazin-3-yl]-ethyl}-benzaldehyde (example 1.6) and 4-hydroxy-piperidine.

Yield: 40 mg (32% of theory),

retention time (HPLC): 2.33 min (method A)

C₂₆H₂₈FN₃O₂

EII mass spectrum: m/z=422 [M+H]⁺

### Example 12.2

### 3-(2-Fluoro-benzyloxy)-6-[2-(4-pyrrolidin-1-ylmethyl-phenyl)-ethyl]-pyridazine trifluoroacetate

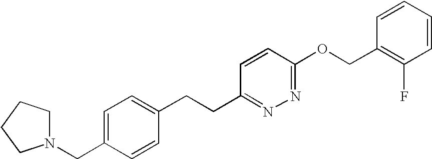

Prepared analogously to example 1.1 from 4-{2-[6-(3-Fluoro-benzyloxy)-pyridazin-3-yl]-ethyl}-benzaldehyde (1.5) and pyrrolidine.

Yield: 45 mg (39% of theory),

retention time (HPLC): 2.38 min (method A)

C₂₄H₂₆FN₃O

EII mass spectrum: m/z=392 [M+H]⁺

### Example 13.1

### 1-(4-{2-[6-(Thiazol-2-ylmethoxy)-pyridazin-3-yl]-ethyl}-benzyl)-piperidin-4-o2 trifluoroacetate

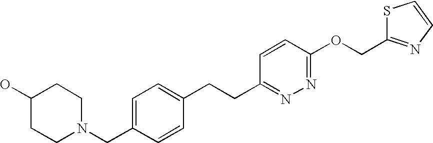

Prepared analogously to example 1.1 from 4-{2-[6-(thiazol-2-ylmethoxy)-pyridazin-3-yl]-ethyl}-benzaldehyde (example 1.3) and 4-hydroxy-piperidine.

Yield: 20 mg (15% of theory),

C₂₂H₂₆N₄O₂S.C₂HF₃O₂

EII mass spectrum: m/z=411 [M+H]⁺

### Example 13.2

### 3-[2-(4-Pyrrolidin-1-ylmethyl-phenyl)-ethyl]-6-(thiazol-2-ylmethoxy)-pyridazine trifluoroacetate

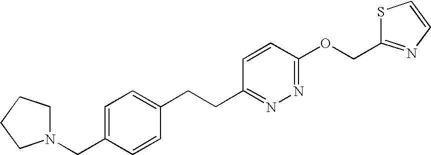

Prepared analogously to example 1.1 from 4-{2-[6-(thiazol-2-ylmethoxy)-pyridazin-3-yl]-ethyl}-benzaldehyde (1.3) and pyrrolidine.

Yield: 40 mg (33% of theory),

retention time (HPLC): 2.15 min (method A)

C₂₁H₂₄N₄OS.C₂HF₃O₂

EII mass spectrum: m/z=381 [M+H]⁺

### Example 14.1

### 3-(Pyridin-2-ylmethoxy)-6-[2-(4-pyrrolidin-1-ylmethyl-phenyl)-ethyl]-pyridazine trifluoroacetate

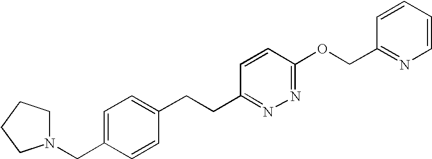

Prepared analogously to example 1.1 from 4-{2-[6-(pyridin-2-ylmethoxy)-pyridazin-3-yl]-ethyl}-benzaldehyde (example 1.2) and pyrrolidine.

Yield: 90 mg (73% of theory),

retention time (HPLC): 2.01 min (method A)

C₂₃H₂₆N₄O.C₂HF₃O₂

EII mass spectrum: m/z=372 [M+H]⁺

### Example 14.2

### 1-(4-{2-[6-(Pyridin-2-ylmethoxy)-pyridazin-3-yl]-ethyl}-benzyl)-piperidin-4-ol trifluoroacetate

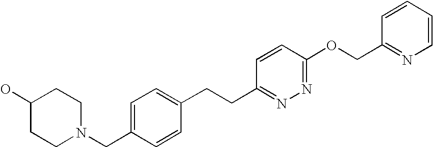

Prepared analogously to example 1.1 from 4-{2-[6-(pyridin-2-ylmethoxy)-pyridazin-3-yl]-ethyl}-benzaldehyde (example 1.2) and 4-hydroxy-piperidine.

Yield: 30 mg (23% of theory),

retention time (HPLC): 1.91 min (method A)

C₂₄H₂₈N₄O₂.C₂HF₃O₂

EII mass spectrum: m/z=405 [M+H]⁺

### Example 15.1

### 2-[6-(4-Fluoro-benzyloxy)-pyridazin-3-yl]-1-[4-(4-methoxy-piperidin-1-ylmethyl)-phenyl]-ethanone

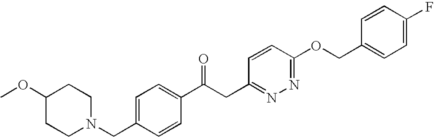

A reaction mixture of 110 mg (0.25 mmol) 3-(4-fluoro-benzyloxy)-6-[4-(4-methoxy-piperidin-1-ylmethyl)-phenylethynyl]-pyridazine, 1 ml trifluoro acetic acid, 1 ml water and 5 mg mercury(II) sulphate is stirred for 8 hours at 50° C. The reaction mixture is concentrated. Methylene chloride is added to the residue and the resulting mixture is extracted with water. The organic phase is collected by passing the reaction mixture through a column (Phase Separator/Separtis) and concentrated. Purification is achieved by HPLC (method 3). The purified product is dissolved in methylene chloride and passed through a column

(StratoSpheres SPE PL-HCO3 MP Resin) and concentrated.

Yield: 19 mg (13% of theory),

retention time (HPLC): 2.45 min (method A)

C₂₆H₂₈FN₃O₃

EII mass spectrum: m/z=450 [M+H]⁺

### Example 15.2

### 2-(6-Benzyloxy-pyridazin-3-yl)-1-[4-(4-hydroxy-4-methyl-piperidin-1-ylmethyl)-phenyl]-ethanone

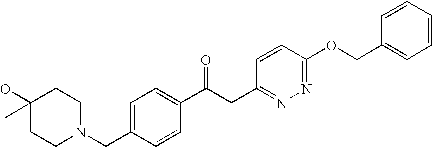

Prepared analogously to example 15.1 from 1-[4-(6-Benzyloxy-pyridazin-3-ylethynyl)-benzyl]-4-methyl-piperidin-4-ol.

Yield: 2 mg (1% of theory),

retention time (HPLC): 2.17 min (method A)

C₂₆H₂₉N₃O₃

EII mass spectrum: m/z=432 [M+H]⁺

The following compounds of general formula VI can be prepared analogously to Example 15.1

| | |
|---|---|
| | (VI) |
| 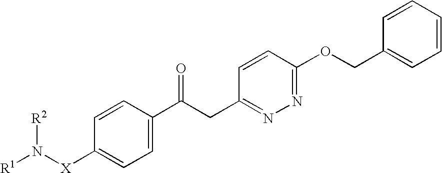 | |

| Example | R¹R²N—X— |
|---|---|
| 15.3 | 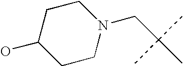 |
| 15.4 | 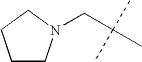 |
| 15.5 | 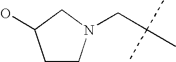 |
| 15.6 | 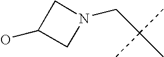 |
| 15.7 | 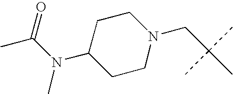 |
| 15.8 | 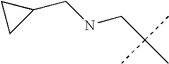 |
| 15.9 | 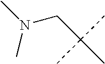 |
| 15.10 | 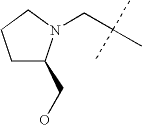 |
| 15.11 | 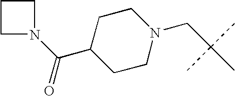 |
| 15.12 | 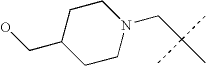 |
| 15.13 | 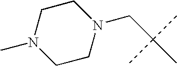 |
| 15.14 | 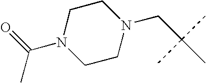 |
| 15.15 | 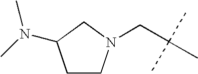 |
| 15.16 | 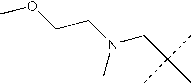 |
| 15.17 | 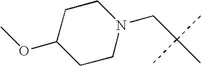 |
| 15.18 | 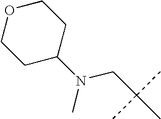 |
| 15.19 | 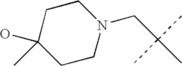 |
| 15.20 | 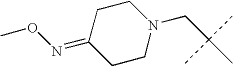 |
| 15.21 | 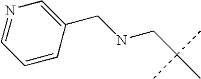 |
| 15.22 | 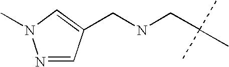 |
| 15.23 | 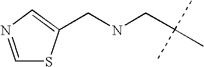 |
| 15.24 | 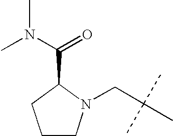 |
| 15.25 | 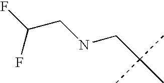 |
| 15.26 | 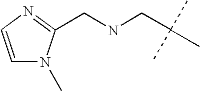 |
| 15.27 | 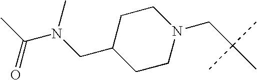 |
| 15.28 | 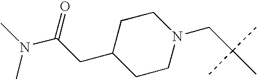 |
| 15.29 | 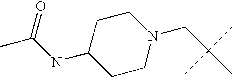 |
| 15.30 | 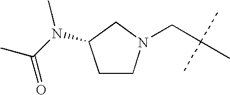 |
| 15.31 | 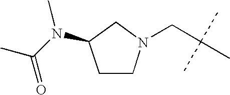 |
| 15.32 | 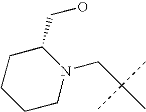 |
| 15.33 | 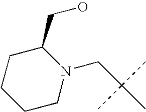 |
| 15.34 | 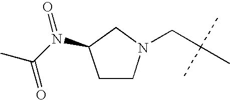 |
| 15.35 | 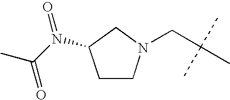 |
| 15.36 | 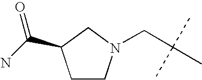 |
| 15.37 | 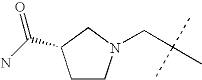 |
| 15.38 | 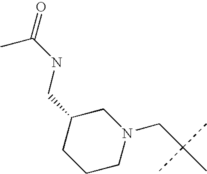 |
| 15.39 | 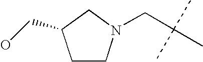 |
| 15.40 | 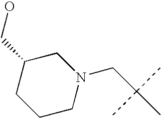 |
| 15.41 | 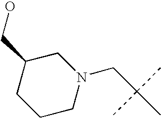 |

The following compounds can be prepared analogously to Example 15.1

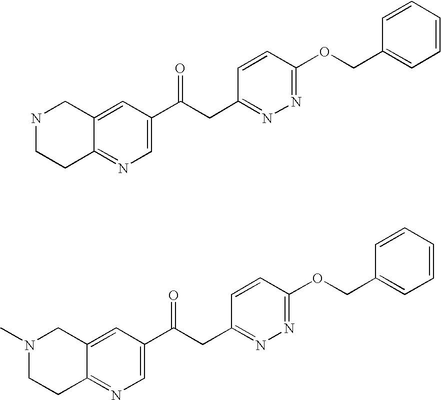

### Example 16.1

### Benzyl-{6-[2-(4-pyrrolidin-1-ylmethyl-phenyl)-ethyl]-pyridazin-3-yl}-amine

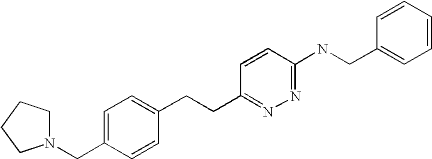

Prepared analogously to example 1.1 from 4-[2-(6-benzylamino-pyridazin-3-yl)-ethyl]-benzaldehyde (example 1.10) and pyrrolidine.

Yield: 30 mg (17% of theory),

retention time (HPLC): 1.74 min (method A)

C₂₄H₂₈N₄

EII mass spectrum: m/z=373 [M+H]⁺

### Example 16.2

### Benzyl-methyl-{6-[2-(4-pyrrolidin-1-ylmethyl-phenyl)-ethyl]-pyridazin-3-yl}-amine

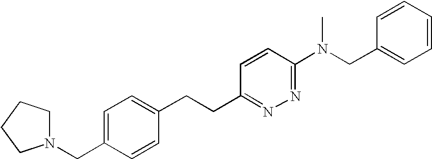

Prepared analogously to example 1.1 from 4-{2-[6-(benzyl-methyl-amino)-pyridazin-3-yl]-ethyl}-benzaldehyde (example 1.11) and pyrrolidine.

Yield: 102 mg (44% of theory),

retention time (HPLC): 1.86 min (method A)

C₂₅H₃₀N₄

EII mass spectrum: m/z=387 [M+H]⁺

### Example 17.1

### 3-Benzyloxy-6-[2-(4-pyrrolidin-1-ylmethyl-thiazol-2-yl)-ethyl]-pyridazine

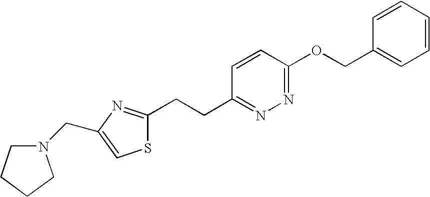

Prepared analogously to example 7.1 from methanesulfonic acid 2-[2-(6-benzyloxy-pyridazin-3-yl)-ethyl]-thiazol-4-ylmethyl ester (example 1.12) and pyrrolidine.

Yield: 8 mg (28% of theory),

retention time (HPLC): 2.3 min (method A)

C₂₁H₂₄N₄OS

EII mass spectrum: m/z=381 [M+H]⁺

### Example 18.1

### {5-[2-(6-Benzyloxy-pyridazin-3-yl)-ethyl]-pyridin-2-yl}-(2-pyrrolidin-1-yl-ethyl)-amine

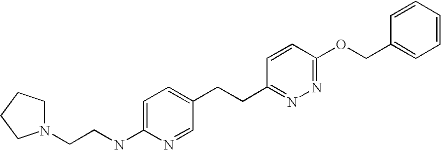

Prepared analogously to example 1.1.d from [5-(6-benzyloxy-pyridazin-3-ylethynyl)-pyridin-2-yl]-(2-pyrrolidin-1-yl-ethyl)-amine (example V.2.).

Yield: 50 mg (70% of theory),

retention time (HPLC): 2.13 min (method A)

C₂₄H₂₉N₅O

EII mass spectrum: m/z=404 [M+H]⁺

### Example 19.1

### 3-Benzyloxy-6-{2-[6-(2-pyrrolidin-1-yl-ethoxy)-pyridin-3-yl]-ethyl}-pyridazine trifluoroacetate

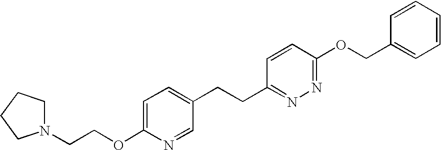

Prepared analogously to example 1.1.d from 3-benzyloxy-6-[6-(2-pyrrolidin-1-yl-ethoxy)-pyridin-3-ylethynyl]-pyridazine (example V.3.).

Yield: 25 mg (16% of theory),

retention time (HPLC): 2.35 min (method A)

C₂₄H₂₈N₄O₂.C₂HF₃O₂

EII mass spectrum: m/z=405 [M+H]⁺

The following compounds of general formula VII can be prepared analogously to Example 19.1.

| | |
|---|---|
| | (VII) |
| 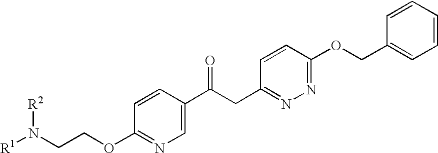 | |

| Example | R¹R²N— |
|---|---|
| 19.2 | 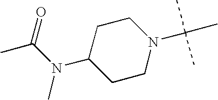 |
| 19.3 | 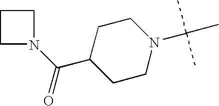 |
| 19.4 | 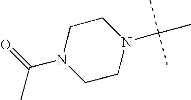 |
| 19.5 | 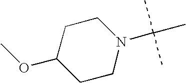 |
| 19.6 | 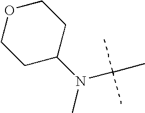 |
| 19.7 | 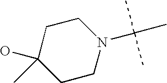 |
| 19.8 | 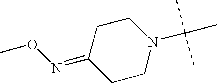 |
| 19.9 | 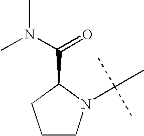 |
| 19.10 | 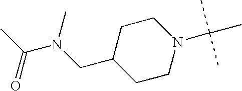 |
| 19.11 | 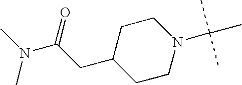 |
| 19.12 | 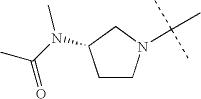 |
| 19.13 | 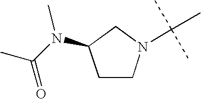 |
| 19.14 | 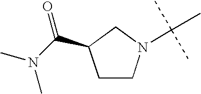 |
| 19.15 | 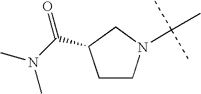 |

### Example 20.1

### 3-[2-(4-Pyrrolidin-1-ylmethyl-phenyl)-ethyl]-6-(thiophen-2-ylmethoxy)-pyridazine

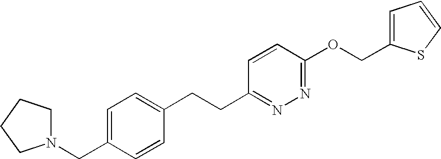

Prepared analogously to example 1.1. from 4-{2-[6-(thiophen-2-ylmethoxy)-pyridazin-3-yl]-ethyl}-benzaldehyde (1.7) and pyrrolidine.

Yield: 40 mg (34% of theory),

retention time (HPLC): 2.27 min (method A)

C₂₂H₂₅N₃OS

EII mass spectrum: m/z=380 [M+H]⁺

The following compounds of general formula VI are prepared analogously to Example 20.1, the educts used being shown in the column headed “Educts”:

| | |
|---|---|
| | (VI) |
| 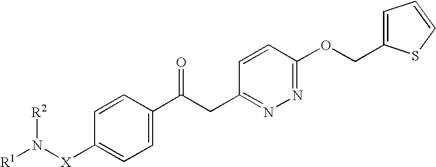 | |

| Exam- | | | mass | Retention time |
|---|---|---|---|---|
| ple | R¹R²N—X— | Educts | spectrum | (HPLC) |
| 20.2 | 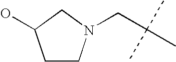 | I.7 | 396 [M + H]⁺ | 2.23 (A) |
| 20.3 | 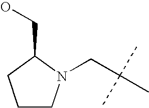 | I.7 | 410 [M + H]⁺ | 2.23 (A) |
| 20.4 | 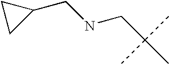 | I.7 | 380 [M + H]⁺ | 2.33 (A) |
| 20.5 | 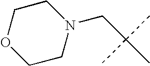 | I.7 | 396 [M + H]⁺ | 2.23 (A) |
| 20.6 | 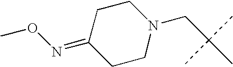 | I.7 and II.3 | 437 [M + H]⁺ | 2.36 (A) |

### Example 21.1

### 3-(3-Methyl-butoxy)-6-[2-(4-pyrrolidin-1-ylmethyl-phenyl)-ethyl]-pyridazine

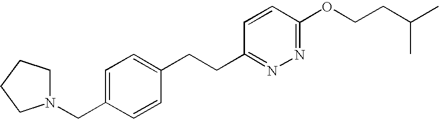

Prepared analogously to example 1.1. from 4-{2-[6-(3-methyl-butoxy)-pyridazin-3-yl]-ethyl}-benzaldehyde (example 1.8) and pyrrolidine.

Yield: 118 mg (30% of theory),

retention time (HPLC): 2.42 min (method A)

C₂₂H₃₁N₃O

EII mass spectrum: m/z=354 [M+H]⁺

### Example 21.2

### 1-(4-{2-[6-(3-Methyl-butoxy)-pyridazin-3-yl]-ethyl}-benzyl)-piperidin-4-ol

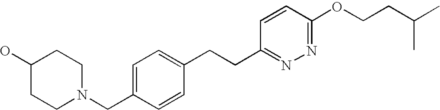

Prepared analogously to example 1.1. from 4-{2-[6-(3-methyl-butoxy)-pyridazin-3-yl]-ethyl}-benzaldehyde (example 1.8) and 4-hydroxy-piperidine.

Yield: 149 mg (35% of theory),

retention time (HPLC): 2.28 min (method A)

C₂₂H₃₃N₃O₂

EII mass spectrum: m/z=384 [M+H]⁺

### Example 22.1

### 3-[2-(4-Pyrrolidin-1-ylmethyl-phenyl)-ethyl]-6-(tetrahydro-pyran-2-ylmethoxy)-pyridazine

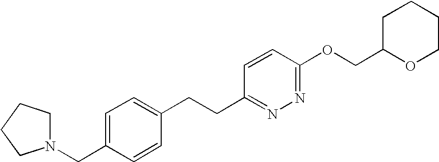

Prepared analogously to example 1.1. from 4-{2-[6-(tetrahydro-pyran-2-ylmethoxy)-pyridazin-3-yl]-ethyl}-benzaldehyde (example 1.9) and pyrrolidine.

Yield: 65 mg (46% of theory),

retention time (HPLC): 2.03 min (method A)

C₂₃H₃₁N₃O₂

EII mass spectrum: m/z=382 [M+H]⁺

### Example 22.2

### 1-(4-{2-[6-(Tetrahydro-pyran-2-ylmethoxy)-pyridazin-3-yl]-ethyl}-benzyl)-piperidin-4-ol

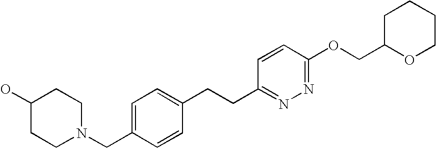

Prepared analogously to example 1.1. from 4-{2-[6-(tetrahydro-pyran-2-ylmethoxy)-pyridazin-3-yl]-ethyl}-benzaldehyde (1.9) and 4-hydroxy-piperidine.

Yield: 55 mg (36% of theory),

retention time (HPLC): 2.03 min (method A)

C₂₄H₃₃N₃O₃

EII mass spectrum: m/z=412 [M+H]⁺

### Example 23.1

### 3-(1-Phenyl-ethoxy)-6-[2-(4-pyrrolidin-1-ylmethyl-phenyl)-ethyl]-pyridazine

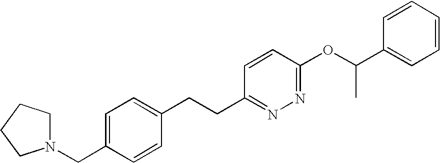

0.142 ml (1.7 mmol) pyrrolidine are added to a solution of 0.2 g (0.56 mmol) 3-[2-(4-chloromethyl-phenyl)-ethyl]-6-(1-phenyl-ethoxy)-pyridazine (example III.3) in 5 ml dry THF. The reaction mixture is stirred at 50° C. for 18 hours. Then the reaction mixture is poured in water and extracted with EtOAc. The organic phase is extracted with water three times, dried over sodium sulphate/activated carbon and concentrated. Purification is achieved by silica gel column chromatography with methylene chloride/methanol as eluent.

Yield: 15 mg (7% of theory),

retention time (HPLC): 2.75 min (method A)

C₂₅H₂₉N₃O

EII mass spectrum: m/z=388 [M+H]⁺

### Example 23.2

### 1-(4-{2-[6-(1-Phenyl-ethoxy)-pyridazin-3-yl]-ethyl}-benzyl)-piperidin-4-ol

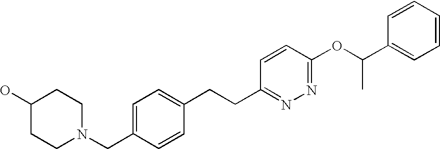

Prepared analogously to 23.2 from 3-[2-(4-chloromethyl-phenyl)-ethyl]-6-(1-phenyl-ethoxy)-pyridazine (example III.3) and 4-hydroxy-piperidine.

Yield: 30 mg (12% of theory),

retention time (HPLC): 2.55 min (method A)

C₂₆H₃₁N₃O₂

EII mass spectrum: m/z=418 [M+H]⁺

### Example 24.1

### 7-[2-(6-Benzyloxy-pyridazin-3-yl)-ethyl]-2,3,4,5-tetrahydro-1H-benzo[d]azepine

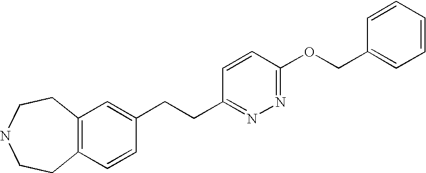

To a mixture of 400 mg (0.88 mmol) 1-{7-[2-(6-benzyloxy-pyridazin-3-yl)-ethyl]-1,2,4,5-tetrahydro-benzo[d]azepin-3-yl}-2,2,2-trifluoro-ethanone and 10 ml methanol are added 2 ml 1N sodium hydroxide solution at room temperature. The reaction mixture is stirred for 18 hours and concentrated until half of the solvent is evaporated. Water is added to the residue. The mixture is filtered. The remaining solid is dried and washed with diisopropylether.

Yield: 260 mg (82% of theory),

retention time (HPLC): 2.46 min (method A)

C₂₃H₂₅N₃O

EII mass spectrum: m/z=360 [M+H]⁺

### Example 24.2

### 7-[2-(6-Benzyloxy-pyridazin-3-yl)-ethyl]-3-methyl-2,3,4,5-tetrahydro-1H-benzo[d]azepine

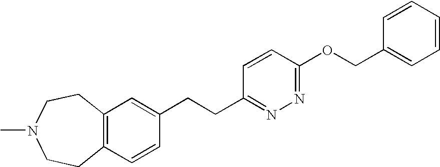

17.46 μl (0.27 mmol) methyl iodide are added at room temperature to a mixture of 100 mg (0.27 mmol) 7-[2-(6-benzyloxy-pyridazin-3-yl)-ethyl]-2,3,4,5-tetrahydro-1H-benzo[d]azepine and 76 mg (0.55 mmol) potassium carbonate in 5 ml of acetone. The reaction mixture is stirred for two hours and filtered. The filtrate is concentrated. The residue is extracted with methylene chloride and water. The organic phase is dried and concentrated. Purification is achieved by silica gel column chromatography with methylene chloride/methanol/ammonia in water as eluent.

Yield: 21 mg (20% of theory),

retention time (HPLC): 2.61 min (method A)

C₂₄H₂₇N₃O

EII mass spectrum: m/z=374 [M+H]⁺

### 4-{2-[6-(2-Thiophen-3-yl-ethyl)-pyridazin-3-yl]-ethyl}-benzaldehyde

### Example 25.1

### Dimethyl-(4-{2-[6-(2-thiophen-3-yl-ethyl)-pyridazin-3-yl]-ethyl}-benzyl)-amine trifluoroacetate

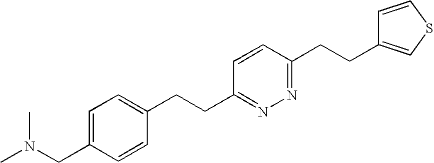

Prepared analogously to example 1.1 from 4-{2-[6-(2-thiophen-3-yl-ethyl)-pyridazin-3-yl]-ethyl}-benzaldehyde (example 1.13) and dimethylamine.

Yield: 101 mg (70% of theory),

retention time (HPLC): 2.12 min (method A)

C₂₁H₂₅N₃S.C₂HF₃O₂

EII mass spectrum: m/z=352 [M+H]⁺

### Example 25.2

### 1-(4-{2-[6-(2-Thiophen-3-yl-ethyl)-pyridazin-3-yl]-ethyl}-benzyl)-piperidin-4-ol trifluoroacetate

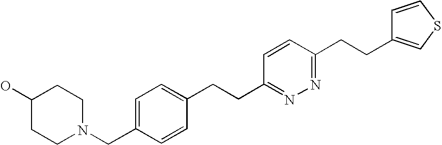

Prepared analogously to example 1.1 from 4-{2-[6-(2-thiophen-3-yl-ethyl)-pyridazin-3-yl]-ethyl}-benzaldehyde (example 1.13) and 4-hydroxy-piperidine.

Yield: 63 mg (39% of theory),

retention time (HPLC): 2.36 min (method A)

C₂₄H₂₉N₃OS.C₂HF₃O₂

EII mass spectrum: m/z=408 [M+H]⁺

### Example 26.1

### 3-Benzyloxy-6-[2-(6-pyrrolidin-1-ylmethyl-pyridin-3-yl)-ethyl]-pyridazine

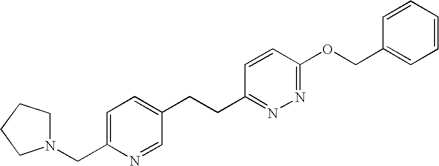

A mixture of 0.1 g (0.29 mmol) 3-Benzyloxy-6-[2-(6-chloromethyl-pyridin-3-yl)-ethyl]-pyridazine and 83 mg (1.1 mmol) pyrrolidine in 3 ml DMF is stirred for 2 hours at room temperature. Purification is achieved by HPLC (method 2). Product containing fractions are combined, adjusted to basic pH by addition of sodium hydroxide solution (2N) and extracted with methylene chloride.

Yield: 90 mg (82% of theory),

retention time (HPLC): 2.27 min (method A)

C₂₃H₂₆N₄O

EII mass spectrum: m/z=374 [M+H]⁺

The following compounds of general formula VII are prepared analogously to Example 26.1;

the educts used being shown in the column headed “Educts”:

| | |
|---|---|
| | (VII) |
| 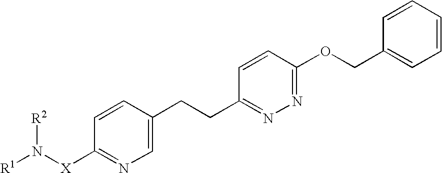 | |

| Exam- | | | mass | Retention time |
|---|---|---|---|---|
| ple | R¹R²N—X— | Educts | spectrum | (HPLC) |
| 26.2 | 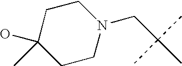 | III.4 | 419 [M + H]⁺ | 2.13 (A) |
| 26.3 | 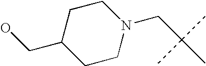 | III.4 | 419 [M + H]⁺ | 2.03 (A) |
| 26.4 | 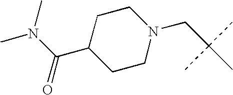 | III.4 | 460 [M + H]⁺ | 2.10 (A) |
| 26.6 | 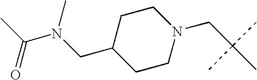 | III.4 | 474 [M + H]⁺ | 2.04 (A) |

### Example 27.1

### 7-[2-(6-Benzyloxy-pyridazin-3-yl)-ethyl]-1,2,3,4-tetrahydro-isoquinoline

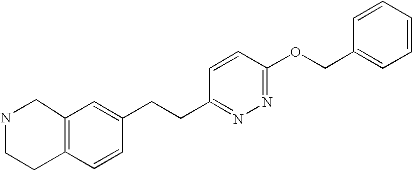

A mixture of 0.55 g (1.23 mmol) 7-[2-(6-Benzyloxy-pyridazin-3-yl)-ethyl]-3,4-dihydro-1H-isoquinoline-2-carboxylic acid tert-butyl ester and 1.5 ml Trifluoroacetic acid in 50 ml of methylene chloride is stirred for one hour at room temperature. The mixture is adjusted to a basic pH with 2N aqueous sodium hydroxide solution. The organic phase is extracted with water and dried over sodium sulphate.

Yield: 380 mg (89% of theory),

retention time (HPLC): 2.17 min (method A)

C₂₂H₂₃N₃O

EII mass spectrum: m/z=346 [M+H]⁺

### Example 27.2

### 2-Methyl-7-{2-[6-((3Z,5Z)-2-methylene-hepta-3,5-dienyloxy)-pyridazin-3-yl]-ethyl}-1,2,3,4-tetrahydro-isoquinoline

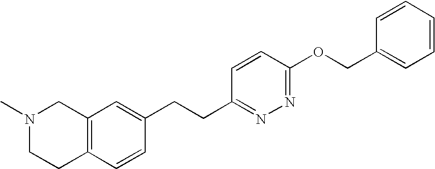

To a mixture of 0.15 g (0.434 mmol) 7-[2-(6-Benzyloxy-pyridazin-3-yl)-ethyl]-1,2,3,4-tetrahydro-isoquinoline and 0.07 ml formaldehyde solution (37% in water) in 6 ml THF are added 1.05 ml sodium citrate buffer solution (pH 5) and 0.109 mg (0.516 mmol) sodium triacetoxyborohydride. The mixture is stirred for two hours at room temperature. Then water and methylene chloride are added. The organic phase is separated. The water phase is extracted with methylene chloride. The combined organic phases are extracted three times with water and are dried over sodium sulphate. Purification is achieved by HPLC (method 2). Product containing fractions are combined, adjusted to basic pH by addition of sodium hydroxide solution (2N) and extracted with methylene chloride.

Yield: 70 mg (45% of theory),

retention time (HPLC): 2.13 min (method A)

C₂₃H₂₅N₃O

EII mass spectrum: m/z=360 [M+H]⁺

### Example 27.3

### 6-[2-(6-Benzyloxy-pyridazin-3-yl)-ethyl]-1,2,3,4-tetrahydro-isoquinoline

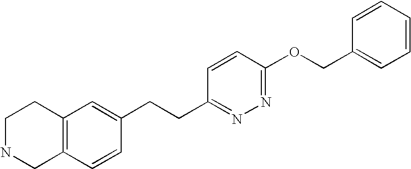

Prepared analogously to example III.1.d from 6-(6-Benzyloxy-pyridazin-3-ylethynyl)-1,2,3,4-tetrahydro-isoquinoline trifluoroacetate.

Yield: 198 mg (87% of theory),

retention time (HPLC): 2.14 min (method A)

C₂₃H₂₅N₃O

EII mass spectrum: m/z=346 [M+H]⁺

### Example 27.4

### 2-(6-Benzyloxy-pyridazin-3-yl)-1-(1,2,3,4-tetrahydro-isoquinolin-6-yl)-ethanone

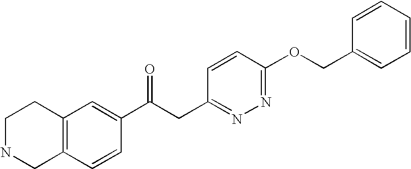

Prepared analogously to example 15.1 from 6-(6-Benzyloxy-pyridazin-3-ylethynyl)-1,2,3,4-tetrahydro-isoquinoline.

Yield: 85 mg (36% of theory),

retention time (HPLC): 2.18 min (method A)

C₂₃H₂₁N₃O₂

EII mass spectrum: m/z=360 [M+H]⁺

### Example 27.5

### 6-[2-(6-Benzyloxy-pyridazin-3-yl)-ethyl]-2-methyl-1,2,3,4-tetrahydro-isoquinoline

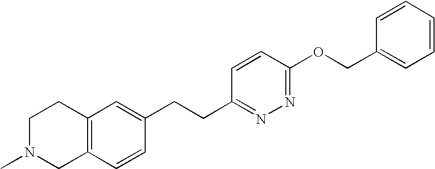

Prepared analogously to example 27.2 from 6-[2-(6-Benzyloxy-pyridazin-3-yl)-ethyl]-1,2,3,4-tetrahydro-isoquinoline.

Yield: 65 mg (52% of theory),

retention time (HPLC): 2.27 min (method A)

C₂₃H₂₅N₃O

EII mass spectrum: m/z=360 [M+H]⁺

### Example 28.1

### 3-Phenethyl-6-[2-(5-pyrrolidin-1-ylmethyl-pyridin-2-yl)-ethyl]-pyridazine

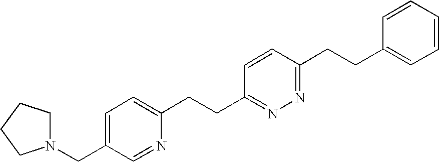

Prepared analogously to example 3.2 from 3-[2-(5-Chloromethyl-pyridin-2-yl)-ethyl]-6-phenethyl-pyridazine and pyrrolidine.

Yield: 40 mg (23% of theory),

retention time (HPLC): 1.94 min (method A)

C₂₄H₂₈N₄

EII mass spectrum: m/z=373 [M+H]⁺

The following compounds of general formula VIII are prepared analogously to Example 3.2; the educts used being shown in the column headed “Educts”:

| | |
|---|---|
| | (VIII) |
| 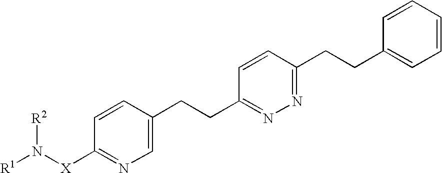 | |

| | | | | Retention |
|---|---|---|---|---|
| Exam- | | | mass | time |
| le | R¹R²N—X— | Educts | spectrum | (HPLC) |
| 28.2 | 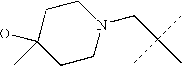 | III.5 | 417 [M + H]⁺ | 1.90 (A) |
| 28.3 | 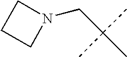 | III.5 | 359 [M + H]⁺ | 1.90 (A) |
| 28.4 | 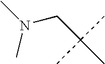 | III.5 | 347 [M + H]⁺ | 1.93 (A) |

### Example 29.1

### 3-Benzyloxy-6-{2-[5-(2-pyrrolidin-1-yl-ethoxy)-pyridin-2-yl]-ethyl}-pyridazine

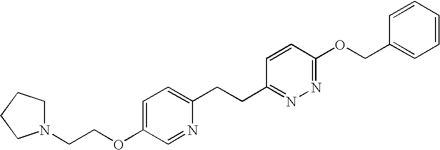

Prepared analogously to example 7.1 from Methanesulfonic acid 2-{6-[2-(6-benzyloxy-pyridazin-3-yl)-ethyl]-pyridin-3-yloxy}-ethyl ester and pyrrolidine.

Yield: 188 mg (80% of theory),

retention time (HPLC): 1.94 min (method A)

C₂₄H₂₈N₄O₂

EII mass spectrum: m/z=405 [M+H]⁺

The following compounds of general formula IX are prepared analogously to Example 7.1; the educts used being shown in the column headed “Educts”:

| | |
|---|---|
| | (IX) |
| 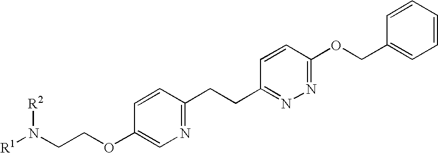 | |

| | | | | Retention |
|---|---|---|---|---|
| | | E- | mass | time |
| Example | R¹R²N— | ducts | spectrum | (HPLC) |
| 29.2 | 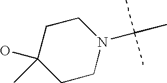 | IV.4 | 449 [M + H]⁺ | 1.90 (A) |
| 29.3 | 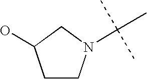 | IV.4 | 421 [M + H]⁺ | 1.96 (A) |
| 29.4 | 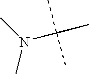 | IV.4 | 379 [M + H]⁺ | 1.86 (A) |
| 29.5 | 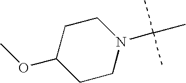 | IV.4 | 449 [M + H]⁺ | 1.88 (A) |
| 29.6 | 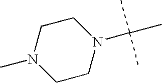 | IV.4 | 434 [M + H]⁺ | 1.81 (A) |
| 29.7 | 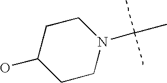 | IV.4 | 435 [M + H]⁺ | 1.85 (A) |
| 29.8 | 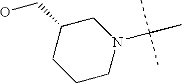 | IV.4 | 449 [M + H]⁺ | 1.88 (A) |
| 29.9 | 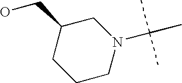 | IV.4 | 449 [M + H]⁺ | 1.90 (A) |
| 29.10 | 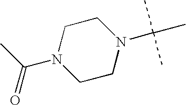 | IV.4 | 462 [M + H]⁺ | 1.89 (A) |
| 29.11 | 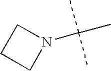 | IV.4 | 391 [M + H]⁺ | 2.01 (A) |

The following compounds of general formula IX can be prepared analogously to Example 7.1

| | |
|---|---|
| | (IX) |
| 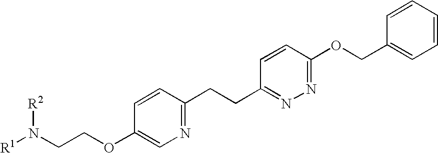 | |

| Example | R¹R²N— |
|---|---|
| 29.12 | 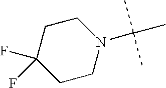 |
| 29.13 | 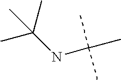 |
| 29.14 | 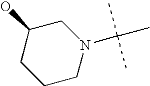 |
| 29.15 | 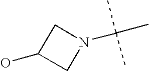 |
| 29.16 | 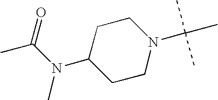 |
| 29.17 | 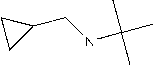 |
| 29.18 | 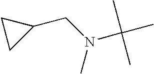 |
| 29.19 | 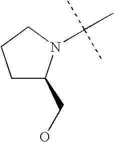 |
| 29.20 | 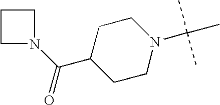 |
| 29.21 | 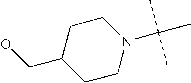 |
| 29.22 | 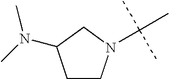 |
| 29.23 | 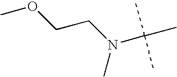 |
| 29.24 | 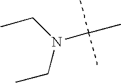 |
| 29.25 | 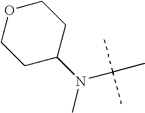 |
| 29.26 | 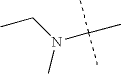 |
| 29.27 | 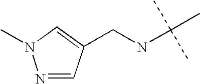 |
| 29.28 | 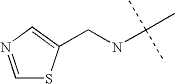 |
| 29.29 | 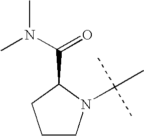 |
| 29.30 | 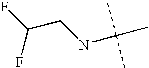 |
| 29.31 | 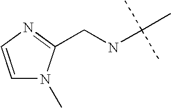 |
| 29.32 | 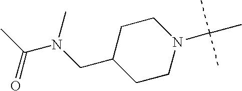 |
| 29.33 | 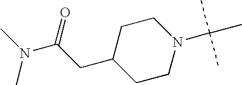 |
| 29.34 | 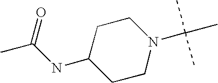 |
| 29.35 | 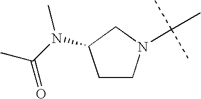 |
| 29.36 | 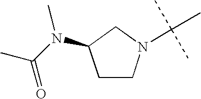 |
| 29.37 | 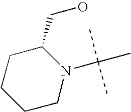 |
| 29.38 | 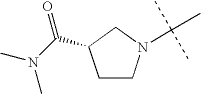 |
| 29.39 | 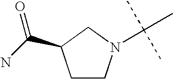 |
| 29.40 | 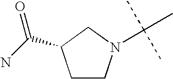 |
| 29.41 | 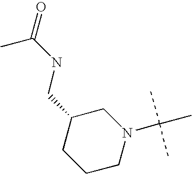 |
| 29.42 | 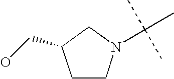 |
| 29.43 | 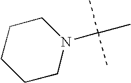 |

### Example 30.1

### 1-(2-{6-[2-(6-Benzyloxy-pyridazin-3-yl)-ethyl]-pyridin-3-yl}-ethyl)-azetidin-3-ol

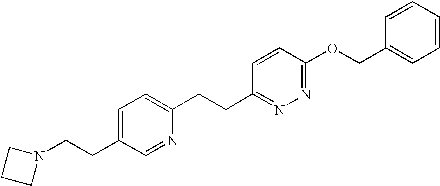

Prepared analogously to example 7.1 from Methanesulfonic acid 2-{6-[2-(6-benzyloxy-pyridazin-3-yl)-ethyl]-pyridin-3-yl}-ethyl ester and Azetidin-3-ol in acetonitrile.

Yield: 80 mg (19% of theory),

retention time (HPLC): 1.83 min (method A)

C₂₃H₂₆N₄O₂

EII mass spectrum: m/z=391 [M+H]⁺

### Example 30.2

### 1-(2-{6-[2-(6-Benzyloxy-pyridazin-3-yl)-ethyl]-pyridin-3-yl}-ethyl)-4-methyl-piperidin-4-ol

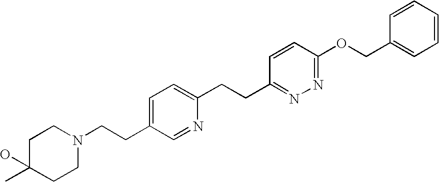

A mixture of 0.3 g (0.848 mmol) 3-Benzyloxy-6-{2-[5-(2-chloro-ethyl)-pyridin-2-yl]-ethyl}-pyridazine and 0.196 g (1.7 mmol) 4-Methyl-piperidin-4-ol in 3 ml acetonitrile is stirred at 70° C. for 18 hours. The mixture is purified by HPLC (method 3).

Yield: 80 mg (19% of theory),

retention time (HPLC): 1.85 min (method A)

C₂₆H₃₂N₄O₂

EII mass spectrum: m/z=433 [M+H]⁺

### Example 31

The following compounds of general formula X are prepared analogously to Example 30.1 or 30.2; the educts used being shown in the column headed “Educts”:

| | |
|---|---|
| | (X) |
| 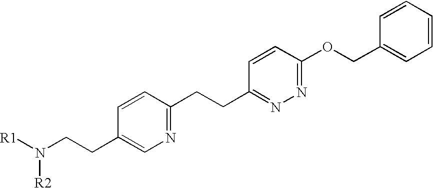 | |

| | | | | Retention |
|---|---|---|---|---|
| | | E- | mass | time |
| Example | R¹R²N— | ducts | spectrum | (HPLC) |
| 31.1 | 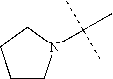 | III.7 | 389 [M + H]⁺ | 1.82 (A) |
| 31.2 | 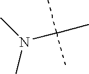 | III.7 | 363 [M + H]⁺ | 1.82 (A) |
| 31.3 | 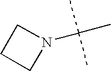 | III.7 | 375 [M + H]⁺ | 1.80 (A) |
| 31.4 | 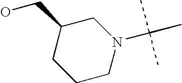 | III.7 | 433 [M + H]⁺ | 1.75 (A) |
| 31.5 | 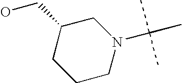 | III.7 | 433 [M + H]⁺ | 1.86 (A) |
| 31.6 | 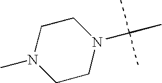 | III.6 | 418 [M + H]⁺ | 2.22 (A) |
| 31.7 | 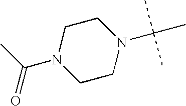 | III.6 | 446 [M + H]⁺ | 2.18 (A) |
| 31.8 | 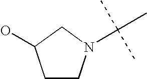 | III.6 | 405 [M + H]⁺ | 1.95 (A) |
| 31.9 | 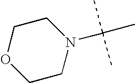 | III.6 | 405 [M + H]⁺ | 1.95 (A) |
| 31.10 | 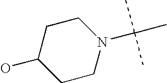 | III.6 | 419 [M + H]⁺ | 1.85 (A) |
| 31.11 | 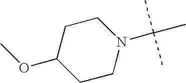 | III.6 | 433 [M + H]⁺ | 1.95 (A) |

The following compounds of general formula X can be prepared analogously to Example 30.1 or 30.2

| | |
|---|---|
| | (X) |
| 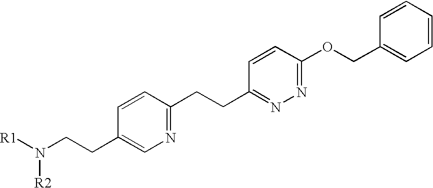 | |

| Example | R¹R²N— |
|---|---|
| 31.12 | 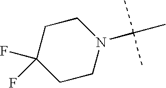 |
| 31.13 | 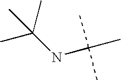 |
| 3114 | 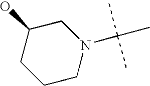 |
| 31.15 | 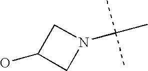 |
| 31.16 | 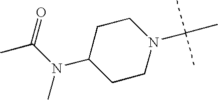 |
| 31.17 | 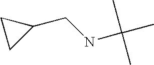 |
| 31.18 | 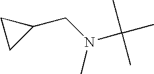 |
| 31.19 | 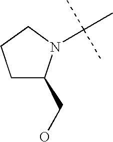 |
| 31.20 | 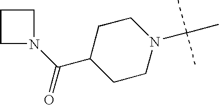 |
| 31.21 | 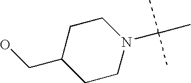 |
| 31.22 | 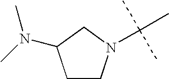 |
| 31.23 | 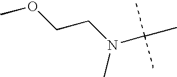 |
| 31.24 | 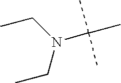 |
| 31.25 | 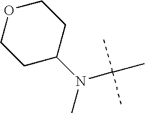 |
| 31.26 | 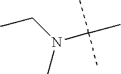 |
| 31.27 | 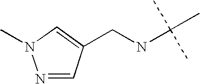 |
| 31.28 | 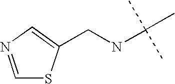 |
| 31.29 | 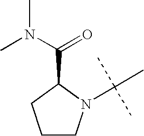 |
| 31.30 | 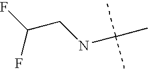 |
| 31.31 | 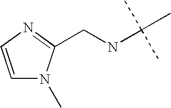 |
| 31.32 | 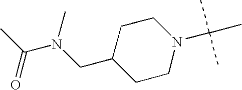 |
| 31.33 | 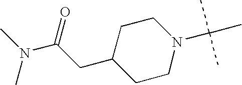 |
| 31.34 | 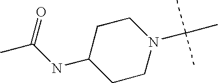 |
| 31.35 | 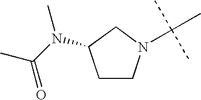 |
| 31.36 | 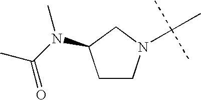 |
| 31.37 | 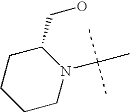 |
| 31.38 | 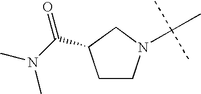 |
| 31.39 | 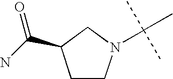 |
| 31.40 | 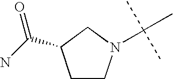 |
| 31.41 | 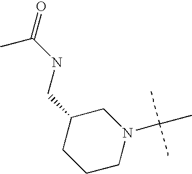 |
| 31.42 | 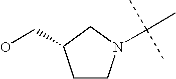 |
| 31.43 | 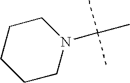 |

### Example 32.1

### 3-{4-[2-(6-Benzyloxy-pyridazin-3-yl)-ethyl]-benzyl}-pyrrolidine

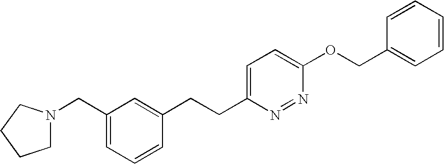

A mixture of 318 mg (1.0 mmol) 3-(6-benzyloxy-pyridazin-3-ylethyl)-benzaldehyde 0.093 ml (1.1 mmol) pyrrolidine, 2.42 g (5.0 mmol) MP-triacetoxy-borohydride (2.07 mmol/g, Argonaut) and 0.131 ml (2.3 mmol) of glacialic acid in 16 ml THF is shaken for 16 hours. The mixture is filtered and the filtrate is concentrated. The residue is purified by silica gel column chromatography with methylene chloride/MeOH/0.1% ammonia as eluent.

Yield: 260 mg (63% of theory),

retention time (HPLC): 2.18 min (method A)

C₂₄H₂₇N₃O

EII mass spectrum: m/z=374 [M+H]⁺

The following compounds of general formula XI are prepared analogously to Example XI, the educts used being shown in the column headed “Educts”:

| | |
|---|---|
| | (XI) |
| 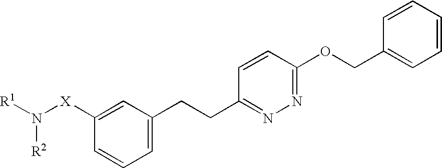 | |

| | | | | Retention |
|---|---|---|---|---|
| Exam- | | E- | mass | time |
| ple | R¹R²N—X— | ducts | spectrum | (HPLC) |
| 32.2 | 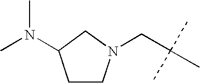 | I.14 | 417 [M + H]⁺ | 1.85 (A) |
| 32.3 | 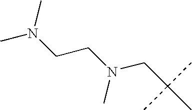 | I.14 | 405 [M + H]⁺ | 1.88 (A) |

### Example 33.1

### 3-Benzyloxy-6-{2-[3-(2-(4-methylpiperazin-1-yl)-ethyl)-phenyl]-ethyl}-pyridazine

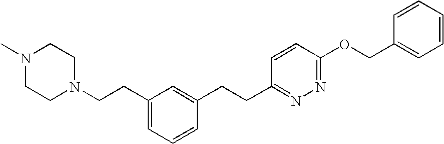

A mixture of 250 mg (0.61 mmol) of methanesulfonic acid 2-[3-(6-benzyloxy-pyridazin-3-ylethyl)-phenyl]-ethyl ester (example III.8), 335 mg (2.42 mmol) potassium carbonate and 0.067 ml (0.61 mmol) 4-methylpiperazine in 25 ml acetone is refluxed for 48 hours. The reaction mixture is concentrated. The residue is extracted with methylene chloride and water. The organic layer is dried over sodium sulphate and concentrated in vacuo. The residue is purified by silica gel column chromatography with methylene chloride/MeOH/0.1% ammonia as eluent. The solid is washed with diisopropylether and dried.

Yield: 174 mg (69% of theory),

retention time (HPLC): 2.12 min (method A)

C₂₆H₃₂N₄O

EII mass spectrum: m/z=417 [M+H]⁺

The following compounds of general formula XII are prepared analogously to Example 33.1, the educts used being shown in the column headed “Educts”:

| | |
|---|---|
| | (XII) |
| 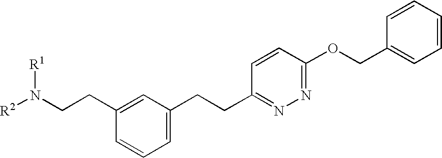 | |

| | | | | Retention |
|---|---|---|---|---|
| Exam- | | | mass | time |
| ple | R¹R²N— | Educts | spectrum | (HPLC) |
| 33.2 | 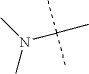 | III.8 | 362 [M + H]⁺ | 2.16 (A) |
| 33.3 | 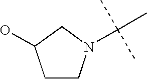 | III.8 | 404 [M + H]⁺ | 2.20 (A) |
| 33.4 | 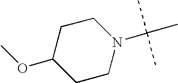 | III.9 | 432 [M + H]⁺ | 2.22 (A) |
| 33.5 | 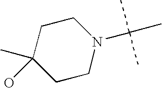 | III.8 | 432 [M + H]⁺ | 2.20 (A) |

### Example 34.1

### 3-Benzyloxy-6-{2-[3-(2-pyrrolidin-1-yl-ethoxy)-phenyl]-ethyl}-pyridazine

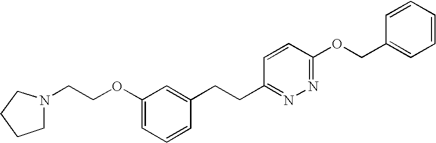

Prepared analogously to example 7.1 from Methanesulfonic acid 2-{3-[2-(6-benzyloxy-pyridazin-3-yl)-ethyl]-phenoxy}-ethyl ester and pyrrolidine.

Yield: 102 mg (36% of theory),

retention time (HPLC): 2.32 min (method A)

C₂₅H₂₉N₃O₂

EII mass spectrum: m/z=404 [M+H]⁺

The following compounds of general formula XIII are prepared analogously to Example 7.1, the educts used being shown in the column headed “Educts”:

| | |
|---|---|
| | (XIII) |
| 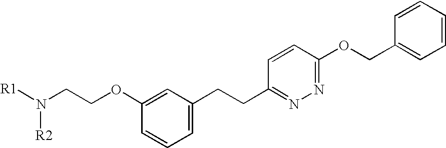 | |

| Exam- | | | mass | Retention time |
|---|---|---|---|---|
| ple | R¹R²N— | Educts | spectrum | (HPLC) |
| 34.2 | 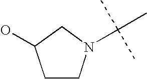 | IV.5 | 420 [M + H]⁺ | 2.25 (A) |
| 34.3 | 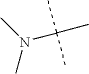 | IV.5 | 378 [M + H]⁺ | 2.29 (A) |
| 34.4 | 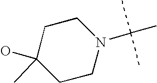 | IV.5 | 448 [M + H]⁺ | 2.23 (A) |
| 34.5 | 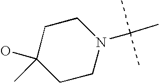 | IV.5 | 448 [M + H]⁺ | 2.34 (A) |

### Example 35.1

### 1-(2-{4-[2-(6-Benzyloxy-pyridazin-3-yl)-ethyl]-phenyl}-ethyl)-4-methyl-piperidin-4-ol

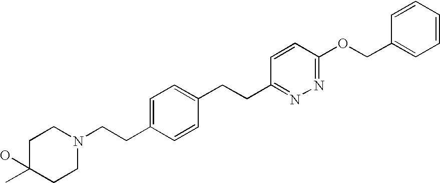

A mixture of 0.2 g (0.48 mmol) Methanesulfonic acid 2-{4-[2-(6-benzyloxy-pyridazin-3-yl)-ethyl]-phenyl}-ethyl ester, 58 mg (0.5 mmol) 4-Methyl-piperidin-4-ol, 75 mg (0.5 mmol) sodium iodide and 138 mg (1 mmol) potassium carbonate in 15 ml acetonitrile are refluxed for 48 hours. The mixture is evaporated and methylene chloride and water are added. The mixture is extracted. The organic phase is separated. Purification is achieved by HPLC (method 2).

Yield: 78 mg (37% of theory),

retention time (HPLC): 2.20 min (method A)

C₂₇H₃₃N₃O₂

EII mass spectrum: m/z=432 [M+H]⁺

The following compounds of general formula XIV are prepared analogously to Example 35.1, the educts used being shown in the column headed “Educts”:

| | |
|---|---|
| | (XIV) |
| 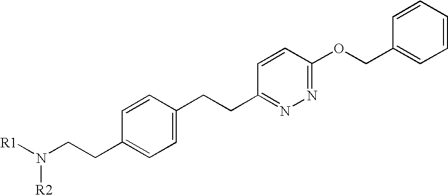 | |

| | | | | Retention |
|---|---|---|---|---|
| Exam- | | E- | mass | time |
| ple | R¹R²N— | ducts | spectrum | (HPLC) |
| 35.2 | 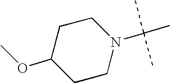 | III.10 | 432 [M + H]⁺ | 2.36 (A) |
| 35.3 | 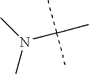 | III.10 | 404 [M + H]⁺ | 2.30 (A) |
| 35.4 | 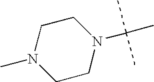 | III.9 | 417 [M + H]⁺ | 2.17 (A) |
| 35.5 | 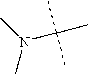 | III.9 | 362 [M + H]⁺ | 2.23 (A) |

The following compounds of general formula XIV can be prepared analogously to Example 35.1.

| | |
|---|---|
| | (XIV) |
| 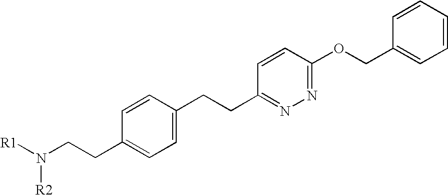 | |

| Example | R¹R²N— |
|---|---|
| 35.6 | 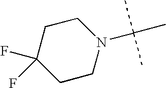 |
| 35.7 | 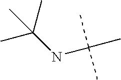 |
| 35.8 | 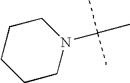 |
| 35.9 | 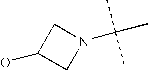 |
| 35.10 | 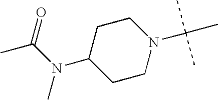 |
| 35.11 | 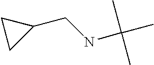 |
| 35.12 | 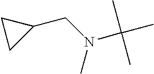 |
| 35.13 | 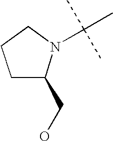 |
| 35.14 | 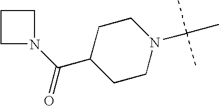 |
| 35.15 | 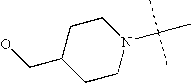 |
| 35.16 | 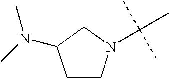 |
| 35.17 | 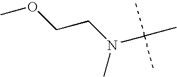 |
| 35.18 | 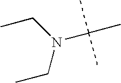 |
| 35.19 | 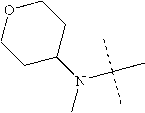 |
| 35.20 | 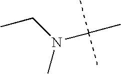 |
| 35.21 | 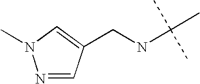 |
| 35.22 | 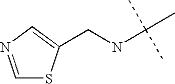 |
| 35.23 | 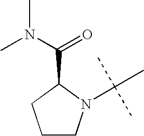 |
| 35.24 | 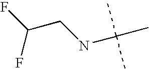 |
| 35.25 | 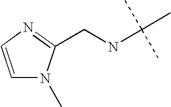 |
| 35.26 | 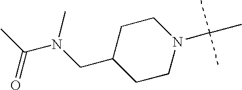 |
| 35.27 | 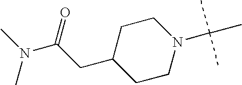 |
| 35.28 | 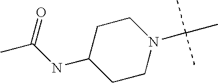 |
| 35.29 | 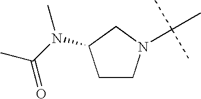 |
| 35.40 | 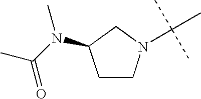 |
| 35.41 | 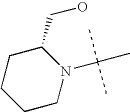 |
| 35.42 | 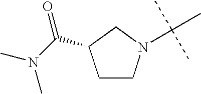 |
| 35.43 | 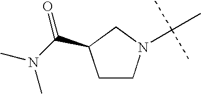 |
| 35.44 | 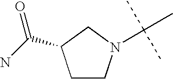 |
| 35.45 | 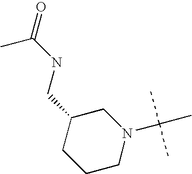 |
| 35.46 | 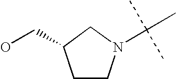 |
| 35.47 | 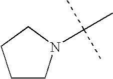 |

### Example 36.1

### 3-[2-(6-Benzyloxy-pyridazin-3-yl)-ethyl]-5,6,7,8-tetrahydro-[1,6]naphthyridine

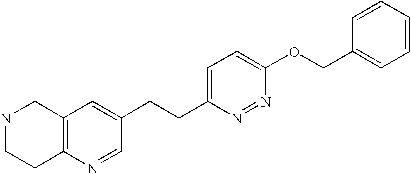

solution of 0.32 g (0.71 mmol) 3-[2-(6-Benzyloxy-pyridazin-3-yl)-ethyl]-7,8-dihydro-5H-[1,6]naphthyridine-6-carboxylic acid tert-butyl ester in 10 ml methylene chloride is treated with 0.55 ml trifluoroacetic acid and stirred at room temperature for 18 hours. 10 ml 2N sodium hydroxide solution is added and the layers are separated. The aqueous layer is extracted twice with methylene chloride and the combined organic layers are dried over sodium sulphate and concentrated. Purification is achieved by chromatography (silica gel, methylene chloride/methanol=6:4)

Yield: 110 mg (44% of theory),

C₂₁H₂₂N₄O

EII mass spectrum: m/z=347 [M+H]⁺

### Example 36.2

### 3-[2-(6-Benzyloxy-pyridazin-3-yl)-ethyl]-6-methyl-5,6,7,8-tetrahydro-[1,6]naphthyridine

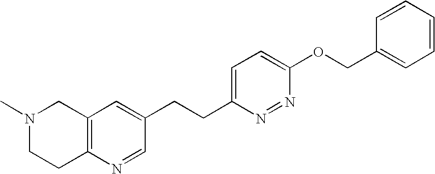

A solution of 80 mg (0.23 mmol) 3-[2-(6-Benzyloxy-pyridazin-3-yl)-ethyl]-5,6,7,8-tetrahydro-[1,6]naphthyridine in 6 ml THF is treated with 25 μl (0.34 mmol) 37% formaldehyde solution, 2 ml acetic acid and 73 mg (0.34 mmol) sodium triacetoxyborohydride and stirred for two hours at room temperature. THF is removed in vacuo and the residue is taken up in DMF. The product is purified by preparative HPLC (method 2). The collected product fractions are basified with 1N sodium hydroxide solution and extrated with methylene chloride. The organic layer is dried over sodium sulphate and concentrated in vacuo.

Yield: 32 mg (38% of theory),

C₂₂H₂₄N₄O

EII mass spectrum: m/z=361 [M+H]⁺

Some test methods for determining an MCH-receptor antagonistic activity will now be described. In addition, other test methods known to the skilled man may be used, e.g. by inhibiting the MCH-receptor-mediated inhibition of cAMP production, as described by Hoogduijn M et al. in “Melanin-concentrating hormone and its receptor are expressed and functional in human skin”, Biochem. Biophys. Res Commun. 296 (2002) 698-701 and by biosensory measurement of the binding of MCH to the MCH receptor in the presence of antagonistic substances by plasmon resonance, as described by Karlsson O P and Lofas S. in “Flow-Mediated On-Surface Reconstitution of G-Protein Coupled Receptors for Applications in Surface Plasmon Resonance Biosensors”, Anal. Biochem. 300 (2002), 132-138. Other methods of testing antagonistic activity to MCH receptors are contained in the references and patent documents mentioned hereinbefore, and the description of the test methods used is hereby incorporated in this application.

### MCH-1 Receptor Binding Test

Method: MCH binding to hMCH-1R transfected cells

### Species: Human

Test cell: hMCH-1R stably transfected into CHO/Galpha16 cells
Results: IC50 values

Membranes from CHO/Galpha16 cells stably transfected with human hMCH-1R are resuspended using a syringe (needle 0.6×25 mm) and diluted in test buffer (50 mM HEPES, 10 mM MgCl₂, 2 mM EGTA, pH 7.00; 0.1% bovine serum albumin (protease-free), 0.021% bacitracin, 1 μg/ml aprotinin, 1 μg/ml leupeptin and 1 μM phosphoramidone) to a concentration of 5 to 15 μg/ml.

200 microlitres of this membrane fraction (contains 1 to 3 μg of protein) are incubated for 60 minutes at ambient temperature with 100 pM of ¹²⁵I-tyrosyl melanin concentrating hormone (¹²⁵I-MCH commercially obtainable from NEN) and increasing concentrations of the test compound in a final volume of 250 microlitres. After the incubation the reaction is filtered using a cell harvester through 0.5% PEI treated fibreglass filters (GF/B, Unifilter Packard). The membrane-bound radioactivity retained on the filter is then determined after the addition of scintillator substance (Packard Microscint 20) in a measuring device (TopCount of Packard).

The non-specific binding is defined as bound radioactivity in the presence of 1 micromolar MCH during the incubation period.

The analysis of the concentration binding curve is carried out on the assumption of one receptor binding site.

### Standard:

Non-labelled MCH competes with labelled ¹²⁵I-MCH for the receptor binding with an IC50 value of between 0.06 and 0.15 nM.

The KD value of the radioligand is 0.156 nM.

### MCH-1 Receptor-Coupled Ca²⁺ Mobilisation Test

Method: Calcium mobilisation test with human MCH (FLIPR³⁸⁴)

### Species: Human

Test cells: CHO/Galpha 16 cells stably transfected with hMCH-R1
Results: 1st measurement: % stimulation of the reference (MCH 10⁻⁶M)
2nd measurement: pKB value

| | | |
|---|---|---|
| Reagents: | HBSS (10x) | (GIBCO) |
| | HEPES buffer (1M) | (GIBCO) |
| | Pluronic F-127 | (Molecular Probes) |
| | Fluo-4 | (Molecular Probes) |
| | Probenecid | (Sigma) |
| | MCH | (Bachem) |
| | bovine serum albumin | (Serva) |
| | (protease-free) | |
| | DMSO | (Serva) |
| | Ham's F12 | (BioWhittaker) |
| | FCS | (BioWhittaker) |
| | L-Glutamine | (GIBCO) |
| | Hygromycin B | (GIBCO) |
| | PENStrep | (BioWhittaker) |
| | Zeocin | (Invitrogen) |

Clonal CHO/Galpha16 hMCH-R1 cells are cultivated in Ham's F12 cell culture medium (with L-glutamine; BioWhittaker; Cat. No.: BE12-615F). This contains per 500 ml 10% FCS, 1% PENStrep, 5 ml L-glutamine (200 mM stock solution), 3 ml hygromycin B (50 mg/ml in PBS) and 1.25 ml zeocin (100 μg/ml stock solution). One day before the experiment the cells are plated on a 384-well microtitre plate (black-walled with a transparent base, made by Costar) in a density of 2500 cells per cavity and cultivated in the above medium overnight at 37° C., 5% CO₂ and 95% relative humidity. On the day of the experiment the cells are incubated with cell culture medium to which 2 mM Fluo-4 and 4.6 mM Probenicid have been added, at 37° C. for 45 minutes. After charging with fluorescent dye the cells are washed four times with Hanks buffer solution (1×HBSS, 20 mM HEPES), which has been combined with 0.07% Probenicid. The test substances are diluted in Hanks buffer solution, combined with 2.5% DMSO. The background fluorescence of non-stimulated cells is measured in the presence of substance in the 384-well microtitre plate five minutes after the last washing step in the FLIPR³⁸⁴ apparatus (Molecular Devices; excitation wavelength: 488 nm; emission wavelength: bandpass 510 to 570 nm). To stimulate the cells MCH is diluted in Hanks buffer with 0.1% BSA, pipetted into the 384-well cell culture plate 35 minutes after the last washing step and the MCH-stimulated fluorescence is then measured in the FLIPR³⁸⁴ apparatus.

### Data Analysis:

1st measurement: The cellular Ca²⁺ mobilisation is measured as the peak of the relative fluorescence minus the background and is expressed as the percentage of the maximum signal of the reference (MCH 10⁻⁶M). This measurement serves to identify any possible agonistic effect of a test substance.
2nd measurement: The cellular Ca²⁺ mobilisation is measured as the peak of the relative fluorescence minus the background and is expressed as the percentage of the maximum signal of the reference (MCH 10⁻⁶M, signal is standardised to 100%). The EC50 values of the MCH dosage activity curve with and without test substance (defined concentration) are determined graphically by the GraphPad Prism 2.01 curve program. MCH antagonists cause the MCH stimulation curve to shift to the right in the graph plotted.

The inhibition is expressed as a pKB value:

pKB=log(EC_{50(testsubstance+MCH)}/EC_{50(MCH)}−1)−log c_{(testsubstance)}

The compounds according to the invention, including their salts, exhibit an MCH-receptor antagonistic activity in the tests mentioned above. Using the MCH-1 receptor binding test described above an antagonistic activity is obtained in a dosage range from about 10⁻¹⁰ to 10⁻⁵ M, particularly from 10⁻⁹ to 10⁻⁶ M.

In order to illustrate that compounds according to the invention with different structural elements possess a good to very good MCH-1 receptor antagonistic activity, the IC₅₀ values of the compounds depicted in the following table are provided. It is noted that the compounds are selected in view of their different structural elements by way of example without any intent to highlight any specific compound.

| Compound | |
|---|---|
| according to | IC₅₀ value |
| Example no. | [nM] |
| 1.1 | 4 |
| 4.1 | 16 |
| 4.2 | 20 |
| 4.4 | 8 |
| 8.10 | 27 |
| 10.5 | 4 |
| 12.2 | 13 |
| 15.1 | 9 |
| 16.2 | 590 |
| 22.2 | 136 |
| 29.10 | 19 |
| 31.10 | 4 |

Some examples of formulations will now be described in which the term “active substance” denotes one or more compounds according to the invention, including the salts thereof. In the case of one of the combinations with one or additional active substances as described previously, the term “active substance” also includes the additional active substances.

### Example A

Tablets containing 100 mg of active substance

### Composition:

1 tablet contains:

| | | |
|---|---|---|
| active substance | 100.0 | mg |
| lactose | 80.0 | mg |
| corn starch | 34.0 | mg |
| polyvinylpyrrolidone | 4.0 | mg |
| magnesium stearate | 2.0 | mg |
| | 220.0 | mg |

### Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.

Weight of tablet: 220 mg

Diameter: 10 mm, biplanar

### Example B

Tablets containing 150 mg of active substance

### Composition:

1 tablet contains:

| | | |
|---|---|---|
| active substance | 150.0 | mg |
| powdered lactose | 89.0 | mg |
| corn starch | 40.0 | mg |
| colloidal silica | 10.0 | mg |
| polyvinylpyrrolidone | 10.0 | mg |
| magnesium stearate | 1.0 | mg |
| | 300.0 | mg |

### Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.

Weight of tablet: 300 mg

die: 10 mm, flat

### Example C

Hard gelatine capsules containing 150 mg of active substance

### Composition:

1 capsule contains:

| | | |
|---|---|---|
| active substance | 150.0 | mg |
| corn starch (dried) | approx. 180.0 | mg |
| lactose (powdered) | approx. 87.0 | mg |
| magnesium stearate | 3.0 | mg |
| | approx. 420.0 | mg |

### Preparation:

The active substance is mixed with the excipients, passed through a screen with a mesh size of 0.75 mm and homogeneously mixed using a suitable apparatus. The finished mixture is packed into size 1 hard gelatine capsules.

Capsule filling: approx. 320 mg

Capsule shell: size 1 hard gelatine capsule.

### Example D

Suppositories containing 150 mg of active substance

### Composition:

1 suppository contains:

| | |
|---|---|
| active substance | 150.0 mg |
| polyethyleneglycol 1500 | 550.0 mg |
| polyethyleneglycol 6000 | 460.0 mg |
| polyoxyethylene sorbitan monostearate | 840.0 mg |
| | 2,000.0 mg |

### Preparation:

After the suppository mass has been melted the active substance is homogeneously distributed therein and the melt is poured into chilled moulds.

### Example E

Ampoules containing 10 mg active substance

### Composition:

| active substance | 10.0 | mg |
|---|---|---|
| 0.01 N hydrochloric acid | q.s. | |
| double-distilled water | ad 2.0 | ml |

### Preparation:

The active substance is dissolved in the necessary amount of 0.01 N HCl, made isotonic with common salt, filtered sterile and transferred into 2 ml ampoules.

### Example F

Ampoules containing 50 mg of active substance

### Composition:

| active substance | 50.0 | mg |
|---|---|---|
| 0.01 N hydrochloric acid | q.s. | |
| double-distilled water | ad 10.0 | ml |

### Preparation:

The active substance is dissolved in the necessary amount of 0.01 N HCl, made isotonic with common salt, filtered sterile and transferred into 10 ml ampoules.

1. Compounds of general formula I 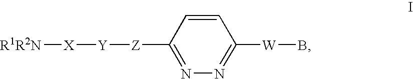
wherein:
R¹ and R² are independently H, C₁₋₈-alkyl, or C₃₋₇-cycloalkyl, wherein the alkyl or cycloalkyl group thereof is optionally independently mono- or polysubstituted by R¹¹, and a —CH₂— group in position 3 or 4 of a 5-, 6-, or 7-membered cycloalkyl group is optionally replaced by —O—, —S—, or —NR¹³—; or
R² is a C₁₋₃-alkylene bridge which is linked to Y, wherein the alkylene bridge optionally substituted with one or more C₁₋₃-alkyl-groups, and R¹ is defined as hereinbefore or is a group selected from C₁₋₄-alkyl-CO—, C₁₋₄-alkyl-O—CO—, (C₁₋₄-alkyl)NH—CO—, or (C₁₋₄-alkyl)₂N—CO—, wherein alkyl-groups are optionally mono- or polyfluorinated; or
R¹ and R² form a C₃₋₈-alkylene bridge, wherein a —CH₂— group not adjacent to the N atom of the R¹R²N— group is optionally replaced by —CH═N—, —CH═CH—, —O—, —S—, —SO—, —(SO₂)—, —CO—, —C(═CH₂)—, —C(═N—O—(C₁₋₄-alkyl))-, or —NR¹³—,
wherein if R¹ and R² form an alkylene bridge, in the alkylene bridge one or more H atoms are optionally replaced by identical or different groups R¹⁴, and the alkylene bridge is optionally substituted by one or two identical or different carbo- or heterocyclic groups Cy in such a way that the bond between the alkylene bridge and the group Cy is made
via a single or double bond,
via a common C atom forming a spirocyclic ring system,
via two common adjacent C and/or N atoms forming a fused bicyclic ring system, or
via three or more C and/or N atoms forming a bridged ring system;
X is a bridging group selected from the group consisting of —CH₂—, —CH₂—CH₂—, —CH₂—CH₂—CH₂—, —CH₂—CH₂—O—, and —CH₂—CH₂—NR^{N}, each optionally comprising one, two, or three identical or different C₁₋₄-alkyl substituents, wherein two alkyl groups are optionally joined together forming a 3- to 7-membered cyclic group, and wherein in a C₂₋₃-alkylene bridge one or two C atoms are optionally monosubstituted by R¹⁰; and
R¹⁰ is hydroxy, hydroxy-C₁₋₃-alkyl, C₁₋₄-alkoxy, or C₁₋₄-alkoxy-C₁₋₃-alkyl; and
Y is a 5- to 6-membered aromatic carbocyclic group, optionally containing 1, 2, or 3 heteroatoms independently selected from N, O, and/or S, which cyclic group is optionally mono- or polysubstituted by identical or different substituents R²⁰;
Z is —CH₂—CH₂—, —C(═O)—CH₂—, —C(═CH₂)—CH₂—, —C(OH)H—CH₂—, or —CH₂—C(OH)H—, all of which are optionally independently mono- or polysubstituted with substituents selected from C₁₋₃-alkyl;
W is —CH₂—CH₂—, —CH₂—O—, —O—CH₂—, —CH═CH—, —CH₂—NR^{N}—, —NR^{N}—CH₂—, —CH₂—, —O—, —S—, or —NR^{N}—, wherein one or more H atoms are optionally replaced independently of each other by C₁₋₃-alkyl;
R^{N} are independently H, C₁₋₄-alkyl, formyl, C₁₋₃-alkylcarbonyl, or C₁₋₃-alkylsulfonyl; and
B is a 5- or 6-membered unsaturated or aromatic carbocyclic group containing 1, 2, 3, or 4 heteroatoms independently selected from N, O, and/or S; which cyclic group is optionally mono- or polysubstituted by identical or different substituents R²⁰, or
is C₁₋₆-alkyl, C₃₋₇-cycloalkyl, or C₃₋₇-cycloalkyl-C₁₋₃-alkyl, each optionally independently mono- or poly-substituted by R¹⁴, and wherein the cycloalkyl-groups one or two —CH₂-groups are optionally independently replaced by —O—, —S—, —NR¹³—, or —C(═O)—; and
Cy is a carbo- or heterocyclic group selected from:
a saturated 3- to 7-membered carbocyclic group,
an unsaturated 4- to 7-membered carbocyclic group,
a phenyl group,
a saturated 4- to 7-membered or unsaturated 5- to 7-membered heterocyclic group with an N, O, or S atom as heteroatom,
a saturated or unsaturated 5- to 7-membered heterocyclic group with two or more N atoms or with one or two N atoms and an O or S atom as heteroatoms,
an aromatic heterocyclic 5- or 6-membered group with one or more identical or different heteroatoms selected from N, O, and/or S,
wherein the above-mentioned saturated 6- or 7-membered groups may also be present as bridged ring systems with an imino, (C₁₋₄-alkyl)-imino, methylene, ethylene, (C₁₋₄-alkyl)-methylene, or di-(C₁₋₄-alkyl)-methylene bridge, and
wherein the above-mentioned cyclic groups are optionally mono- or polysubstituted at one or more C atoms by identical or different groups R²⁰, or in the case of a phenyl group are optionally additionally monosubstituted by nitro, and/or one or more NH groups are optionally substituted by R²¹; and
wherein in the above-mentioned saturated or unsaturated carbo- or heterocyclic groups a —CH₂-group is optionally replaced by a —C(═O)— group;
R¹¹ is halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N—, R¹⁸R¹⁹N—CO—, or Cy, while in the above-mentioned groups one or more C atoms are optionally independently substituted by halogen, OH, CN, CF₃, C₁₋₃-alkyl, C₁₋₃-alkoxy, or hydroxy-C₁₋₃-alkyl;
R¹³ is R¹⁷ or formyl;
R¹⁴ is halogen, cyano, C₁₋₆-alkyl, C₂₋₆-alkenyl, C₂₋₆-alkynyl, R¹⁵—O—, R¹⁵—O—CO—, R¹⁵—CO—, R¹⁵—CO—O—, R¹⁶R¹⁷N—, HCO—NR¹⁵—, R¹⁸R¹⁹N—CO—, R¹⁸R¹⁹N—CO—NH—, R¹⁵—O—C₁₋₃-alkyl, R¹⁵—O—CO—C₁₋₃-alkyl-, R¹⁵—SO₂—NH—, R¹⁵—SO₂—N(C₁₋₃-alkyl)-, R¹⁵—O—CO—NH—C₁₋₃-alkyl-, R¹⁵—SO₂—NH—C₁₋₃-alkyl-, R¹⁵—CO—C₁₋₃-alkyl-, R¹⁵—CO—O—C₁₋₃-alkyl-, R¹⁶R¹⁷N—C₁₋₃-alkyl-, R¹⁸R¹⁹N—CO—C₁₋₃-alkyl- or Cy-C₁₋₃-alkyl-,
R¹⁵ is H, C₁₋₄-alkyl, C₃₋₇-cycloalkyl, C₃₋₇-cycloalkyl-C₁₋₃-alkyl, phenyl, phenyl-C₁₋₃-alkyl, pyridinyl, or pyridinyl-C₁₋₃-alkyl,
R¹⁶ is H, C₁₋₆-alkyl, C₃₋₇-cycloalkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl, or cyclo-C₃₋₆-alkyleneimino-C₂₋₆-alkyl,
R¹⁷ is R¹⁶, phenyl, phenyl-C₁₋₃-alkyl, pyridinyl, C₁₋₄-alkylcarbonyl, C₃₋₇-cycloalkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N—(C₁₋₄-alkylcarbonyl)-N—(C₁₋₄-alkyl)-amino-C₂₋₃-alkyl, C₁₋₄-alkylamino-carbonyl, C₁₋₄-alkylsulphonyl, C₁₋₄-alkylsulphonylamino-C₂₋₃-alkyl, or N—(C₁₋₄-alkylsulphonyl)-N(—C₁₋₄-alkyl)-amino-C₂₋₃-alkyl;
R¹⁸ and R¹⁹ are independently H or C₁₋₆-alkyl wherein R¹⁸ and R¹⁹ are optionally linked to form a C₃₋₆-alkylene bridge, wherein a —CH₂— group not adjacent to an N atom is optionally replaced by —O—, —S—, —SO—, —(SO₂)—, —CO—, —C(═CH₂)—, or —NR¹³—;
R²⁰ is halogen, hydroxy, cyano, nitro, C₁₋₆-alkyl, C₂₋₆-alkenyl, C₂₋₆-alkynyl, C₃₋₇-cycloalkyl, hydroxy-C₁₋₃-alkyl, R²²—C₁₋₃-alkyl, or R²²; and
R²¹ is C₁₋₄-alkyl, ω-hydroxy-C₂₋₆-alkyl, ω-C₁₋₄-alkoxy-C₂₋₆-alkyl, ω-C₁₋₄-alkylamino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulphonyl, aminosulphonyl, C₁₋₄-alkylaminosulphonyl, di-C₁₋₄-alkylaminosulphonyl, or cyclo-C₃₋₆-alkylene-imino-sulphonyl,
R²² is pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, cyclo-C₃₋₆-alkyl-amino-carbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulphonyl, C₁₋₄-alkyl-sulphinyl, C₁₋₄-alkyl-sulphonylamino, C₁₋₄-alkyl-sulphonyl-N—(C₁₋₄-alkyl)amino, amino, C₁₋₄-alkylamino, di-(C₁₋₄-alkyl)-amino, C₁₋₄-alkyl-carbonyl-amino, C₁₋₄-alkyl-carbonyl-N—(C₁₋₄-alkyl)amino, cyclo-C₃₋₆-alkyleneimino, phenyl-C₁₋₃-alkylamino, N—(C₁₋₄-alkyl)-phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, aminocarbonylamino, or C₁₋₄-alkylaminocarbonylamino,
wherein in the above-mentioned groups and radicals one or more C atoms are optionally additionally mono- or polysubstituted by F and/or in each case one or two C atoms are optionally additionally independently monosubstituted by Cl or Br and/or in each case one or more phenyl rings optionally additionally independently comprise one, two, or three substituents selected from the group F, Cl, Br, I, cyano, C₁₋₄ alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl-, and di-(C₁₋₃-alkyl)-amino-C₁₋₃-alkyl, and/or are optionally monosubstituted by nitro, and the H atom of any carboxy group present or an H atom bound to an N atom is optionally replaced by a group which can be cleaved in vivo, or
a tautomers, the diastereomers, enantiomers, or salt thereof.

2. The Compound according to claim 1, wherein R¹ and R² are independently H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl C₁₋₃ alkyl, (hydroxy-C₃₋₇-cycloalkyl)-C₁₋₃-alkyl, hydroxy-C₂₋₄-alkyl, C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N—(C₁₋₄-alkyl)-pyrrolidin-3-yl, pyrrolidinyl-C₁₋₃-alkyl, N—(C₁₋₄-alkyl)-pyrrolidinyl-C₁₋₃-alkyl, piperidin-3-yl, piperidin-4-yl, N—(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-yl, tetrahydropyran-4-yl, tetrahydrofuran-2-ylmethyl, tetrahydrofuran-3-ylmethyl, phenyl-C₁₋₃-alkyl, or pyridyl-C₁₋₃-alkyl, wherein in the above-mentioned groups and radicals one or more C atoms are optionally independently mono- or polysubstituted by F, C₁₋₃-alkyl, or hydroxy-C₁₋₃-alkyl, and/or one or two C atoms are optionally independently monosubstituted by Cl, Br, OH, CF₃, or CN, and the above-mentioned cyclic groups are optionally mono- or polysubstituted at one or more C atoms by identical or different radicals R²⁰, in the case of a phenyl group are optionally additionally monosubstituted by nitro, and/or one or more NH groups are optionally substituted by R²¹.

3. The Compound according to claim 1, wherein R¹ and R² together with the N atom to which they are bound form a heterocyclic group selected from azetidine, pyrrolidine, piperidine, azepan, 2,5-dihydro-1H-pyrrole, 1,2,3,6-tetrahydro-pyridine, 2,3,4,7-tetrahydro-1H-azepine, 2,3,6,7-tetrahydro-1H-azepine, piperazine in which the free imine function is substituted by R¹³, piperidin-4-one, morpholine, thiomorpholine, 1-oxo-thiomorpholin-4-yl, and 1,1-dioxo-thiomorpholin-4-yl;
wherein one or more H atoms are optionally replaced by identical or different groups R¹⁴, and/or
the heterocyclic groups specified are optionally substituted by one or two identical or different carbo- or heterocyclic groups Cy in such a way that the bond between the alkylene bridge and the group Cy is made
via a single or double bond,
via a common C atom forming a spirocyclic ring system,
via two common adjacent C and/or N atoms forming a fused bicyclic ring system, or
via three or more C and/or N atoms forming a bridged ring system.

4. The Compound according to claim 1, wherein X is a —CH₂—, —CH₂—CH₂—, —CH₂—CH₂—CH₂—, —CH₂—CH₂—O—, or —CH₂—CH₂—NR^{N}—, wherein one or two hydrogen atoms are optionally replaced by identical or different C₁₋₃-alkyl-groups, wherein two alkyl-groups are optionally linked together to form a 3- to 6-membered cycloalkyl group.

5. The Compound according to claim 1, wherein Y is phenyl, pyridyl, pyridazinyl, pyrimidinyl, pyrazinyl, thiazolyl, furyl, or thiophenyl, each optionally mono- or polysubstituted by identical or different substituents R²⁰.

6. The Compound according to claim 1, wherein Z is a group selected from —CH₂—CH₂—, —C(═O)—CH₂—, —C(OH)H—CH₂—, —CH₂—C(═O)—, or —CH₂—C(OH)H—, wherein one or more H atoms are optionally replaced by F atoms.

7. The Compound according to claim 1, wherein W is —CH₂—CH₂—, —O—CH(CH₃)—, or —NR^{N}—CH₂—, wherein one or more H atoms are optionally replaced by F atoms.

8. The Compound according to claim 1, wherein B is phenyl, pyridyl, pyridazinyl, pyrazinyl, pyrimidinyl, pyrrolyl, pyrazolyl, imidazolyl, triazolyl, tetrazolyl, furyl, thiophenyl, or thiazolyl, each optionally mono- or polysubstituted by identical or different substituents R²⁰.

9. The Compound according to claim 1, wherein:
R²⁰ is halogen, hydroxy, cyano, nitro, C₁₋₄-alkyl, C₁₋₄-alkoxy, hydroxy-C₁₋₄-alkyl, (C₁₋₃-alkyl)-carbonyl-, di-(C₁₋₃-alkyl)amino, aminocarbonyl, (C₁₋₃-alkyl)-carbonylamino, (C₁₋₃-alkyl)-sulfonylamino or R²²—C₁₋₃-alkyl, wherein one or more C atoms are optionally additionally mono- or polysubstituted by F and/or in each case one or two C atoms are optionally independently additionally monosubstituted by Cl or Br; and
R²² is C₁₋₄-alkoxy, C₁₋₄-alkylcarbonyl, amino, C₁₋₄-alkylamino, or di-(C₁₋₄-alkyl)-amino, wherein one or more H atoms are optionally replaced by F atoms.

10. A physiologically acceptable salts of the compound according to one of claims 1 to 9.

11.-23. (canceled)
